(19) United States
(12) Patent Application Publication
(10) Pub. No.: US 2025/0144104 A1
(43) Pub. Date: May 8, 2025

(54) DISCOVERY OF A NEW INHIBITOR FOR THE YTH DOMAIN-CONTAINING M6A RNA READERS

(71) Applicant: The Trustees of Boston College, Chestnut Hill, MA (US)

(72) Inventors: Huiqing Zhou, Chestnut Hill, MA (US); Chuan-Hui Wang, Boston, MA (US)

(21) Appl. No.: 18/937,951

(22) Filed: Nov. 5, 2024

Related U.S. Application Data
(60) Provisional application No. 63/596,418, filed on Nov. 6, 2023.

(51) Int. Cl. A61K 31/52; A61K 31/5377; C12N 15/10

(52) U.S. Cl. CPC A61K 31/52; A61K 31/5377; C12N 15/1062

(57) ABSTRACT

The present invention discloses a method for inhibiting a pan-YTH domain by contacting the pan-YTH domain with a small molecule pan-YTH domain inhibitor. Novel methods of locating the small molecule inhibitors are discussed for high-throughput screening (HTS). The small molecule pan-YTH domain inhibitor is designed to inhibit multiple YTH domains present in a YTH family of proteins that identify m6A-modified transcripts, thereby altering the post-transcriptional modification process.

### CROSS-REFERENCE TO RELATED APPLICATIONS

This application claims the benefit of priority to U.S. Provisional Patent No. 63/596,418, filed on 6 Nov. 2023, the entirety of which is incorporated by reference as if fully reproduced and set forth herein in its entirety.

### STATEMENT REGARDING FEDERALLY SPONSORED RESEARCH OR DEVELOPMENT

This invention was made with government support under R35 GM150789 awarded by the National Institutes of Health. The government has certain rights in the invention.

### BRIEF DESCRIPTION OF THE SEQUENCE LISTING

Not applicable.

### FIELD OF THE INVENTION

This invention relates to the identification of new small-molecule inhibitors of pan-YTH domain.

### BACKGROUND OF THE INVENTION

In recent years, efforts to study YTH domains have involved utilizing RNA-based reagents, such as antisense oligonucleotides or small interfering RNAs, to target the expression of individual YTH domain-containing proteins. By specifically targeting the mRNA encoding a particular YTH domain, these RNA-based reagents aim to reduce the levels of the YTH domain protein and consequently inhibit its function in recognizing m6A-modified transcripts. However, the specificity and efficiency of targeting multiple YTH domains within the YTH family without reducing protein production levels remain a challenge, as unintended consequences may arise from the reduction of YTH domain protein level. What is urgently needed are new inhibitors for the YTH domain-containing m6A RNA readers.

### BRIEF SUMMARY OF THE INVENTION

The following presents a simplified summary of the innovation in order to provide a basic understanding of some aspects of the invention. This summary is not an extensive overview of the invention. It is intended to neither identify key or critical elements of the invention nor delineate the scope of the invention. Its sole purpose is to present some concepts of the invention in a simplified form as a prelude to the more detailed description that is presented later.

In recent years, the study of RNA modifications has gained significant attention due to their crucial role in regulating gene expression and maintaining cellular function. Among these modifications, N6-methyladenosine (m6A) is the most prevalent internal modification in eukaryotic messenger RNA (mRNA) and has been implicated in various biological processes, including mRNA stability, splicing, translation, and decay. The m6A modification is recognized by specific proteins, such as those in the YTH domain family, which bind to m6A-modified transcripts and mediate their downstream effects. These YTH domain-containing proteins are essential for the proper functioning of m6A-related pathways and have been linked to several diseases, including cancer, neurological disorders, and metabolic diseases.

Despite the critical role of YTH domain proteins in m6A recognition and the regulation of gene expression, there is a growing need for targeted approaches to modulate their activity. Traditional methods of inhibiting protein function often involve genetic manipulation or the use of large biomolecules, which can be challenging to deliver and laborious, lack specificity, limited for applications in vivo or achieving temporal controls of the perturbation. As a result, there is an increasing interest in developing small molecule inhibitors that can selectively target these proteins, offering a more precise and potentially therapeutic approach to modulating m6A-related pathways. Such advancements could lead to novel treatments for diseases where dysregulation of m6A modifications and YTH domain proteins play a pivotal role.

N⁶-methyladenosine (m⁶A) is an abundant modification in mammalian mRNAs and plays important regulatory functions in gene expression, primarily mediated through specific recognition by “reader” proteins. YTH family proteins are one major family of known m⁶A readers, which specifically recognize m⁶A-modified transcripts via the YTH domains. Despite the significant relevance of YTH-m⁶A recognition in biology and diseases, few small molecule inhibitors are available for specifically perturbing this interaction. Herein we elucidate and expand upon the discovery of a new inhibitor (“N-7”) for the YTH-m⁶A RNA recognition, from the screening of a nucleoside analogue library against the YTH domain of the YTHDF1 protein. N-7 is characterized to be apan-inhibitor in vitro against five YTH domains from human YTHDF1, YTHDF2, YTHDF3, YTHDC1, and YTHDC2 proteins, with IC₅₀ ranges within 30-48 μM measured by the fluorescence polarization competition assay. We demonstrated that N-7 directly interacts with the YTH domain proteins via thermal shift assay. N-7 expands the chemical structure landscape of the m⁶A YTH domain reader inhibitors and potentiates future inhibitor development for reader functional studies and therapeutic efforts in targeting the epitranscriptome.

In some embodiments, the technology disclosed herein provides a method for inhibiting the pan-YTH domain by a small molecule comprising Formula 1:

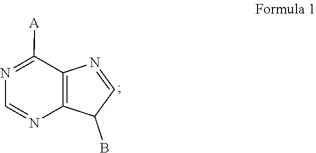

and/or a pharmaceutically acceptable salt thereof, hydrate and/or solvate thereof, tautomer thereof, and/or a prodrug thereof of Formula 1.

The technology contemplates that A and B can be any substituent suitable to carry out the method.

The approach herein features screening against one YTH domain, rather than multiple. The inhibitor was confirmed post-screening to be a pan inhibitor.

For example, in some embodiments, A and B shown above are independently selected from O, S, CH₂, NR¹¹, C═CH₂, SO, SO₂, NR¹⁻¹²; and/or R¹⁻¹². R¹ is selected from H, S—R¹³, SO—R¹³, and SO₂R¹³; R² and R³ independently are selected from H, C₁₋₄ alkyl, C₃₋₅-cycloalkyl, C₂₋₄ alkene; C₃₋₅-cycloalkene, alternatively, R² and R³ along with the carbon atom to which they are attached represent C═O, C═S, or C═CH₂; R⁴ is selected from H, C₁₋₄ alkyl, C₃₋₅-cycloalkyl, C₂₋₄ alkene, C₃₋₅-cycloalkene, SO—C₁₋₄ alkyl, and SO₂—C₁₋₄ alkyl; R⁵ is selected from H, C₁₋₄ alkyl, C₁₋₄ substituted alkyl, C₃₋₁₀-cycloalkyl, bicycloalkyl, C₃₋₁₀-heterocycloalkyl, aryl, substituted aryl, aryl-S(O)ₙ—C₁₋₄ alkyl, heteroaryl, (CH₂)₀₋₅-heterocycloalkyl, and (CH₂)₁₋₅—N(C₁₋₄ alkyl)₂; alternatively, A and/or B along with the carbon atom or nitrogen atom to which they are attached represent a substituted or unsubstituted saturated or unsaturated hetero-cycloalkyl ring; R¹¹ and R¹² independently are selected from H, C₁₋₄ alkyl, C₃₋₅-cycloalkyl, C₂₋₄ alkene, C₃₋₅-cycloalkene, SO—C₁₋₄ alkyl, and SO₂—C₁₋₄ alkyl; and R¹³ is selected from C₁₋₄ alkyl, C₃₋₅-cycloalkyl, C₂₋₄ alkene, and C₃₋₅-cycloalkene; and n represents an integer from 0 to 2.

According to some aspects, the method above is wherein: A and B are each independently selected from H, SO—R¹³, and SO₂R¹³; or A and B independently are selected from H, C₁₋₄ alkyl, and C₃₋₅-cycloalkyl; alternatively, R² and R³ along with the carbon atom to which they are attached represent C═O or C═CH₂; R⁴ is selected from H, C₁₋₄ alkyl, C₃₋₅-cycloalkyl, and SO₂—C₁₋₄ alkyl; R⁵ is selected from H, C₁₋₂ alkyl, C₁₋₄ substituted alkyl, C₅₋₉-cycloalkyl, C₆₋₁₀ aryl, C₆₋₁₀ substituted aryl, aryl-S(O)ₙ—C₁₋₄ alkyl, C₅₋₆-heteroaryl, (CH₂)₀₋₅-heterocycloalkyl, bicycloheptane, (CH₂)₁₋₄—N(C₁₋₃ alkyl)₂ and C₄₋₆ heterocycloalkyl; alternatively, R⁴ and R⁵ along with the nitrogen atom to which they are attached represent a substituted or unsubstituted saturated or unsaturated C₄₋₁₀ hetero-cycloalkyl ring; R¹³ is selected from C₁₋₂ alkyl, and C₃₋₄-cycloalkyl; and n represents an integer from 0 to 1.

In some embodiments, the technology provides a method for inhibiting a pan-YTH domain inhibitor by contacting the inhibitor with is a small molecule comprising Formula 1:

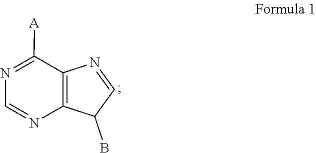

wherein each independent occurrence of A and/or B is independently selected from and/or comprises one or more of the following substituents and/or a salt, tautomer, hydrate and/or solvate thereof:

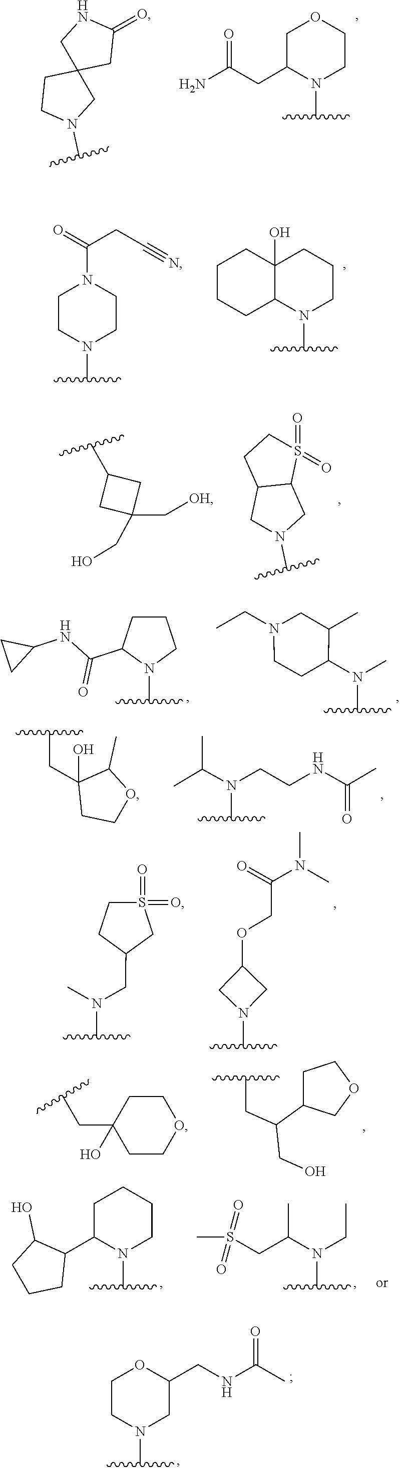

wherein each independent occurrence of [symbol] is a linker to Formula 1 and represents an attachment to an atom as indicated in Formula 1 and included or is a combination of a single bond (−), a bond to a nitrogen, a bond to a carbon, a bond to an oxygen (—O—), a double bond (=), a triple bond (≡) or (—C₂H₂—), a bond to C—C, a bond to C—N, —(CH₂)ₙ—, wherein independently each n=0, 1, 2, 3, 4, 5, 6, 7, 8, or 9; or a combination thereof.

According to some aspects, the method can inhibit the interaction between YTH domains and m⁶A-modified transcripts. In some embodiments, a YTH domain is part of the YTH family of proteins.

In some embodiments, the small molecule comprises:

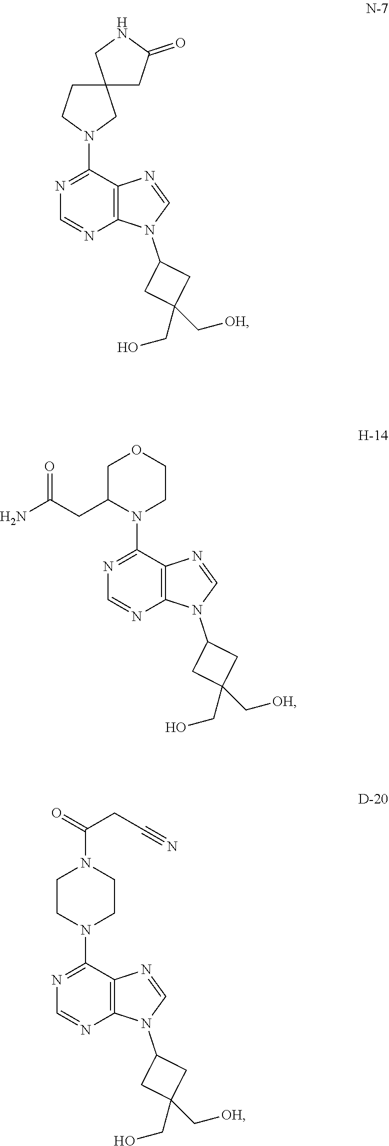 [SMILES: O=C1CC2(CCN(c3ncnc4c3ncn4C3CC(CO)(CO)C3)C2)CN1 ; NC(=O)CC1COCCN1c1ncnc2c1ncn2C1CC(CO)(CO)C1 ; N#CCC(=O)N1CCN(c2ncnc3c2ncn3C2CC(CO)(CO)C2)CC1]

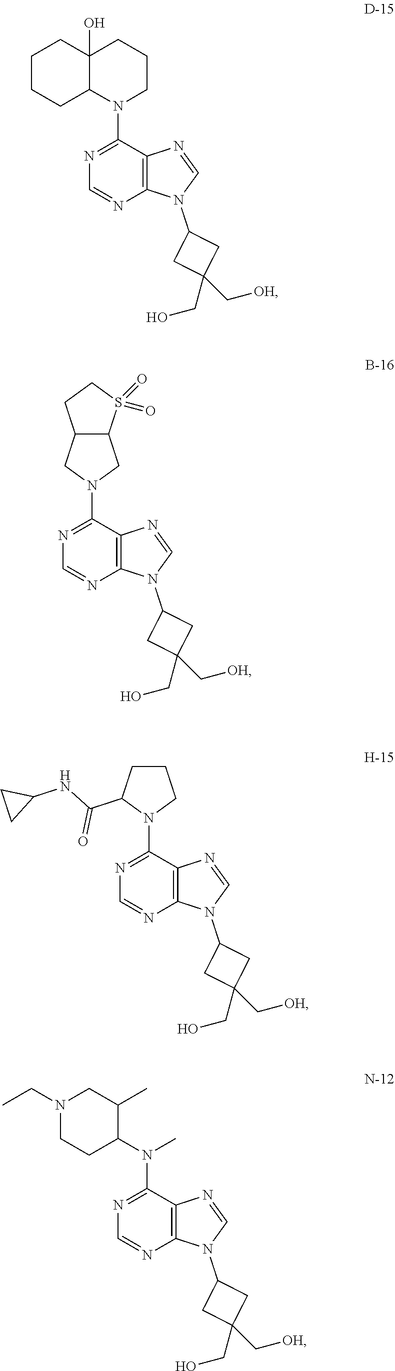 [SMILES: OCC1(CO)CC(n2cnc3c(N4CCCC5(O)CCCCC45)ncnc32)C1 ; O=S1(=O)CCC2CN(c3ncnc4c3ncn4C3CC(CO)(CO)C3)CC21 ; O=C(NC1CC1)C1CCCN1c1ncnc2c1ncn2C1CC(CO)(CO)C1 ; CCN1CCC(N(C)c2ncnc3c2ncn3C2CC(CO)(CO)C2)C(C)C1]

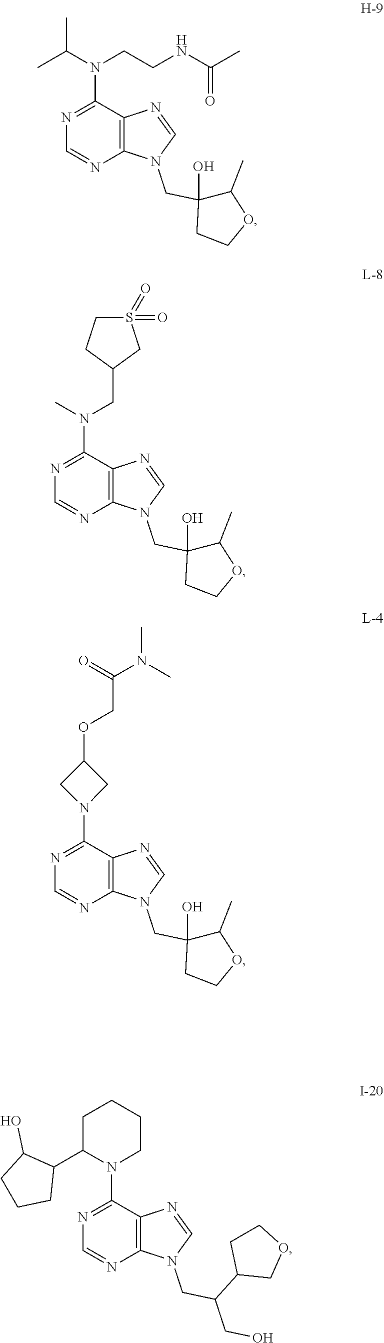 [SMILES: CC(=O)NCCN(c1ncnc2c1ncn2CC1(O)CCOC1C)C(C)C ; CC1OCCC1(O)Cn1cnc2c(N(C)CC3CCS(=O)(=O)C3)ncnc21 ; CC1OCCC1(O)Cn1cnc2c(N3CC(OCC(=O)N(C)C)C3)ncnc21 ; OCC(Cn1cnc2c(N3CCCCC3C3CCCC3O)ncnc21)C1CCOC1]

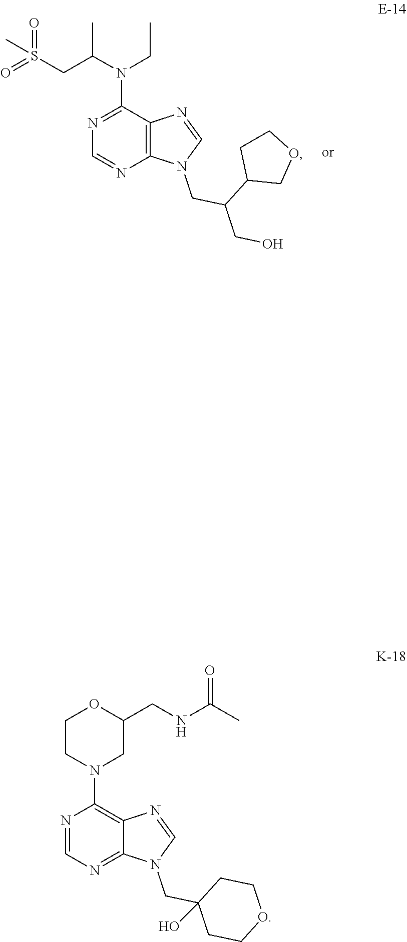 [SMILES: CCN(c1ncnc2c1ncn2CC(CO)C1CCOC1)C(C)CS(C)(=O)=O ; "or" ; CC(=O)NCC1CN(c2ncnc3c2ncn3CC2(O)CCOCC2)CCO1]

In some embodiments, a method for identifying YTH domain of YTHDF1 inhibitors is disclosed herein, the method comprising the steps of: (1) contacting an YTH domain of the YTHDF1 protein and FAM-labeled m6A RNA with a candidate inhibitor; and (2) executing FP HTS (fluorescence polarization, high-throughput screening) on the candidate inhibitor; whereby a potential inhibition provided by the candidate is indicated.

In an invention brief summary or discussion, the technology disclosed herein can be discussed by reviewing/discussing the following list of features, which can be inter-combined with any other embodiment or aspect disclosed herein:

Feature 1: A method for inhibiting a pan-YTH domain, comprising: contacting the pan-YTH domain with a small molecule pan-YTH domain inhibitor, wherein the small molecule pan-YTH domain inhibitor is capable of inhibiting multiple YTH domains found in a YTH family of proteins that recognize m6A-modified transcripts, thereby changing a post-transcriptional modification process.

Feature 2: The method of feature 1, wherein the small molecule pan-YTH domain inhibitor is a chemical compound selected from the group consisting of small organic molecules, salts thereof, tautomers thereof, hydrates thereof, and/or solvates thereof.

Feature 3: The method of feature 1, wherein the small molecule pan-YTH domain inhibitor inhibits substantially all YTH domains in the YTH family of proteins by binding to a conserved region of the YTH domains.

Feature 4: The method of feature 1, wherein the m6A-modified transcripts comprise N6-methyladenosine (m6A) modifications at consensus motifs.

Feature 5: The method of feature 1, wherein the post-transcriptional modification process is selected from the group consisting of RNA splicing, RNA stability, RNA localization, RNA translation, and RNA degradation.

Feature 6: The method of feature 1, wherein the YTH family of proteins comprises YTHDF1, YTHDF2, YTHDF3, YTHDC1, and YTHDC2.

Feature 7: The method of feature 1, wherein the inhibition of the pan-YTH domain results in a decrease in the recognition and binding of m6A-modified transcripts by the YTH family of proteins.

Feature 8: The method of feature 1, wherein the method is performed in vitro using cell-free assays.

Feature 9: A method for modulating a post-transcriptional modification process, comprising: administering a small molecule pan-YTH domain inhibitor to a cell or organism, wherein the small molecule pan-YTH domain inhibitor is capable of inhibiting multiple YTH domains found in a YTH family of proteins that recognize m6A-modified transcripts, thereby changing the post-transcriptional modification process.

Feature 10: The method of feature 9, wherein the post-transcriptional modification process is selected from the group consisting of RNA splicing, RNA stability, RNA localization, RNA translation, and RNA degradation.

Feature 11: The method of feature 9, wherein the YTH family of proteins comprises YTHDF1, YTHDF2, YTHDF3, YTHDC1, and YTHDC2.

Feature 12: The method of feature 9, wherein the cell is a mammalian cell selected from the group consisting of human cells, mouse cells, rat cells, and non-human primate cells.

Feature 13: The method of feature 9, wherein the organism is a mammal selected from the group consisting of humans, mice, rats, and non-human primates.

Feature 14: A method of treating a disease or disorder, comprising: contacting a pan-YTH domain in a human with a small molecule pan-YTH domain inhibitor, wherein the small molecule pan-YTH domain inhibitor is capable of inhibiting multiple YTH domains found in a YTH family of proteins that recognize m6A-modified transcripts, thereby changing a post-transcriptional modification process.

Feature 15: The method of feature 14, wherein the disease or disorder is selected from the group consisting of cancer, viral infections, bacterial infections, fungal infections, parasitic infections, inflammatory disorders, autoimmune disorders, neurodegenerative disorders, metabolic disorders, cardiovascular disorders, respiratory disorders, and genetic disorders.

Feature 16: The method of feature 15, wherein the cancer is selected from the group consisting of leukemia, lymphoma, melanoma, lung cancer, breast cancer, colorectal cancer, prostate cancer, ovarian cancer, and brain cancer.

Feature 17: The method of feature 15, wherein the viral infection is selected from the group consisting of HIV, hepatitis B, hepatitis C, influenza, and COVID-19.

Feature 18: The method of feature 15, wherein the inflammatory disorder is selected from the group consisting of rheumatoid arthritis, inflammatory bowel disease, psoriasis, and asthma.

Feature 19: The method of feature 15, wherein the neurodegenerative disorder is selected from the group consisting of Alzheimer's disease, Parkinson's disease, Huntington's disease, and amyotrophic lateral sclerosis.

Feature 20: The method of feature 15, wherein the metabolic disorder is selected from the group consisting of obesity, diabetes, and non-alcoholic fatty liver disease.

Feature 21: A method for inhibiting a pan-YTH domain, comprising: contacting the pan-YTH domain with a pan-YTH domain inhibitor, wherein the pan-YTH domain inhibitor is a chemical that inhibits multiple YTH domains, thereby inhibiting the pan-YTH domain and changing a post-transcriptional modification process.

Feature 22: The method of feature 21, wherein the YTH domains are found in a YTH family of proteins that recognize m6A-modified transcripts.

Feature 23: The method of feature 21, wherein the pan-YTH domain inhibitor contacts and inhibits the multiple YTH domains.

Feature 24: The method of feature 21, wherein the inhibition of the pan-YTH domain results in the changes in the post-transcriptional modification process.

Feature 25: The method of feature 21, wherein the pan-YTH domain inhibitor is a small molecule.

Feature 26: The method of feature 22, wherein the YTH family of proteins contain the YTH domains.

Feature 27: The method of feature 22, wherein the YTH family of proteins identify the m6A-modified transcripts by recognizing the m6A-modified transcripts.

Feature 28: The method of feature 27, wherein the recognition of the m6A-modified transcripts by the YTH family of proteins results in the identification of the m6A-modified transcripts.

Feature 29: The method of feature 21, wherein the pan-YTH domain inhibitor is contacted with the pan-YTH domain to inhibit the pan-YTH domain.

Feature 30: The method of feature 21, wherein the pan-YTH domain inhibitor is contacted with the pan-YTH domain to change the post-transcriptional modification process.

Feature 31: The method of feature 21, wherein the pan-YTH domain inhibitor inhibits the pan-YTH domain to change the post-transcriptional modification process.

Feature 32: The method of feature 21, wherein the pan-YTH domain inhibitor is a chemical.

Feature 33: The method of feature 32, wherein the chemical inhibits the multiple YTH domains.

Feature 34: The method of feature 21, wherein the post-transcriptional modification process is changed by the inhibition of the pan-YTH domain.

Feature 35: The method of feature 21, wherein the pan-YTH domain is inhibited by contacting with the pan-YTH domain inhibitor.

Feature 36: The method of feature 21, wherein the post-transcriptional modification process is changed by contacting the pan-YTH domain with the pan-YTH domain inhibitor.

Feature 37: The method of feature 21, wherein the pan-YTH domain inhibitor is a chemical that contacts and inhibits the multiple YTH domains.

Feature 38: The method of feature 37, wherein the contact and inhibition of the multiple YTH domains by the chemical changes the post-transcriptional modification process.

Feature 39: The method of feature 21, wherein the pan-YTH domain inhibitor is a chemical that contacts the pan-YTH domain to inhibit the pan-YTH domain and change the post-transcriptional modification process.

Feature 40: A method for changing a post-transcriptional modification process, comprising: contacting a pan-YTH domain with a pan-YTH domain inhibitor, wherein the pan-YTH domain inhibitor is a chemical that inhibits multiple YTH domains, thereby inhibiting the pan-YTH domain and changing the post-transcriptional modification process.

Feature 41: A method for identifying an YTH domain of YTHDF1 inhibitors, the method comprising the steps of: (1) contacting an YTH domain of the YTHDF1 protein and FAM-labeled m6A RNA with a candidate inhibitor; and (2) executing FP HTS (fluorescence polarization, high-throughput screening) on the candidate inhibitor; whereby a potential inhibition provided by the candidate is indicated and a lead inhibitor is identified from a pool of candidate inhibitors.

Feature 42: The method of feature 41, wherein the YTH domain of the YTHDF1 protein is a recombinant protein expressed and purified from a bacterial expression system.

Feature 43: The method of feature 41, wherein the candidate inhibitor is a small molecule compound selected from a library of diverse chemical structures of nucleoside mimetics.

Feature 44: The method of feature 41, wherein the FP HTS assay measures the competition between the candidate inhibitor and a fluorescently labeled m6A RNA binding to the YTH domain, by detecting changes in fluorescence polarization upon binding of a fluorescently labeled m6A RNA probe to the YTH domain in the presence or absence of the candidate inhibitor.

Feature 45: The method of feature 41, further comprising the step of determining the selectivity of the lead inhibitor by testing its ability to inhibit other YTH domain-containing proteins or unrelated RNA-binding proteins.

Feature 46: The method of feature 41, wherein the FP HTS assay is performed in a high-throughput format using 384-well microplates to screen a large number of candidate inhibitors simultaneously.

Other implementations are also described and recited herein. These and other features and advantages will be apparent from a reading of the following detailed description and a review of the associated drawings. It is to be understood that both the foregoing general description and the following detailed description are explanatory only and are not restrictive of aspects as claimed.

### BRIEF DESCRIPTION OF THE DRAWINGS

Solely for the purpose of illustration, certain embodiments of the present invention are explained using examples in the drawings described below. It should be understood, however, that the invention is not limited to the precise arrangements, dimensions, and configurations shown. In the figures:

FIG. 1A shows the chemical structure of m6A (left) and hydrophobic binding pocket of YTHYTHDF1 (right).

FIG. 1B shows SDS-PAGE and MS characterizations of the purified YTHYTHDF1 protein.

FIG. 1C shows 5′-FAM-GAACCGG(M6A)CUGUCUUA (SEQ ID NO: 1), 5′-FAM-GAACCGG(A)CUGUCUUA (SEQ ID NO: 2), and 5′-biotin-GAACCGG(M6A)CUGUCUUA (SEQ ID NO: 3).

FIG. 1D shows FP binding data and fitted binding curves for YTHYTHDF1 and FAM-m6A-RNA or FAM-A-RNA. In FIG. 1D the YTH-YTHDF1 protein titration is with 25 nM fluorescent m⁶A-ssRNA or A-ssRNA. Data are presented as mean±SD. FIG. 1E shows displacement of a fluorescent m⁶A-ssRNA from YTH-YTHDF1 by non-fluorescent m⁶A-ssRNA. Data are presented as mean±SD. FIG. 1E illustrates fitted IC50 value. FIG. 1F is a plot that illustrates the Z′ factor can be calculated using the following example equation [43]:

$${Z}^{′} = 1 - \frac{3 \times ( SDpos + SDneg )}{\vert Mpos - Mneg \vert}$$

Where SD is the standard deviation, and M is the mean FP value. FIG. 1F illustrates the data for the determination of Z′ factor based on N=20 repeated measurements of the FP competition assay for screening.

FIG. 2 shows screening of YTH^{YTHDF1} inhibitors by the FP competition assay. In FIG. 2 is illustrated HTS was carried out with 320 compounds from the Enamine company. Brown (or lower) point is positive control, purple (or top point) is negative control. The darker points (red points in color) are (labeled) hit compounds.

FIG. 3A shows the chemical structures of N-7 and D-6. N-7 is a pan YTH-domain inhibitor. FIG. 3B shows dose-response curves of the FP competition assay using compound N-7 or D-6 against YTHYTHDF1 protein. FIG. 3C shows dose-response curves of the FP competition assay using compound N-7 or D-6 against YTHYTHDF2 protein. FIG. 3D shows dose-response curves of the FP competition assay using compound N-7 or D-6 against YTHYTHDF3 protein. FIG. 3E shows dose-response curves of the FP competition assay using compound N-7 or D-6 against YTHYTHDC1 protein. FIG. 3F shows dose-response curves of the FP competition assay using compound N-7 or D-6 against YTHYTHDC2 protein.

FIG. 4A shows the FP binding data between N-7 and the FAM-m6A-RNA. FIG. 4B shows the results of the thermal shift assay performed on YTHYTHDF1, YTHYTHDF2, YTHYTHDF3, YTHYTHDC1, or YTHYTHDC2 proteins in the presence of compound N-7 with varying concentrations.

FIG. 5A shows the DNA sequence length information of YTHYTHDF1. Expression and purification of YTH-YTHDF1 protein is demonstrated. FIG. 5A shows the sequence information of YTH-YTHDF1. FIG. 5B shows purification of YTH-YTHDF1 protein through Nickel column under different concentrations of imidazole buffer. FIG. 5B illustrates the SDS-PAGE characterizations of the purified YTHYTHDF1 protein. FIG. 5C shows a compaison of YTH-YTHIDF1 protein purity after Nickel column and desalting column purification. FIG. 5C illustrates the SDS-PAGE characterizations of the purity of YTHYTHDF1 protein before and after buffer exchange. FIG. 5) shows a deconvoluted mass data of YTH-YTHDF1 protein.

FIG. 6A shows a comparison of the changing of total fluorescence intensity during the titration of the purified YTHYTHDF1 by FAM-m6A-RNA in the FP binding assay as the concentration of YTHYTHDF1 proteins increases. Part of the data is shown in FIG. 1D. Data are presented as mean±standard deviation (SD) with n=3 biological replicates.

FIG. 6B shows the displacement of FAM-m6A-RNA from YTHYTHDF1 by DMSO by the FP competition assay. Data are presented as mean±SD with n=2 biological replicates and up to 5% DMSO does not affect the FP signal and total fluorescence intensity of FAM-m6A-RNA.

FIG. 7A shows the chemical structures of hit compounds.

FIG. 7B shows a comparison of the FP signal change when increasing the concentration of hit compounds. FIG. 7B shows a comparison of the FP signal change when increasing the concentration of hit compounds. Data are presented as mean±SD. FIG. 7C shows a comparison of the change in relative FI as the concentration of hit compounds increases. Data are presented as mean±SD.

FIG. 8A shows a chromatogram. FIG. 8B shows a mass spectrum. FIG. 8C shows an NMR spectrum.

FIG. 9A shows the sequence information of YTH-YTHDF2.

FIG. 9B shows purification of YTH-YTHDF2 protein through Nickel column under different concentrations of imidazole buffer.

FIG. 9C shows comparison of YTH-YTHDF2 protein purity after Nickel column and desalting column purification.

FIG. 9D shows a deconvoluted mass data of YTH-YTHDF2 protein.

FIG. 10A shows DNA sequence information of YTH^{YTHDF3}. FIG. 10B shows SDS-PAGE characterizations of the purified YTH^{YTHDF3} protein. YTH^{YTHDF3} protein was purified by a Nickel column with an imidazole concentration gradient in lysis buffer. FIG. 10C shows SDS-PAGE characterizations of the purity of YTH^{YTHDF3} protein before and after buffer exchange. FIG. 10D shows full deconvolution of MS data of YTH^{YTHDF3} protein (black, observed mass; lighter grey or red, theoretical mass).

Expression and purification of YTH-YTHDC1 protein is demonstrated. FIG. 11A shows the sequence information of YTH-YTHDC1. FIG. 11B shows purifying of YTH-YTHDC1 protein through Nickel column under different concentrations of imidazole buffer. FIG. 11C shows a comparison of YTH-YTHDC1 protein purity after Nickel column and desalting column purification. FIG. 11D shows deconvoluted mass data of YTH-YTHDC1 protein.

FIG. 11A shows the sequence information of YTH-YTHDC1.

FIG. 11B shows purifying of YTH-YTHDC1 protein through Nickel column under different concentrations of imidazole buffer.

FIG. 11C shows a comparison of YTH-YTHDC1 protein purity after Nickel column and desalting column purification.

FIG. 11D shows deconvoluted mass data of YTH-YTHDC1 protein.

FIG. 12A shows the DNA sequence information of YTH^{YTHDC2}. FIG. 12B shows SDS-PAGE characterizations of the purified YTH^{YTHDC2} protein. YTHY^{THDC2} protein was purified by a Nickel column with an imidazole concentration gradient in lysis buffer.

FIG. 12C shows SDS-PAGE characterizations of the purity of YTH^{YTHDC2} protein before and after buffer exchange. FIG. 12D shows full deconvolution of MS data of YTH^{YTHDC2} protein (black, observed mass; grey or red, theoretical mass).

FIG. 13A shows FP binding data and fitted binding curves for YTH-domain proteins and FAM-m⁶A-RNA or FAM-A-RNA. Data are presented as mean±SD with n=2 or 3 biological replicates.

FIG. 13B shows the displacement of FAM-m⁶A-RNA from YTH-domain proteins by the non-fluorescent m⁶A-RNA by the FP competition assay with the fitted IC₅₀ value. Data are presented as mean±SD with n=3 biological replicates.

FIG. 14A shows dose-response curves of the FP competition assay using compound N-7 against 0.8 μM YTH^{YTHDF1}, 1.0 μM YTH^{YTHDF2}, and 0.1 μM YTH^{YTHDC1} proteins, respectively. Data are presented as mean±SD with n=3 biological replicates. FIG. 14B shows dose-response curves of the FP competition assay using FAM-m⁶A-RNA against different concentrations of N-7 and YTH^{YTHDF1}, YTH^{YTHDF2}, or YTH^{YTHDC1} proteins complex. Data are presented as mean±SD with n=3 biological replicates. FIG. 14C shows IC₅₀ values of N-7 exhibited no significant changes under different FP competition assay conditions. The two-tailed f-test was used to assess the statistical significance of the difference between the two samples, with the p-value indicated as “ns” for p≥0.05.

FIG. 15A shows an RNA electrophoretic mobility shift assay (REMSA) showing N-7's inhibitory effect on the RNA-binding activity of YTH^{YTHDF1} (left), with quantification of IC₅₀ values for N-7 inhibition of YTH^{YTHDF1} (right). FIG. 15B shows REMSA showing N-7's inhibitory effect on the RNA-binding activity of YTH^{YTHDC1} (left), with quantification of IC₅₀ values for N-7 inhibition of YTH^{YTHDC1} (right).

FIG. 16A shows SDS-PAGE characterizations of the purified FTO protein. FTO protein was purified by a Nickel column with an imidazole concentration gradient in lysis buffer. FIG. 16B shows SDS-PAGE characterizations of the purity of FTO protein before and after buffer exchange. FIG. 16C shows full deconvolution of MS data of FTO protein (black, observed mass; grey or red, theoretical mass). FIG. 16D shows FP binding data and fitted binding curves for FTO and FAM-m⁶A-RNA. Data are presented as mean SD with n=3 biological replicates. FIG. 16E shows dose-response curves of the FP competition assay using compound FB23 against FTO protein. Data are presented as mean SD with n=3 biological replicates. FIG. 16F shows dose-response curves of the FP competition assay using compound N-7 against FTO protein. Data are presented as mean SD with n=3 biological replicates.

FIG. 17 shows a characterization of binding affinity between YTH-YTHDF1 and m6A-RNA by thermal shift assay.

FIGS. 18A-18E show thermal shift assays results for the stabilization effect of N-7 against five YTH-domain proteins, including YTH^{YTHDF1} (FIG. 18A), YTH^{YTHDF2} (FIG. 18B), YTH^{YTHDF3} (FIG. 18C), YTH^{YTHDC1} (FIG. 18D), and YTH^{YTHDC2} (FIG. 18E).

FIGS. 19A-19F show thermal shift assays results for no stabilization effect of the negative control compound D-6 against five YTH-domain proteins, including YTH^{YTHDF1} (FIG. 19A), YTH^{YTHDF2} (FIG. 19B), YTH^{YTHDF3} (FIG. 19C), YTH^{YTHDC1} (FIG. 19D), and YTH^{YTHDC2} (FIG. 19E). Shown in FIG. 19F is the summary of ΔTm of different YTH domains by adding the D-6 compound.

FIG. 20A shows the chemical structure of Motif 1 (colored in grey or red) is shown on the left. The middle bar graph shows the fractions of compounds that contain Motif 1 out of all compounds in the overall library and in the hits from the screening in this study. The numbers of compounds are noted above the bar. Shown on the right are the dose-responses curves for the 6-(methylamino) purine against YTH domain-m⁶A recognition, measured by the FP competition assay.

FIG. 20B shows the chemical structure of Motif 2 (colored in grey red) is shown on the left. The bar graph shows the fractions of compounds that contain Motif 2 out of the overall library and in the hits from the screening. FIG. 20C shows the chemical structure of Motif 3 (colored in grey or red) and only one structure (N-7) contains Motif 3 in the overall library.

FIG. 21A shows a superimposition of the crystal structure of YTH^{YTHDF1}/m⁶A (PDB: 4RCJ) and the best ranked docking result of YTH^{YTHDF1} in complex with N-7.

FIG. 21B shows interactions between YTH^{YTHDF1} and N-7 by docking. m⁶A and N-7 are represented by green and cyan sticks, respectively.

FIGS. 22A-22D show FP competition assays using reported YTH domain inhibitors against all YTH domain proteins and FAM-m⁶A-RNA. Data are presented as mean±SD with n=2 or 3 biological replicates.

FIG. 22E shows a table with a Comparison of N-7 with reported YTH inhibitors based on the FP competition assay.

FIG. 23A and FIG. 23B show compounds 6 and 26 show minimal inhibition of the formation of the YTH^{YTHDC1} protein-RNA complex by the REMSA assay Given the discrepancies of IC50 values measured by the FP assay results and HTRF for compounds 6 and 26 against YTHYTHDC1, we performed orthogonal competition REMSA to confirm the minimal inhibitory activity for compounds 6 and 26 (FIG. 23A, FIG. 23B).

Any trademarks, images, likenesses, words, and depictions in the drawings and the disclosure are plainly in fair use and are provided solely for the purposes of illustration of the invention in view of an urgent need to treat subjects as further discussed in detail below. Any additional references can be included in entirety by mentioning herein.

### DETAILED DESCRIPTION OF THE INVENTION

The subject innovation is now described in some instances, when necessary, with reference to the drawings. In the following description, for purposes of explanation, numerous specific details are set forth in order to provide a thorough understanding of the present invention. It may be evident, however, that the present invention may be practiced without these specific details. In other instances, well-known structures, methods, and devices are shown in block diagram form or with illustrations in order to facilitate describing the present invention. It is to be appreciated that certain aspects, modes, embodiments, variations and features of the invention are described below in various levels of detail in order to provide a substantial understanding of the present invention.

### Definitions

For convenience, the meaning of some terms and phrases used in the specification, examples, and appended claims, are provided below. Unless stated otherwise, or implicit from context, the following terms and phrases include the meanings provided below. The definitions are provided to aid in describing particular embodiments, and are not intended to limit the claimed invention, because the scope of the invention is limited only by the claims. Unless otherwise defined, all technical and scientific terms used herein have the same meaning as commonly understood by one of ordinary skill in the art to which this invention belongs. If there is an apparent discrepancy between the usage of a term in the art and its definition provided herein, the definition provided within the specification shall prevail. In general, chemical terminology is found in the International Union of Pure and Applied Chemistry GoldBook. This disclosure is purposefully in commonly understood words, known to a person of skill in the art, but Merriam-Webster's Online Dictionary is used, when appropriate, for terms not specifically demonstrated herein or not known in the art.

As used in this specification and the appended claims, the singular forms “a,” “an” and “the” include plural referents unless the content clearly dictates otherwise. For example, reference to “a cell” includes a combination of two or more cells, and the like.

As used herein, the term “approximately” or “about” in reference to a value or parameter are generally taken to include numbers that fall within a range of 5%, 10%, 15%, or 20% in either direction (greater than or less than) of the number unless otherwise stated or otherwise evident from the context (except where such number would be less than 0% or exceed 100% of a possible value). As used herein, reference to “approximately” or “about” a value or parameter includes (and describes) embodiments that are directed to that value or parameter. For example, description referring to “about X” includes description of “X”.

As used herein, the term “or” means “and/or.” The term “and/or” as used in a phrase such as “A and/or B” herein is intended to include both A and B; A or B; A (alone); and B (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

As used herein, the term “comprising” means that other elements can also be present in addition to the defined elements presented. The use of “comprising” indicates inclusion rather than limitation. The term “including” can be interchanged with “comprising”.

The term “consisting of” refers to compositions, methods, and respective components thereof as described herein, which are exclusive of any element not recited in that description of the embodiment.

As used herein the term “consisting essentially of” refers to those elements required for a given embodiment. The term permits the presence of additional elements that do not materially affect the basic and novel or functional characteristic(s) of that embodiment of the invention. For example, a pharmaceutical formulation can consist essentially of an active agent and another active ingredient, meaning that a variety of excipients or other additives can be present in the formulation, but no other active pharmaceutical ingredient (API) is present in the formulation, except in formulations wherein an intended synergistic effect is demonstrated by the claims or examples herein (e.g., a formulation consisting essentially of one, two, or three ingredients or pharmaceutically acceptable salts thereof). In another example, a pharmaceutical formulation can consist essentially of an active agent and another ingredient, meaning that the formulation is provided in the form of a nasal spray, an inhaled formulation, an orally administered formulation, or an injection formulation, each of which is tailored for a fast-acting agent, therapeutic agent, or antidote but not tailored for long-term administration (e.g., as a simultaneous treatment). In another example, in the case of a preventative therapy to purposefully prevent a condition, the opposite, long-term combination therapy, can be referred to with “consisting essentially of”. This example could be applied to, for example, a patient who is best treated by an evolving combination therapy. In another example, the term “consisting essentially of” can also be exemplified by plain language provided in the claims.

The term “statistically significant” or “significantly” refers to statistical significance and generally means a two-standard deviation (2SD) or greater difference.

As used herein, the term “subject” refers to a mammal, including but not limited to a dog, cat, horse, cow, pig, sheep, goat, rodent, or primate. Subjects can be house pets (e.g., dogs, cats), agricultural stock animals (e.g., cows, horses, pigs, chickens, etc.), laboratory animals (e.g., mice, rats, rabbits, etc.), but are not so limited. Subjects particularly include human subjects in urgent treatment as described herein. The human subject may be a pediatric, adult, or a geriatric subject. The human subject may be of any sex.

As used herein, the terms “effective amount”, “therapeutically effective amount”, and “pharmaceutically effective amount” include an amount sufficient to prevent or ameliorate a manifestation of or a suspected manifestation of a medical condition, such as a cancer or a disease. The manifestation can be a sign or symptom or otherwise. It will be appreciated that there will be many ways known in the art to determine the effective amount for a given application. For example, the pharmacological methods for dosage determination may be used in the therapeutic context. In the context of therapeutic or prophylactic applications, the amount of a composition administered to the subject will depend on the type and severity of the medical condition and on the characteristics of the individual, such as general health, age, sex, body weight and tolerance to drugs. It will also depend on the degree, severity, and type of medical condition. The skilled artisan will be able to determine appropriate dosages depending on these and other factors. Examples of other factors can be route of administration and length of administration(s). The compositions can also be administered in combination with one or more additional therapeutic compounds.

The term “protecting group” refers to a group of atoms that, when attached to a reactive functional group in a molecule, mask, reduce or prevent the reactivity of the functional group. Typically, a protecting group may be selectively removed as desired during the course of a synthesis. Examples of protecting groups can be found in Greene and Wuts, Protective Groups in Organic Chemistry, 3^{rd} Ed. and in Harrison, et al., Compendium of Synthetic Organic Methods, Vols. 1-8. Examples of representative nitrogen protecting groups include, but are not limited to, formyl, acetyl, trifluoroacetyl, benzyl, benzyloxycarbonyl (“CBZ”), tert-butoxycarbonyl (“Boc”), trimethylsilyl (“TMS”), 2-trimethylsilyl-ethanesulfonyl (“TES”), trityl and substituted trityl groups, allyloxycarbonyl, 9-fluorenylmethyloxycarbonyl (“FMOC”), nitro-veratryloxycarbonyl (“NVOC”) and the like. Examples of representative hydroxylprotecting groups include, but are not limited to, those where the hydroxyl group is either acylated (esterified) or alkylated such as benzyl and trityl ethers, as well as alkyl ethers, tetrahydropyranyl ethers, trialkylsilyl ethers (e.g., TMS or TIPS groups), glycol ethers, such as ethylene glycol and propylene glycol derivatives and allyl ethers.

As used herein, a therapeutic that “prevents” a disorder or condition refers to a compound (or combination) that, in a statistical sample, reduces the occurrence of the disorder or condition in the treated sample relative to an untreated control sample, or delays the onset or reduces the severity of one or more symptoms of the disorder or condition relative to the untreated control sample. For example, a compound (or combination) that prevents epilepsy may reduce the frequency of seizures and/or reduce the severity of seizures.

The term “treating” includes prophylactic and/or therapeutic treatments. The term “prophylactic or therapeutic” treatment is art-recognized and includes administration to the host of one or more of the subject compositions. If it is administered prior to clinical manifestation of the unwanted condition (e.g., disease or other unwanted state of the host animal) then the treatment is prophylactic (i.e., it protects the host against developing the unwanted condition), whereas if it is administered after manifestation of the unwanted condition, the treatment is therapeutic, (i.e., it is intended to diminish, ameliorate, or stabilize the existing unwanted condition or side effects thereof).

The phrases “conjoint administration” and “administered conjointly” refer to any form of administration of two or more different therapeutic compounds such that the second compound is administered while the previously administered therapeutic compound is still effective in the body (e.g., the two compounds are simultaneously effective in the patient, which may include synergistic effects of the two compounds). For example, the different therapeutic compounds can be administered either in the same formulation or in a separate formulation, either concomitantly or sequentially. In certain embodiments, the different therapeutic compounds can be administered at the same time, within one minute, 2 minutes, 4 minutes, 6 minutes, 10 minutes, 30 minutes, or an hour or 90 minutes of one another. In some embodiments, the different therapeutic compounds can be administered within 1 year of one another. Thus, an individual who receives such treatment can benefit from a combined effect of different therapeutic compounds.

As used herein, the terms “treat,” “treatment,” “treating,” or “amelioration” when used in reference to a disease, disorder, or medical condition, refer to therapeutic treatments for a condition, wherein the object is to reverse, alleviate, ameliorate, inhibit, slow down or stop the progression or severity of a symptom or condition. The term “treating” includes reducing or alleviating at least one adverse effect or symptom of a condition. Treatment is generally “effective” if one or more symptoms or clinical markers are reduced. Alternatively, treatment is “effective” if the progression of a condition is reduced or halted. That is, “treatment” includes not just the improvement of symptoms or markers, but also a cessation or at least slowing of progress or worsening of symptoms that would be expected in the absence of treatment. Beneficial or desired clinical results include, but are not limited to, alleviation of one or more symptom(s), sign(s), diminishment of extent of the deficit, stabilized (i.e., not worsening) state of a symptom or condition, delay or slowing of onset of symptoms or indications, and an increased lifespan as compared to that expected in the absence of treatment.

As used herein, the term “long-term” administration means that the therapeutic agent or drug is administered for a period of at least 12 weeks. This includes that the therapeutic agent, combination, or drug is administered such that it is effective over, or for, a period of at least 12 weeks and does not necessarily imply that the administration itself takes place for 12 weeks, e.g., if sustained release compositions or long-acting therapeutic agent or drug is used. Thus, the subject is treated for a period of at least 12 weeks. In many cases, long-term administration is for at least 4, 5, 6, 7, 8, 9 months or more, or for at least 1, 2, 3, 5, 7 or 10 years, or more.

The administration of the compositions contemplated herein may be carried out in any convenient manner, including by aerosol inhalation, injection, ingestion, transfusion, implantation, application (e.g., topical, otic, or ocular), or transplantation. Administration can be accomplished by an implant. In some embodiments, compositions are administered parenterally. The phrases “parenteral administration” and “administered parenterally” as used herein refers to modes of administration other than enteral and topical administration, usually by injection, and includes, without limitation, intravascular, intravenous, intramuscular, intraarterial, intrathecal, intracapsular, intraorbital, intratumoral, intracardiac, intradermal, intraperitoneal, transtracheal, subcutaneous, subcuticular, intraarticular, subcapsular, subarachnoid, intraspinal and intrasternal injection and infusion. In one embodiment, the compositions contemplated herein are administered to a subject by direct injection into an artery, vein, lymph node, or organ (e.g., heart, muscle, organ).

The terms: “decrease”, “reduced”, “reduction”, or “inhibit” are all used herein to mean a decrease by a statistically significant amount. In some embodiments, “reduce,” “reduction” or “decrease” or “inhibit” typically means a decrease by at least 10% as compared to a reference level (e.g., the absence of a given treatment or agent) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass a complete inhibition or reduction as compared to a reference level. “Complete inhibition” is a 100% inhibition as compared to a reference level. A decrease can be preferably down to a level accepted as within the range of normal for an individual without a given disorder.

The term “prodrug” is intended to encompass compounds which, under physiologic conditions, are converted into the therapeutically active agents of the present invention (e.g., any compound selected from this disclosure). A common method for making a prodrug is to include one or more selected moieties which are hydrolyzed under physiologic conditions to reveal the desired molecule. In other embodiments, the prodrug is converted by an enzymatic activity of the host animal. For example, esters or carbonates (e.g., esters or carbonates of alcohols or carboxylic acids) are preferred prodrugs of the present invention. In certain embodiments, some or all of the compounds selected from this disclosure in a formulation can be replaced with the corresponding suitable prodrug, e.g., wherein a hydroxyl in the parent compound is presented as an ester or a carbonate or carboxylic acid present in the parent compound is presented as an ester. In some embodiments, a “prodrug” is made by using an absorbing particle that subsequently releases an active form after administration.

In some embodiments, the decrease in the one or more signs or symptoms is evaluated according to the DSM-5. In some embodiments, signs are observed or measured by a health care provider. Symptoms can be reported by the subject. In some embodiments, the decrease of signs or symptoms occurs in less than about 120 minutes, 90 minutes, less than about 60 minutes, less than about 30 minutes, less than about 15 minutes, less than about 10 minutes, or less than about 5 minutes, or less than about 3 minutes, or less than about 1 minute. In some embodiments, the decrease of signs or symptoms occurs in less than 1 day, less than 1 week, less than 1 month, or in less than 1 year.

The terms “increased”, “increase”, “enhance”, or “activate” are all used herein to mean an increase by a statically significant amount. In some embodiments, the terms “increased”, “increase”, “enhance”, or “activate” can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level. In the context of a marker or symptom, a “increase” is a statistically significant increase in such level.

As used herein, an agent or a therapeutic agent provided to a subject and suspected to be or involved in a treatment can be a small molecule less than 1000 MW or a large molecule not less than 1000 MW including, for example, biologics, oligonucleotides, peptides, systems of large molecules, oligosaccharides, and larger molecules. Any of the therapeutic agents disclosed herein can be used as or in combination with small molecules and/or large molecules as discussed herein.

As used herein, a subject may or may not be aware of suffering from a cancer or a disease condition. A health care provider may suspect a disease or cancer or may have confirmed cancer or disease.

A subject can be one who has been previously diagnosed with or identified as suffering from or having a condition in need of treatment (e.g., a cancer or related disorder) or one or more complications related to such a condition, and optionally, but need not have already undergone treatment for a condition or the one or more complications related to the condition. Alternatively, a subject can also be one who has not been previously diagnosed as having a condition in need of treatment or one or more complications related to such a condition. For example, a subject can be one who exhibits one or more risk factors for a condition, or one or more complications related to a condition or a subject who does not exhibit risk factors. A “subject in need” of treatment for a particular condition can be a subject having that condition, diagnosed as having that condition, suspected as having, or at risk of developing that condition. In another example, the subject has been brought into a treatment situation entirely without the subject's knowledge and/or intent. For example, a subject can obviously be in need of treatment but not be responsive to a treatment, and as described herein the present methods and formulations may be used to help save the subject's life.

Pharmaceutical Compositions: The compositions and methods of the present invention may be utilized to prevent a need for other treatment, to provide benefit when other treatment(s) fail, or to treat an individual in need thereof. In some embodiments, the individual is suspected of needing treatment. In certain embodiments, the individual is a mammal such as a human, or a non-human mammal. When administered to an animal, such as a human, the composition or the compound is preferably administered as a pharmaceutical composition comprising, for example, a compound of the invention and a pharmaceutically acceptable carrier.

Pharmaceutically acceptable carriers are well known in the art and include, for example, aqueous solutions such as water or physiologically buffered saline or other solvents or vehicles such as glycols, glycerol, oils such as olive oil, or injectable organic esters. In some embodiments, when such pharmaceutical compositions are for human administration, particularly for invasive routes of administration (i.e., routes, such as injection or implantation, that circumvent transport or diffusion through an epithelial barrier), the aqueous solution is pyrogen-free, or substantially pyrogen-free. The excipients can be chosen, for example, to effect delayed release of an agent or to selectively target one or more cells, tissues, or organs. The pharmaceutical composition can be in dosage unit form such as tablet, capsule (including sprinkle capsule and gelatin capsule), granule, lyophile for reconstitution, powder, solution, syrup, suppository, injection or the like. The composition can also be present in a transdermal delivery system, e.g., a skin patch. The composition can also be present in a solution suitable for topical administration, such as a lotion, cream, or ointment.

A pharmaceutically acceptable carrier can contain physiologically acceptable agents that act, for example, to stabilize, increase solubility or to increase the absorption of a compound such as a compound of the invention. Such physiologically acceptable agents include, for example, carbohydrates, such as glucose, sucrose or dextrans, antioxidants, such as ascorbic acid or glutathione, chelating agents, low molecular weight proteins or other stabilizers or excipients. The choice of a pharmaceutically acceptable carrier, including a physiologically acceptable agent, depends, for example, on the route of administration of the composition. The preparation or pharmaceutical composition can be a self-emulsifying drug delivery system or a self-micro emulsifying drug delivery system. The pharmaceutical composition (preparation) also can be a liposome or other polymer matrix, which can have incorporated therein, for example, a compound of the invention. Liposomes, for example, which comprise phospholipids or other lipids, are nontoxic, physiologically acceptable and metabolizable carriers that are relatively simple to make and administer.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible compositions employed in pharmaceutical formulations.

A pharmaceutical composition (preparation) can be administered to a subject by any of a number of routes of administration including, for example, orally (for example, drenches as in aqueous or non-aqueous solutions or suspensions, tablets, capsules (including sprinkle capsules and gelatin capsules), boluses, powders, granules, pastes for application to the tongue); absorption through the oral mucosa (e.g., sublingually); subcutaneously; transdermally (for example as a patch applied to the skin); and topically (for example, as a cream, ointment or spray applied to the skin). The compound may also be formulated for inhalation. Inhalation can include inhalation of a liquid (droplets or aerosol). Inhalation can include a micronized powder adhered to carrier particles or can be without carrier particles. In certain embodiments, a compound may be simply dissolved or suspended in sterile water. Details of appropriate routes of administration and compositions suitable for same can be found in, for example, U.S. Pat. Nos. 6,110,973, 5,763,493, 5,731,000, 5,541,231, 5,427,798, 5,358,970 and 4,172,896, as well as in patents cited therein.

The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient that can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association an active compound, such as a compound of the invention, with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules (including sprinkle capsules and gelatin capsules), cachets, pills, tablets, lozenges (using a flavored basis, usually sucrose and acacia or tragacanth), lyophile, powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. Compositions or compounds may also be administered as a bolus, electuary or paste.

To prepare solid dosage forms for oral administration (capsules, including sprinkle capsules and gelatin capsules), tablets, pills, dragées, powders, granules and the like), the active ingredient is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof, (10) complexing agents, such as, modified and unmodified cyclodextrins; and (11) coloring agents. In the case of capsules (including sprinkle capsules and gelatin capsules), tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropyl methyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions, such as dragées, capsules (including sprinkle capsules and gelatin capsules), pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropyl methyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions that can be used include polymers and waxes. The active ingredient can also be in micro-encapsulated form, if appropriate, with one or more of the above-described excipients.

Liquid dosage forms useful for oral administration include pharmaceutically acceptable emulsions, lyophiles for reconstitution, micro-emulsions, solutions, suspensions, syrups and elixirs. In addition to the active ingredient, the liquid dosage forms may contain inert diluents commonly used in the art, such as, for example, water or other solvents, cyclodextrins and derivatives thereof, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Dosage forms for the topical or transdermal administration include powders, sprays, ointments, pastes, creams, lotions, gels, solutions, patches and inhalants. The active compound may be mixed under sterile conditions with a pharmaceutically acceptable carrier, and with any preservatives, buffers, or propellants that may be required.

The ointments, pastes, creams and gels may contain, in addition to an active compound, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to an active compound, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Such dosage forms can be made by dissolving or dispersing the active compound in the proper medium. Absorption enhancers can also be used to increase the flux of the compound across the skin. The rate of such flux can be controlled by either providing a rate controlling membrane or dispersing the compound in a polymer matrix or gel.

The phrases “parenteral administration” and “administered parenterally” as used herein means modes of administration other than enteral and topical administration, usually by injection, and includes, without limitation, intravenous, intraocular (such as intravitreal), intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion. Pharmaceutical compositions suitable for parenteral administration comprise one or more active compounds in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers that may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Examples of suitable aqueous and nonaqueous carriers that may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which, in turn, may depend upon crystal size and crystalline form. Alternatively, delayed absorption of a parenterally administered drug form is accomplished by dissolving or suspending the drug in an oil vehicle.

Injectable depot forms are made by forming microencapsulated matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissue.

For use in the methods of this invention, active compounds can be given per se or as a pharmaceutical composition containing, for example, 0.1 to 99.5% (more preferably, 0.5 to 90%) of active ingredient in combination with a pharmaceutically acceptable carrier.

Methods of introduction may also be provided by rechargeable or biodegradable devices. Various slow-release polymeric devices have been developed and tested in vivo in recent years for the controlled delivery of drugs, including proteinaceous biopharmaceuticals. A variety of biocompatible polymers (including hydrogels), including both biodegradable and non-degradable polymers, can be used to form an implant for the sustained release of a compound at a particular target site.

Actual dosage levels of the active ingredients in the pharmaceutical compositions may be varied so as to obtain an amount of the active ingredient that is effective to achieve the desired therapeutic response for a particular patient, composition, and mode of administration, without being toxic to the patient.

The selected dosage level will depend upon a variety of factors including the activity of the particular compound or combination of compounds employed, or the ester, salt or amide thereof, the route of administration, the time of administration, the rate of excretion of the particular compound(s) being employed, the duration of the treatment, other drugs, compounds and/or materials used in combination with the particular compound(s) employed, the age, sex, weight, condition, general health and prior medical history of the patient being treated, and like factors well known in the medical arts.

A physician or veterinarian having ordinary skill in the art can readily determine and prescribe the therapeutically effective amount of the pharmaceutical composition required. For example, the physician or veterinarian could start doses of the pharmaceutical composition or compound at levels lower than that required in order to achieve the desired therapeutic effect and gradually increase the dosage until the desired effect is achieved. By “therapeutically effective amount” is meant the concentration of a compound that is sufficient to elicit the desired therapeutic effect. It is generally understood that the effective amount of the compound will vary according to the weight, sex, age, and medical history of the subject. Other factors which influence the effective amount may include, but are not limited to, the severity of the patient's condition, the disorder being treated, the stability of the compound, and, if desired, another type of therapeutic agent being administered with the compound of the invention. A larger total dose can be delivered by multiple administrations of the agent. Methods to determine efficacy and dosage are known to those skilled in the art. See, e.g., Isselbacher, et al., (1996).

In general, a suitable daily dose of an active compound used in the compositions and methods of the invention will be that amount of the compound that is the lowest dose effective to produce a therapeutic effect. Such an effective dose will generally depend upon the factors described above.

If desired, the effective daily dose of the active compound may be administered as one, two, three, four, five, six or more sub-doses administered separately at appropriate intervals throughout the day, optionally, in unit dosage forms. In certain embodiments of the present invention, the active compound may be administered two or three times daily. In other embodiments, the active compound will be administered once daily.

The subject or patient receiving this treatment is any animal in need, including primates, in particular humans; and other mammals such as equines bovine, porcine, sheep, feline, and canine; poultry; and pets in general.

In certain embodiments, compounds of the invention may be used alone or conjointly administered with another type of therapeutic agent.

The present disclosure includes the use of pharmaceutically acceptable salts of compounds of the invention in the compositions and methods of the present invention. In certain embodiments, contemplated salts of the invention include, but are not limited to, alkyl, dialkyl, trialkyl or tetra-alkyl ammonium salts. In certain embodiments, contemplated salts of the invention include, but are not limited to, L-arginine, benenthamine, benzathine, betaine, calcium hydroxide, choline, deanol, diethanolamine, diethylamine, 2-(diethylamino)ethanol, ethanolamine, ethylenediamine, N-methylglucamine, hydrabamine, 1H-imidazole, lithium, L-lysine, magnesium, 4-(2-hydroxyethyl)morpholine, piperazine, potassium, 1-(2-hydroxyethyl)pyrrolidine, sodium, triethanolamine, tromethamine, and zinc salts. In certain embodiments, contemplated salts of the invention include, but are not limited to, Na, Ca, K, Mg, Zn or other metal salts. In certain embodiments, contemplated salts of the invention include, but are not limited to, 1-hydroxy-2-naphthoic acid, 2,2-dichloroacetic acid, 2-hydroxyethanesulfonic acid, 2-oxoglutaric acid, 4-acetamidobenzoic acid, 4-aminosalicylic acid, acetic acid, adipic acid, 1-ascorbic acid, 1-aspartic acid, benzenesulfonic acid, benzoic acid, (+)-camphoric acid, (+)-camphor-10-sulfonic acid, capric acid (decanoic acid), caproic acid (hexanoic acid), caprylic acid (octanoic acid), carbonic acid, cinnamic acid, citric acid, cyclamic acid, dodecylsulfuric acid, ethane-1,2-disulfonic acid, ethanesulfonic acid, formic acid, fumaric acid, galactaric acid, gentisic acid, d-glucoheptonic acid, d-gluconic acid, d-glucuronic acid, glutamic acid, glutaric acid, glycerophosphoric acid, glycolic acid, hippuric acid, hydrobromic acid, hydrochloric acid, isobutyric acid, lactic acid, lactobionic acid, lauric acid, maleic acid, 1-malic acid, malonic acid, mandelic acid, methanesulfonic acid, naphthalene-1,5-disulfonic acid, naphthalene-2-sulfonic acid, nicotinic acid, nitric acid, oleic acid, oxalic acid, palmitic acid, pamoic acid, phosphoric acid, proprionic acid, l-pyroglutamic acid, salicylic acid, sebacic acid, stearic acid, succinic acid, sulfuric acid, 1-tartaric acid, thiocyanic acid, p-toluenesulfonic acid, trifluoroacetic acid, and undecylenic acid salts. The HCl can be replaced by other pharmaceutically acceptable cation(s) and/or anion(s) or the methyl ester can be removed/cleaved.

The pharmaceutically acceptable acid addition salts can also exist as various solvates, such as with water, methanol, ethanol, dimethylformamide, and the like. Mixtures of such solvates can also be prepared. The source of such solvate can be from the solvent of crystallization, inherent in the solvent of preparation or crystallization, or adventitious to such solvent. The invention contemplates polymorphs, cocrystals, and amorphous forms of all substances discussed herein. As discussed above, solvates and/or hydrates can be formed by, for example, a slow evaporation whereby water and/or solvent remain hydrogen bonded with OH groups in the molecule. The formation of a solvate/hydrate can be quickly confirmed after the evaporation by using attenuated total reflectance Fourier transform infra-red spectroscopy wherein the solid solvate/hydrate is directly placed on the instrument and the subsequent IR spectrum is compared to the IR spectrum of the solid non-solvate, non-hydrate.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: (1) water-soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal-chelating agents, such as citric acid, ethylenediamine tetraacetic acid (EDTA), sorbitol, tartaric acid, phosphoric acid, and the like.

Unless otherwise defined herein, scientific and technical terms used in connection with the present application shall have the meanings that are commonly understood by those of ordinary skill in the art to which this disclosure belongs. It should be understood that this invention is not limited to the particular methodology, protocols, and reagents, etc., described herein and as such can vary. The terminology used herein is for the purpose of describing particular embodiments only and is not intended to limit the scope of the present invention, which is defined solely by the claims. Definitions of common terms in immunology and molecular biology can be found in The Merck Manual of Diagnosis and Therapy; The Encyclopedia of Molecular Cell Biology and Molecular Medicine; Molecular Biology and Biotechnology: a Comprehensive Desk Reference; Immunology; Janeway's Immunobiology; Lewin's Genes XI; Molecular Cloning: A Laboratory Manual; Basic Methods in Molecular Biology; Laboratory Methods in Enzymology; Current Protocols in Molecular Biology (CPMB); Current Protocols in Protein Science (CPPS); and Current Protocols in Immunology (CPI).

In the embodiments discussed and in any of the aspects, the disclosure described herein does not concern a process for cloning human beings, processes for modifying the germ line genetic identity of human beings, uses of human embryos for industrial or commercial purposes or processes for modifying the genetic identity of animals which are likely to cause them suffering without any substantial medical benefit to man or animal, and also animals resulting from such processes.

Other terms are defined herein within the description of the various aspects of the invention.

### Discovery of a New Inhibitor for Yth Domain-Containing M6a RNA Readers

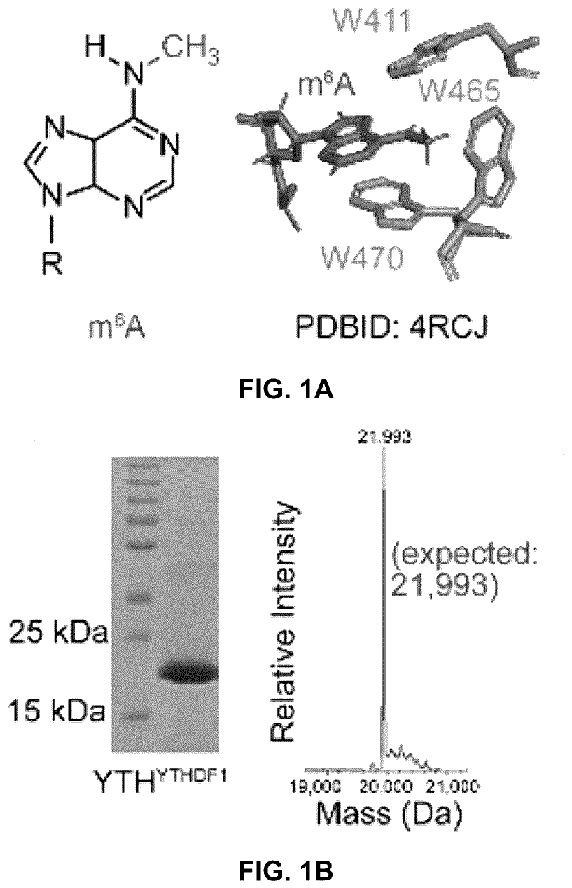

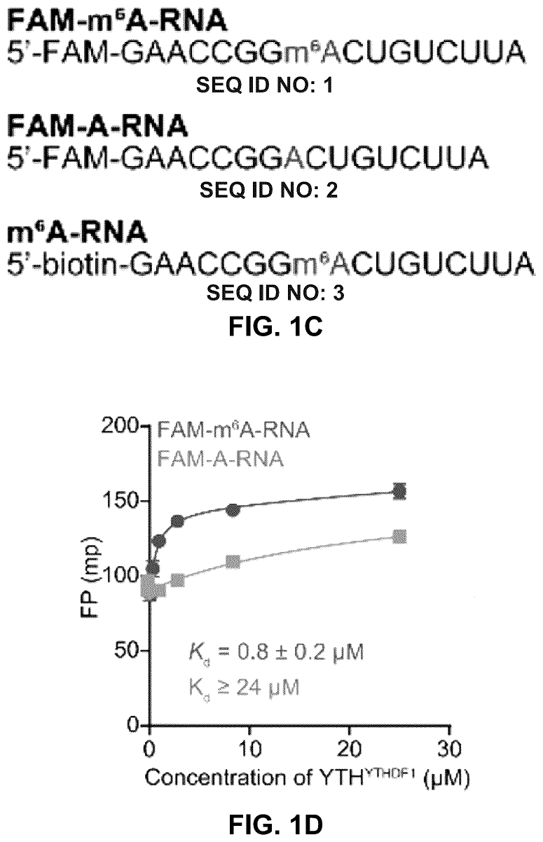

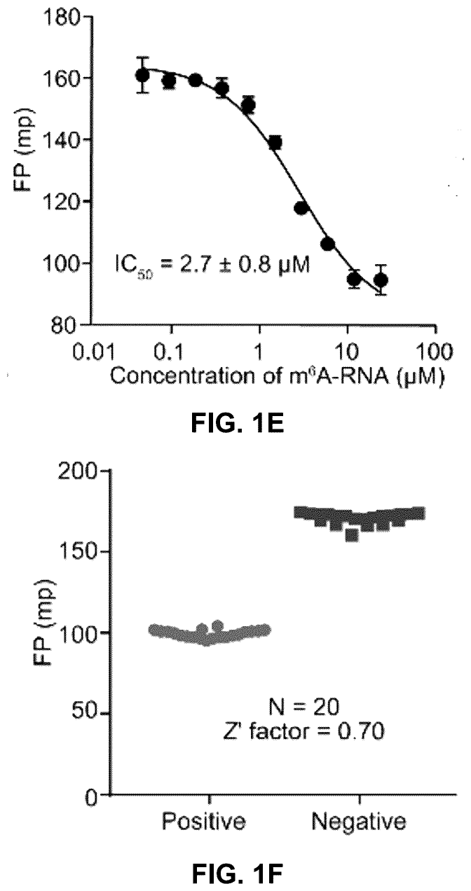

Introduction: N⁶-methyladenosine (m⁶A) is one of the most abundant internal chemical modifications in eukaryotic messenger RNAs (mRNAs)[1-4] and exerts important regulatory functions in mRNA metabolism and gene expression, primarily mediated through direct or indirect recognition by m⁶A “readers” [5-13]. The m⁶A “readers” refer to RNA binding proteins that bind to m⁶A-modified RNAs with much greater affinity to unmodified RNAs[14, 15]. The YT521-B homology (YTH) family proteins, including YTHDF1-3, YTHDC1, and YTHDC2, are the first reported and the most extensively studied m⁶A readers[3, 11]. YTH family proteins contain a YTH domain at or close to the C-termini of the full-length protein, which is responsible for m⁶A RNA recognition (e.g., FIG. 1A). In vitro binding studies showed that the purified YTH domains from YTHDF1-3, and YTHDC1 bind m⁶A-modified RNA oligonucleotides with micromolar binding affinities that are 10-100 fold tighter than binding with unmodified RNAs that carry the same sequences[16, 17]. High-resolution structures of the recognition complexes between m⁶A RNA and the YTH domains studies revealed that YTH domains directly recognize m⁶A through a hydrophobic binding pocket that contained two or three tryptophan residues (FIG. 1A); additionally, the YTH domain also presents a cleft that is rich in basic residues that present electrostatic interactions with the RNA backbone[15, 16, 18-23].

In some embodiments, this disclosure presents the first time that anyone uses the YTH domain (residues 375-552) of the human YTHDF1 protein in this assay. The experimental procedures on how we execute the FP HTS are novel compared to any existing YTH-m6A FP assays. Increasing details will now be disclosed.

Alongside with the m⁶A recognition by the YTH domain, the rest regions of the YTH family proteins are involved in sophisticated protein-protein interactions in cells and exert diverse functional consequences of m⁶A RNA recognition by readers. For example, Du et al. showed the N-terminal domain of human YTHDF2 protein directly interacts with the scaffolding subunit CNOT1 in the CCR4-NOT deadenylase complex via co-immunoprecipitation assays, which promotes the deadenylation-dependent mRNA decay[23]. Such interaction accelerates the decay of m⁶A-modified mRNAs. Meanwhile, the N-terminal domain of YTHDF2 was reported to interact with other proteins including the heat-responsive protein 12 (HRSP12) and the upstream frameshift 1 (UPF1) protein[19, 22]. Both interactions between YTHDF2 and HRSP12 or UPF1 promote the decay of m⁶A-modified mRNAs through internal cleavage or nonsense-mediated RNA decay, respectively[19, 22]. Besides YTHDF2, functions of YTH family proteins are involved in most biological transactions, including RNA decay[8], nuclear export[10], RNA splicing[21], translation[7, 8], transcription[5], the formation and functions of large ribonucleoprotein complex assemblies such as stress granules and P bodies[18, 20, 23, 24].

In addition to the intricate protein-protein interaction network YTH proteins get involved in, the YTH domains were reported to recognize substrates beyond m⁶A RNA in vitro and in cellulo, including m¹A[25, 26] and N⁶-methyldeoxyadenosine (6 mA) in DNA[27]. All these factors complicate the studies of the precise functional roles of YTH family proteins and chemical modifications in physiology and diseases. Indeed, dysregulated expression levels of YTH proteins frequently occur in embryonic development[5], neuronal development[28], memory formation[29], multiple aspects of cancer biology[30, 31], and viral infections[32]. The current experimental strategies to study the biological consequences of YTH family proteins primarily rely on the knockdown, knockout, or overexpression of a YTH protein of interest[33, 34]. Such perturbations will concurrently interrupt all interactions the protein may have, making it inconclusive to reason whether the YTH-m⁶A recognition is causative for the observed biological consequence.

The development of novel small molecule inhibitors that specifically perturb the YTH-m⁶A recognition will provide invaluable insights to dissect the sophisticated interaction network YTH proteins involve and reveal m⁶A-causitive YTH functions. Indeed, a recent report by Zou et al identified a natural product salvianolic acid C (SAC) as a selective inhibitor for m⁶A-RNA and YTH^{YTHDF1} with a 20-fold higher observed inhibitory effect compared to YTH^{YTHDF2} via AlphaScreen assays in vitro[35], although the molecular mechanism of the observed selectivity remains to be characterized. SAC contributed to elucidating the function of YTHDF1 in the formation of ribonucleoprotein condensations and translation regulation in neurons[35]. Aside from SAC, only a handful of other inhibitors have been reported for YTH and m⁶A recognition[36-42]. It remains in great demand to explore broader chemical space for inhibitor discoveries and to carry out in-depth characterizations of inhibitor selectivity against different YTH family proteins.

Here we report the discovery of an inhibitor for m⁶A-YTH recognition, resulted from the screening of a commercially available nucleoside analogue library against the YTH domain of YTHDF1 protein. Very interestingly, our screening data identified lead compound structures resembling adenosine, with modifications on the ribose moiety and the N6 position. We chose to further characterize a representative compound N-7, which showed apan-inhibitory effect of m⁶A RNA-YTH recognition for five YTH domains of YTHDF1, YTHDF2, YTHDF3, YTHDC1, and YTHDC2 proteins with IC₅₀ ranging from 30-48 μM. We demonstrated that the inhibitory activity results from the direct interaction between N-7 and the YTH domain proteins via thermal shift assay. We systematically compared the inhibitory activity and selectivity against different YTH domains of N-7 to four reported YTH domain inhibitors (SAC[35], m⁶A nucleoside[36, 37], compound 6[36], and compound 26[38]). The comparison revealed that N-7 showed competitive inhibitory activity among the reported inhibitors and presented unique pan-inhibitory activity against the five YTH domains.

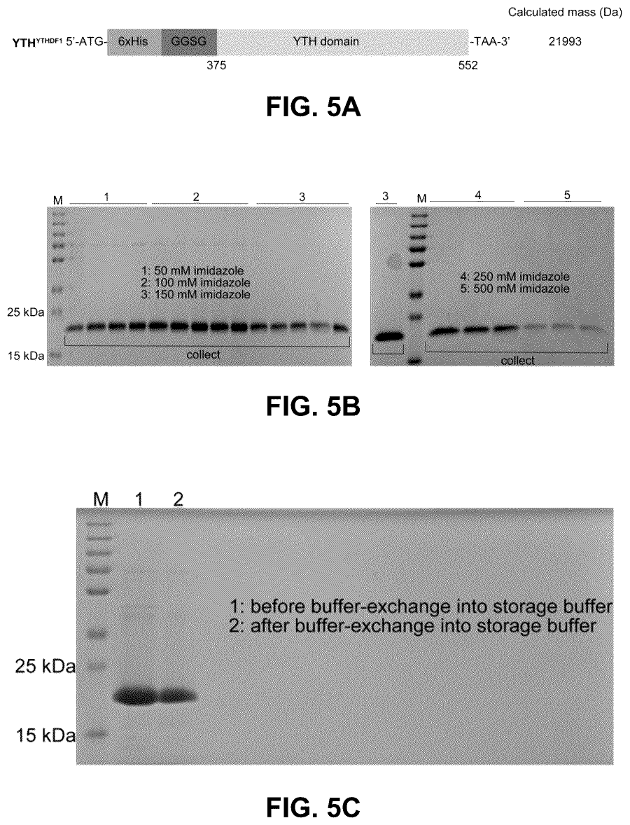

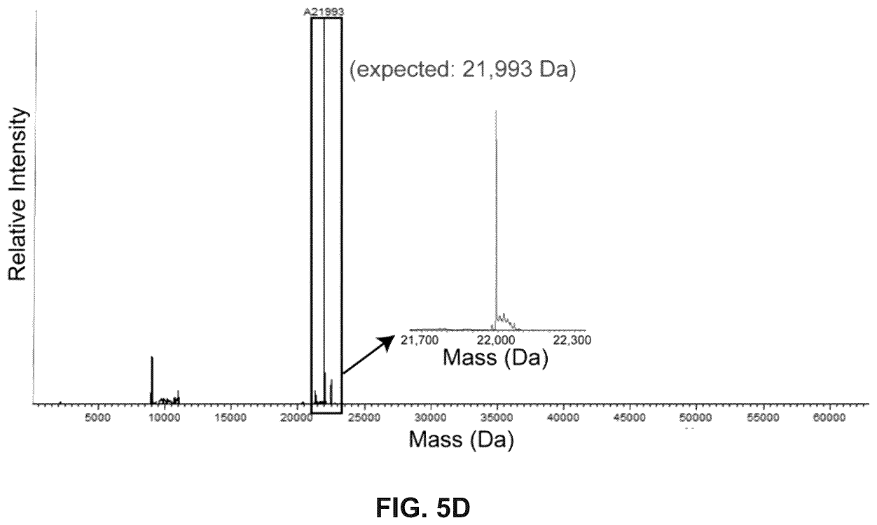

Results and Discussion Screening of inhibitors for YTH^{YTHDF}1-m⁶A RNA recognition: Firstly, we confirmed that the prepared YTH domain of YTHDF1 (noted as YTH^{YTHDF1}) was an m⁶A reader by measuring the binding affinities of the protein with the synthetic oligonucleotides via the fluorescence polarization (FP) binding assay. We over-expressed and purified YTH^{YTHDF1} protein with an N-terminal His tag from E. coli cells, and validated the size of the purified protein via SDS-PAGE and mass spectrometry (MS) (FIG. 1B, ESI, and FIG. 5A). The purified YTH^{YTHDF1} showed binding to the 16-nucleotide (nt) FAM-labeled m⁶A-containing oligonucleotide (“FAM-m⁶A-RNA”) with K_{d}=0.8±0.2 μM measured by the FP binding assay (FIG. 1C and FIG. 1D), consistent with the previously reported affinity (K_{d}=1.0±0.3 μM) measured by the isothermal titration calorimetry (ITC)[15].

FIG. 1C shows 5′-FAM-GAACCGG(M6A)CUGUCUUA (SEQ ID NO: 1), 5′-FAM-GAACCGG(A)CUGUCUUA (SEQ ID NO: 2), and 5′-biotin-GAACCGG(M6A)CUGUCUUA (SEQ ID NO: 3).

TABLE 1
SEQUENCES ILLUSTRATED IN FIG. 1C

| SEQ Number: | SEQ Description: |
|---|---|
| SEQ ID NO: 1 | 5′-FAM-GAACCGG(M6A)CUGUCUUA |
| SEQ ID NO: 2 | 5′-FAM-GAACCGG(A)CUGUCUUA |
| SEQ ID NO: 3 | 5′-biotin-GAACCGG(M6A)CUGUCUUA |

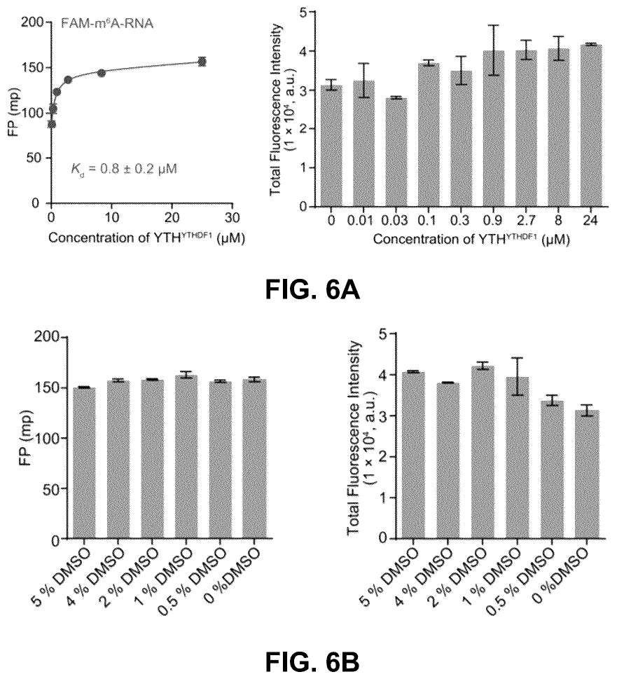

Meanwhile, we observed significantly weakened binding (K_{d}≥24 μM) between the YTH^{YTHDF1} and unmodified RNA with the same sequence (“FAM-RNA”) by the FP assay (FIG. 1C and FIG. 1D). During the titration of the purified YTH^{YTHDF1} up to 24 μM in the FP binding assay, the total fluorescence intensities (i.e. the sum of the parallel and perpendicular fluorescence intensities) produced by the FAM-labeled RNA oligonucleotides remain stable (FIG. 6A, ESI), suggesting the FP assay is a suitable assay to quantify the binding between the used RNA oligonucleotides and the YTH domain.

The screening of YTH^{YTHDF1} inhibitors by FP competition assay is illustrated. FIG. 1A shows the chemical structure of m⁶A (left) and hydrophobic binding pocket of YTH^{YTHDF1} (right). FIG. 1B shows SDS-PAGE and MS characterizations of the purified YTH^{YTHDF1} protein. Shown are the gel image of the purified protein with the Coomassie Blue staining (left) and the deconvolution mass data and the theoretical expected mass (in grey or red) of the purified protein.

To identify small molecule inhibitors of YTH-domain proteins, we set up an FP competition assay with the purified YTH^{YTHDF1} for the screening. Briefly, 25 nM of FAM-m⁶A-RNA was pre-incubated with 2 μM (greater than 2 times of the measured K_{d}) YTH^{YTHDF1}; an increasing concentration of a non-fluorescent “m⁶A-RNA” (FIG. 1C) was titrated into the pre-formed FAM-m⁶A-RNA:YTH^{YTHDF1} complex, to compete binding. When the FAM-m⁶A-RNA got competed off the RNA-protein complex, it became free RNA with reduced FP. As expected, we observed FP decreased upon increasing concentrations of the non-fluorescent m⁶A-RNA, with an IC₅₀=2.7±0.8 μM (FIG. 1E). Next, we used the non-fluorescent m⁶A-RNA and blank binding buffer as the “positive control” and “negative control” competitors to perform the FP competition assay for 20 repeats and the repeated assays generated a Z′ factor=0.7[43], demonstrating the excellent performance of the FP competition assay for screening (FIG. 1F). Additionally, we confirmed that the FP signal was not affected by the addition of 5% DMSO as small molecule libraries were dissolved in 100% DMSO (FIG. 6B, ESI).

FIG. 1C shows sequences of three RNA oligonucleotides: FAM-m⁶A-RNA, FAM-A-RNA, and m⁶A-RNA were used in the FP assay. FIG. 1D shows FP binding data and fitted binding curves for YTH^{YTHDF1} and FAM-m⁶A-RNA or FAM-A-RNA. Data are presented as mean±standard deviation (SD) with n=3 biological replicates. FIG. 1E shows the displacement of FAM-m⁶A-RNA from YTH^{YTHDF1} by the non-fluorescent m⁶A-RNA by the FP competition assay with the fitted IC₅₀ value. Data are presented as mean±SD with n=3 biological replicates. FIG. 1F shows the data for the determination of Z′ factor based on N=20 repeated measurements of the FP competition assay for screening.

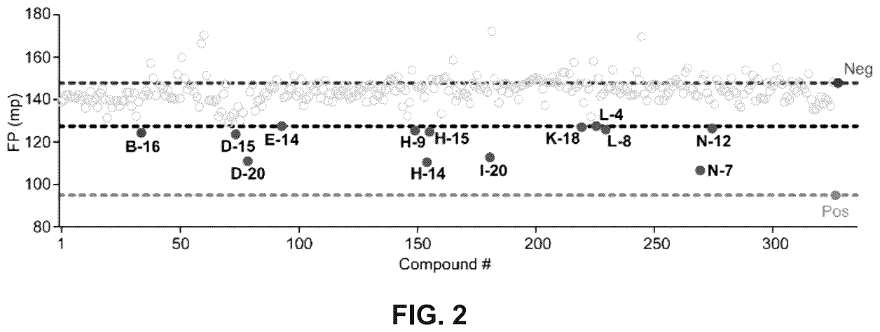

We conducted screening of 320 compounds from a commercially available nucleoside analogue library with the FP competition assay; excitingly, we identified several compounds showing reduced FP relative to the DMSO control when added to the pre-formed FAM-m⁶A-RNA:YTH^{YTHDF1} complex at the final compound concentration of 40 μM during the screening, suggesting them as potential binding inhibitors (FIG. 2). Next, we followed up with the top 13 compounds that showed at least a 35% reduction of the FP signal relative to the DMSO control (see FIG. 2).

FIG. 2 shows screening of YTH^{YTHDF1} inhibitors by the FP competition assay. Shown are the data of the screening against 320 compounds from the nucleoside mimetics library. The concentration of each compound is 40 μM with two technical replicates. The FP values measured for the positive (“m⁶A-RNA”) and negative controls (“DMSO”) are shown in brown (lighter, lower “Pos” line) and purple (upper, “Neg” line). Compounds are shown in red (i.e., solid darker circles) if they show at least a 35% reduction of FP (cut-off indicated by the black dashed line) relative to the negative control. These compounds are labeled with their IDs in the original library.

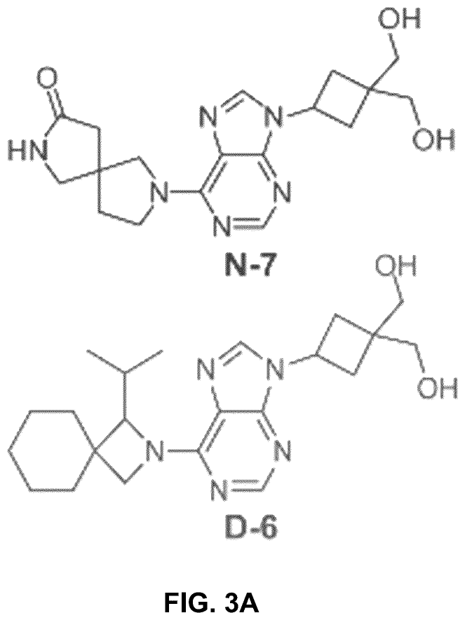

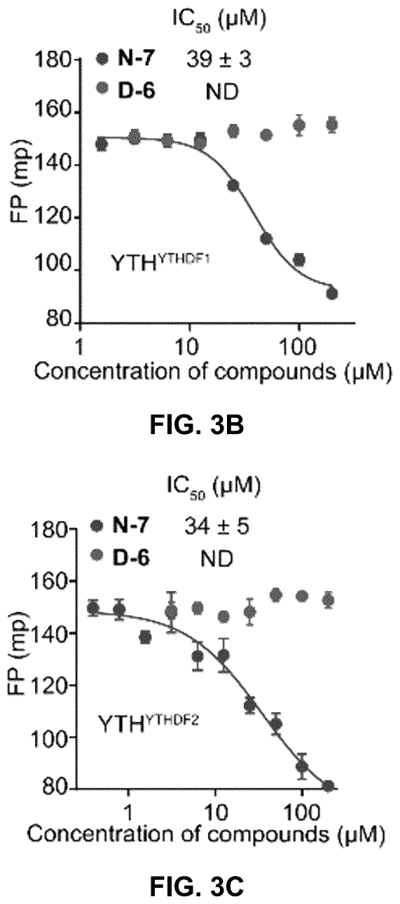

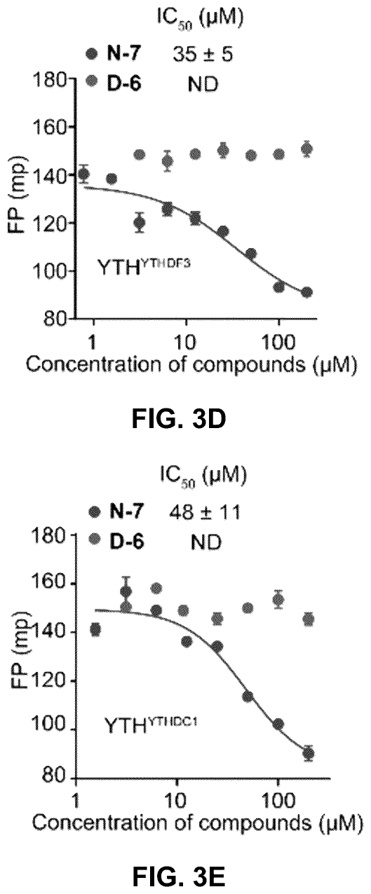

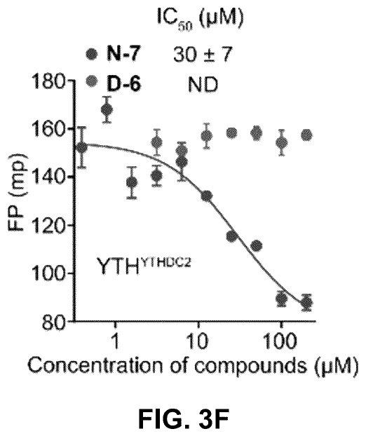

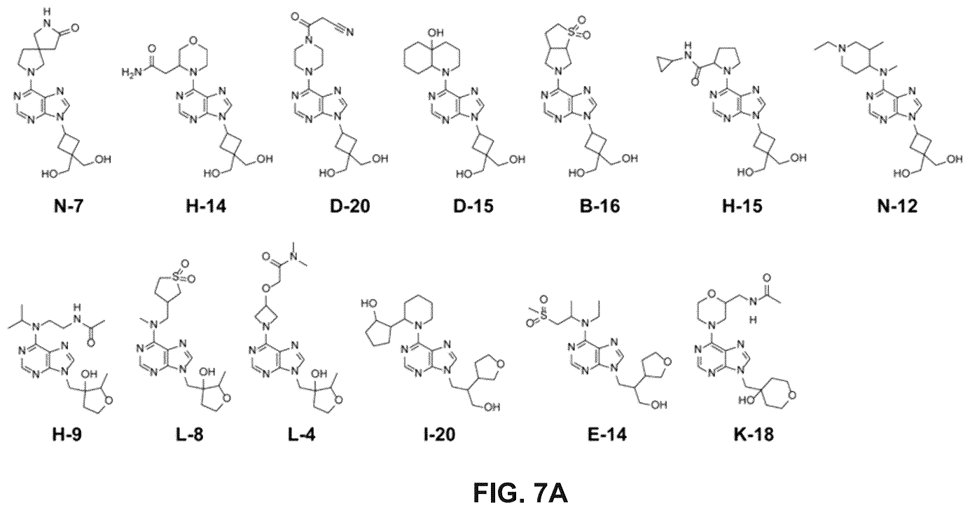

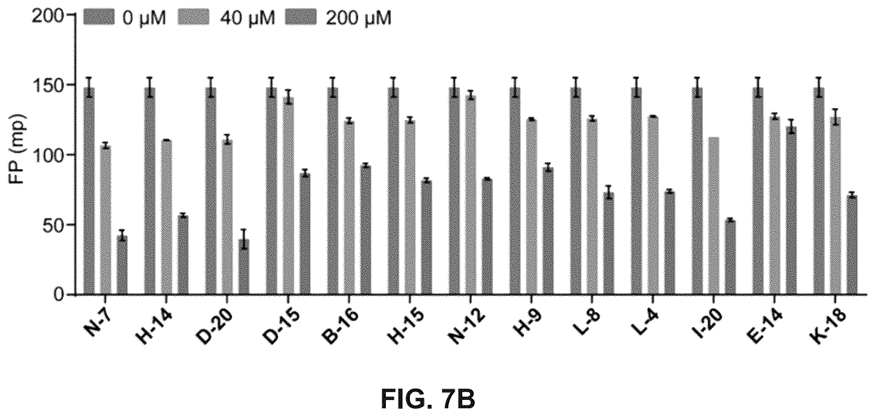

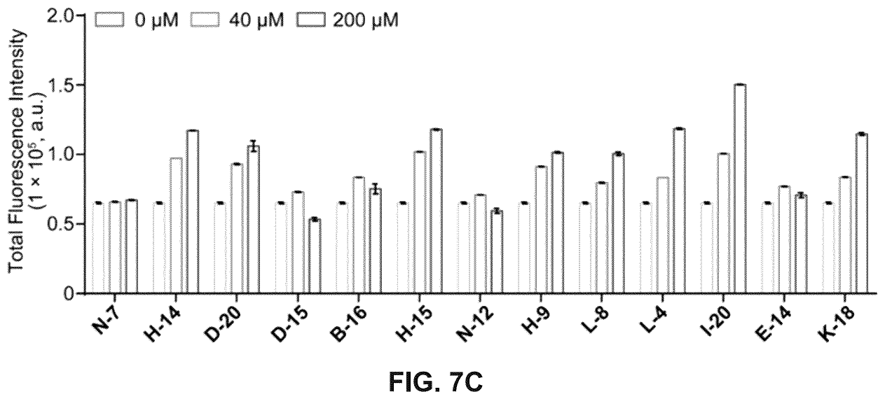

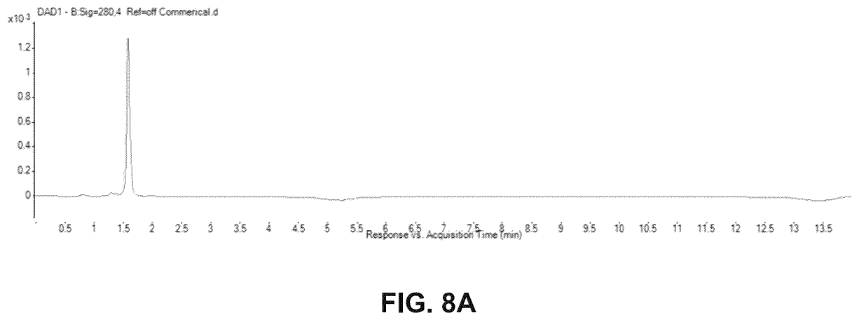

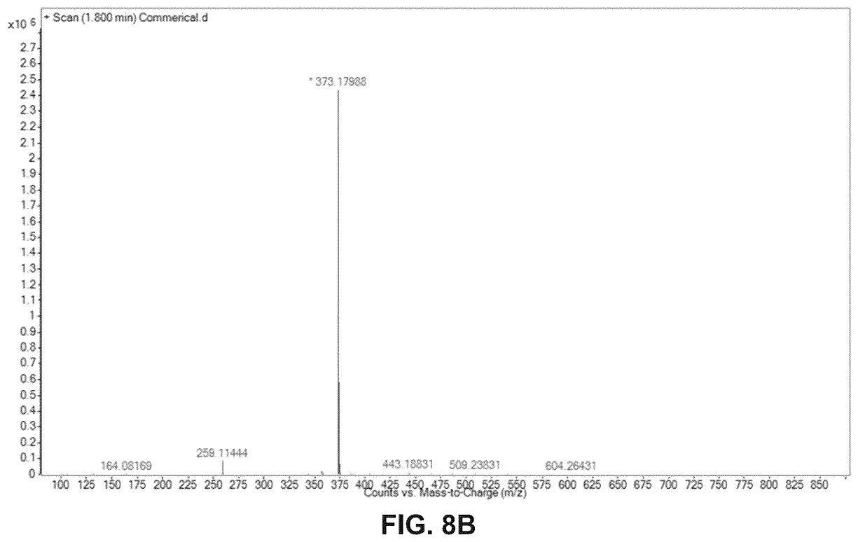

Interestingly, the chemical structures of all 13 compounds showed as analogues of adenosine with chemical modifications at the N9 and the N6 of adenine (see chemical structures in FIG. 7A). Moreover, 7 out of the 13 compounds (N-7, H-14, D-20, D-15, B-16, H-15, N-12) shared the same bis-hydroxymethyl cyclobutyl group at the N9 position (FIG. 7A), which increased our confidence in the relevance of the identified structures. We confirmed the reproducibility of the screening results by running the FP competition assay with higher compound dosages at 200 μM with replicates. The 13 compounds all showed a reduction of the FP signal in a dose-response manner (FIG. 7B). We chose the compound “N-7”, one of the compounds that showed the most significant inhibitory effect and did not interfere with the total fluorescence intensity of the assay, for further characterizations (FIG. 3A, FIG. 7B, and FIG. 7C, ESI). Meanwhile, we chose the compound “D-6” in the screened library as a negative control. D-6 shares the same purine and the [1-(hydroxymethyl)-cyclo-butyl]-methanol structures with N-7 but contains a 3-propan-2-yl-2-azaspiro-[3.5]-nonane group, rather than the 2,7-diazaspiro[4.4]nonan-8-one group in N-7 (see comparison of chemical structures in FIG. 3A). We performed dose-response curve measurement to quantify the inhibitory activity (i.e., IC₅₀) of N-7 via the FP competition assay. N-7 (FIG. 8A, ESI, FIG. 8B, FIG. 8C) shows an inhibitory effect of the YTH^{YTHDF1} and FAM-m⁶A-RNA binding with an IC₅₀=39±3 μM, whereas D-6 shows no detectable competition (FIG. 3B).

N-7 is a pan-YTH domain inhibitor. In some aspects, a pan-YTH domain inhibitor can be a small molecule that can inhibit the interaction between YTH domains and m6A-modified transcripts. YTH domains are part of the YTH family of proteins, which are known as m6A readers. These proteins recognize m6A-modified transcripts and play a role in regulating the cellular fates of mRNAs.

One example of a pan-YTH domain inhibitor is the novel N-7, which was discovered by screening a nucleoside analogue library against the YTH domain of the YTHDF1 protein. N-7 was shown to inhibit five YTH domains from human YTHDF1, YTHDF2, YTHDF3, YTHDC1, and YTHDC2 proteins.

YTH domain-containing proteins are associated with many types of cancer, including pancreatic, colorectal, liver, esophageal, gastric, prostate, and lung cancer. For example, YTHDF1 is associated with poor prognosis in colorectal cancer and liver cancer. YTHDF2 is associated with pancreatic cancer, where it promotes proliferation while inhibiting tumor cell migration and invasion.

FIG. 3A shows the chemical structures of N-7 and D-6. FIG. 3B shows dose-response curves of the FP competition assay using compound N-7 or D-6 against YTH^{YTHDF1} protein. FIG. 3C shows dose-response curves of the FP competition assay using compound N-7 or D-6 against YTH^{YTHDF2} protein. FIG. 3D shows dose-response curves of the FP competition assay using compound N-7 or D-6 against YTH^{YTHDF3} protein. FIG. 3E shows dose-response curves of the FP competition assay using compound N-7 or D-6 against YTH^{YTHDC1} protein. FIG. 3F shows dose-response curves of the FP competition assay using compound N-7 or D-6 against YTH^{YTHDC2} protein. Data are presented as mean±SD (n=3). “ND” denotes “not detected by FP competition assay”.

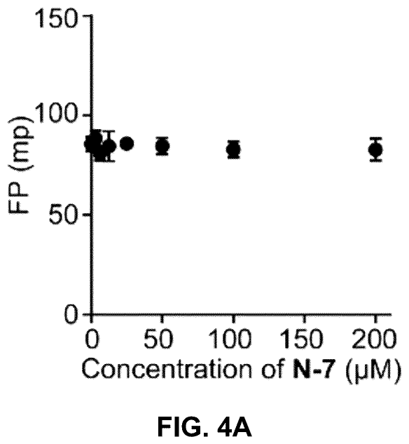

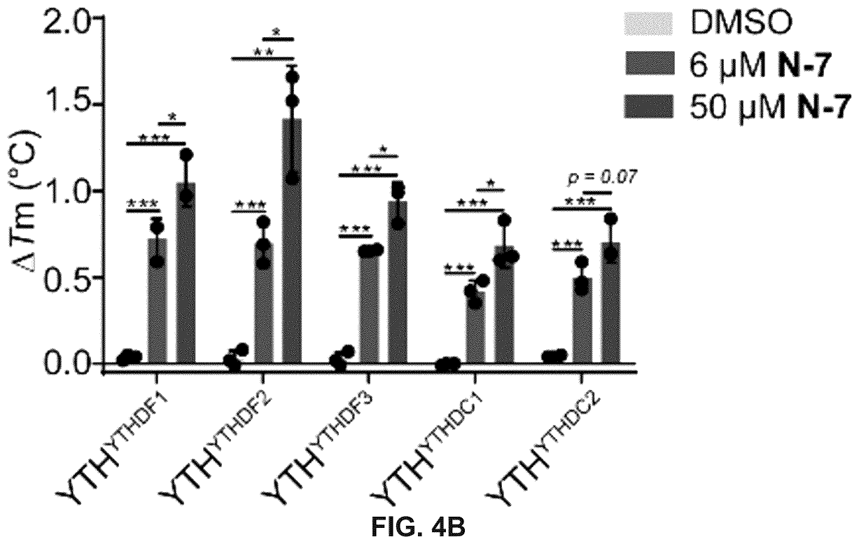

N-7 directly interacts with YTH-domain proteins. FIG. 4A shows the FP binding data between N-7 and the FAM-m⁶A-RNA. Data are presented as mean±SD with n=3 biological replicates and no binding was observed. FIG. 4B shows the results of the thermal shift assay performed on YTH^{YTHDF}1, YTH^{YTHDF2}, YTH^{YTHDF3}, YTH^{YTHDC1}, or YTH^{YTHDC2} proteins in the presence of compound N-7 with varying concentrations. Data are presented as mean±SD with n=3 biological replicates. The two-tailed t-test was used to assess the statistical significance of the difference between two samples, with the p-value indicated as “ns” for p≥0.05, “*” for p<0.05, “**” for p<0.01, or “***” for p<0.001. N-7 increases the mRNA expression level of PRR5 and CREBBP in HeLa cells.

Illustrations are provided for demonstrating the purifying and characterizing of YTH^{YTHDF1} protein. FIG. 5A shows the DNA sequence information of YTH^{YTHDF1}. FIG. 5B shows SDS-PAGE characterizations of the purified YTH^{YTHDF1} protein. YTH^{YTHDF1} protein was purified by a Nickel column with an imidazole concentration gradient in lysis buffer. FIG. 5C shows SDS-PAGE characterizations of the purity of YTH^{YTHDF1} protein before and after buffer exchange. This is the raw gel data for FIG. 1B. FIG. 5D shows full deconvolution of MS data of YTH^{YTHDF1} protein (black, observed mass; grey or red, theoretical mass) and zoom-in data are shown in FIG. 1B.

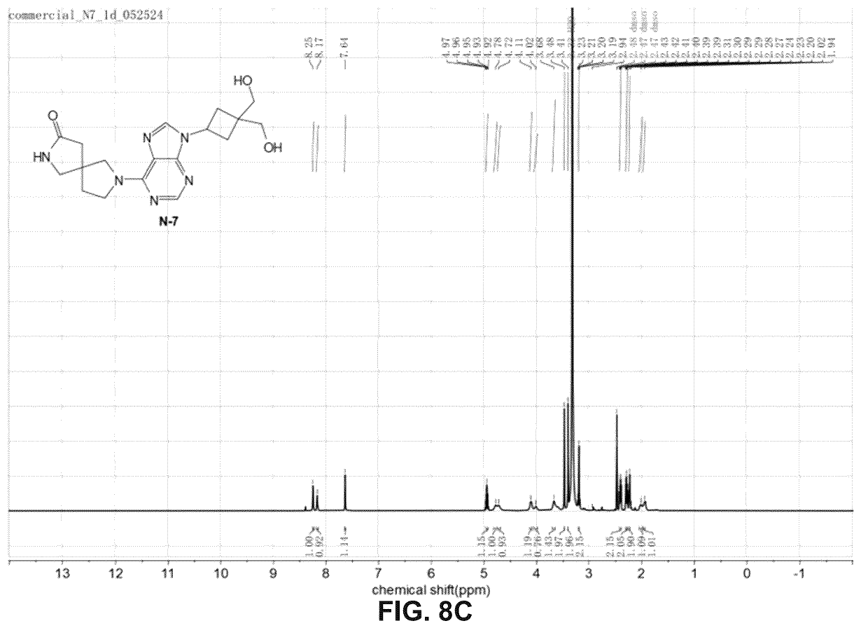

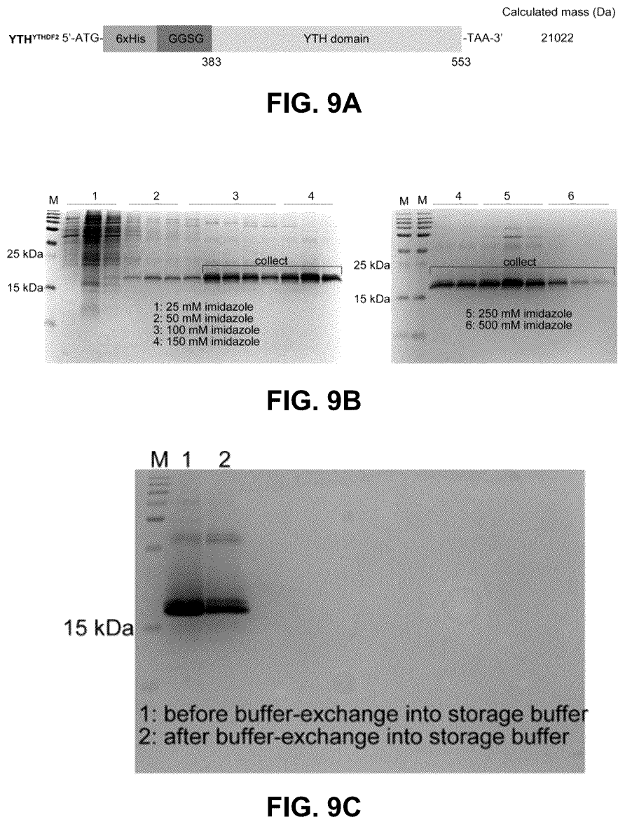

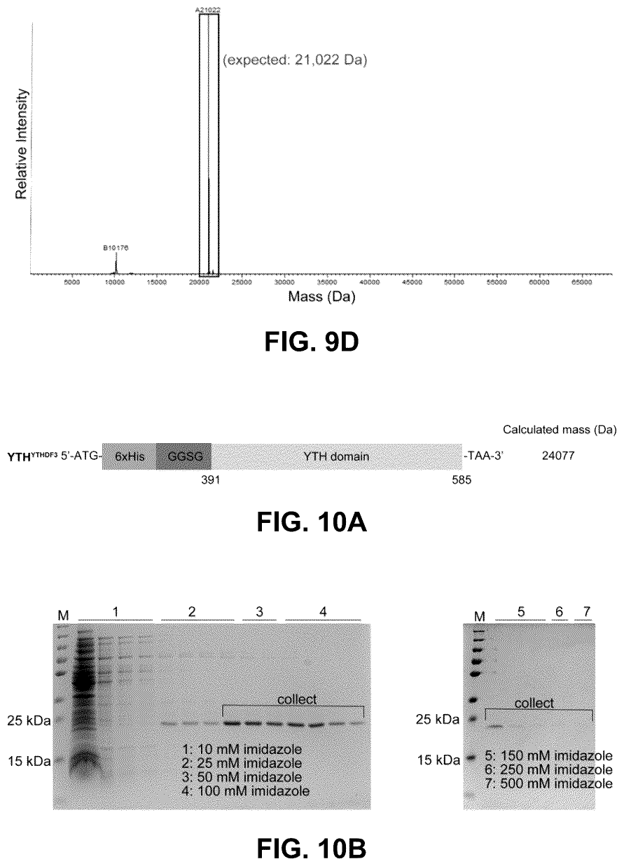

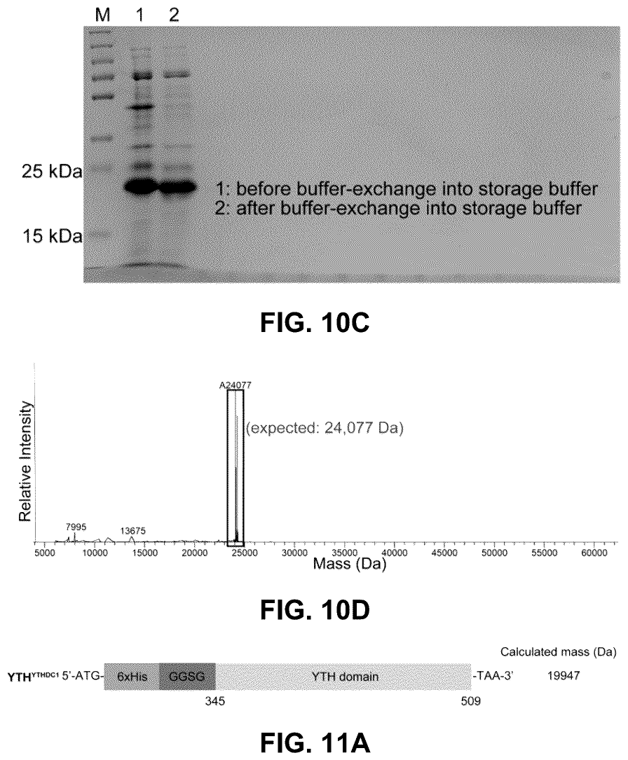

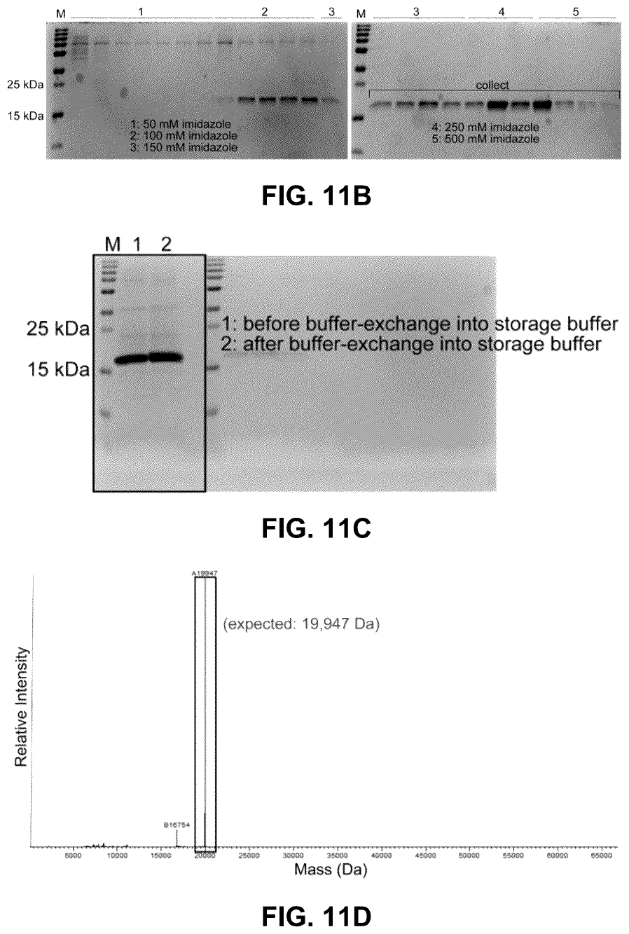

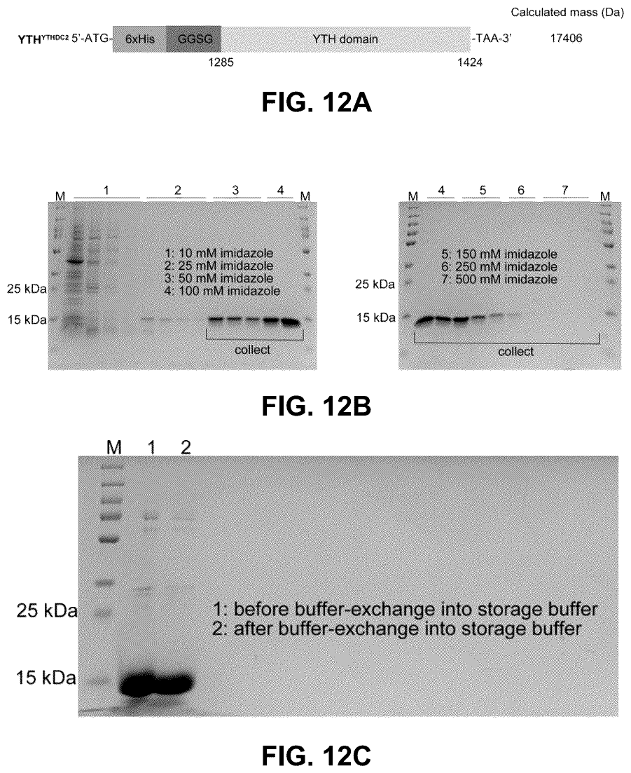

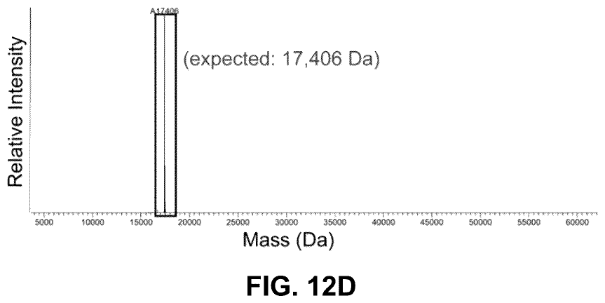

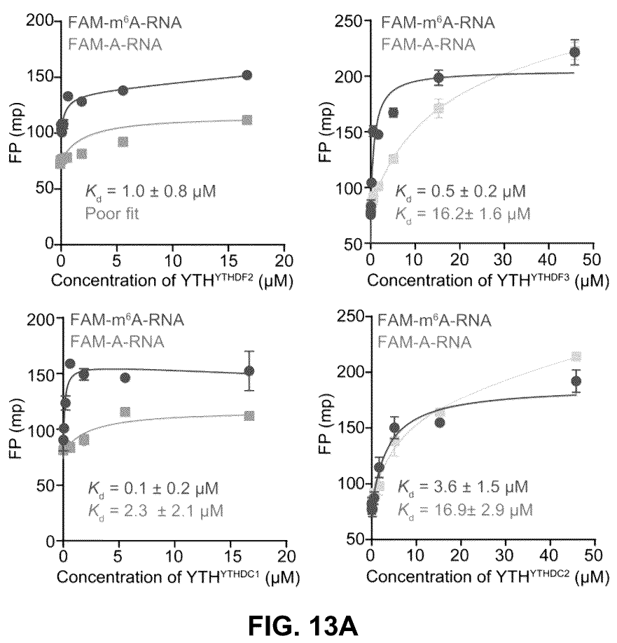

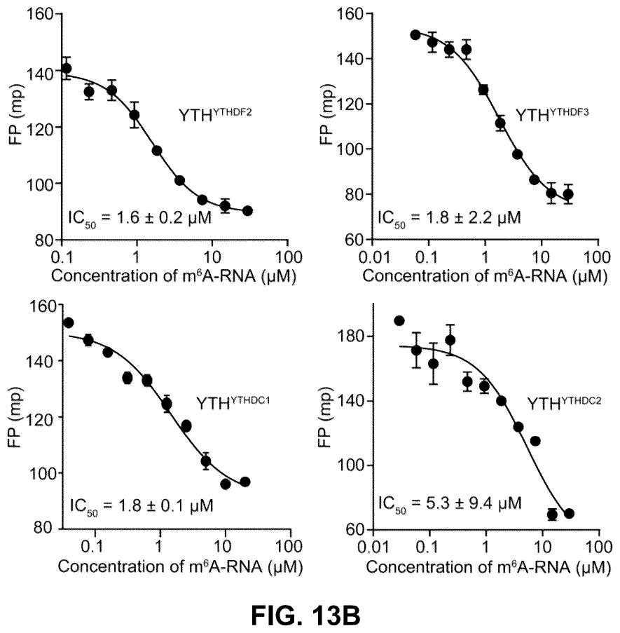

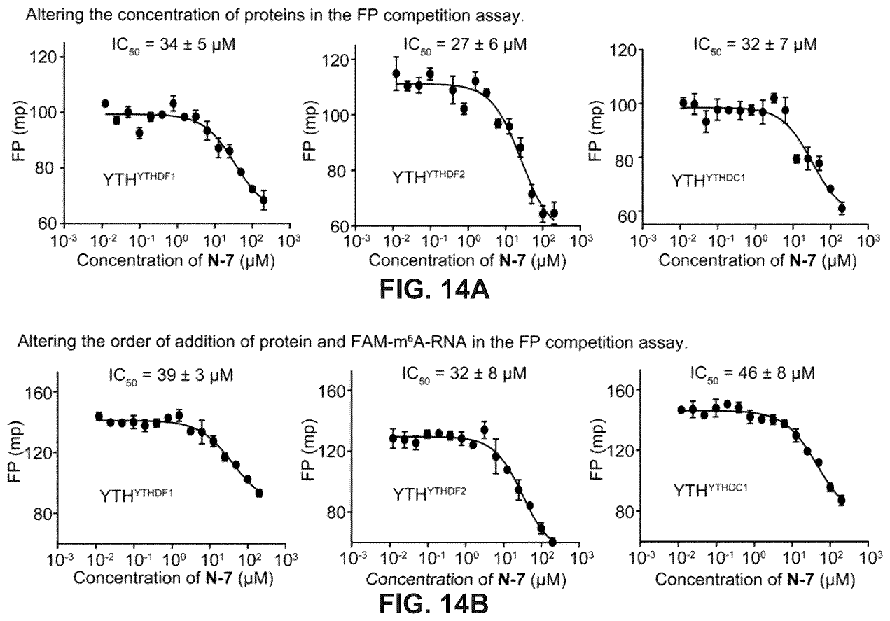

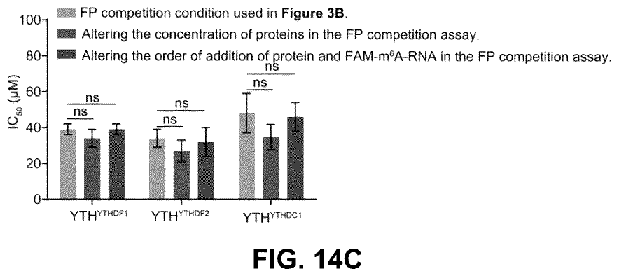

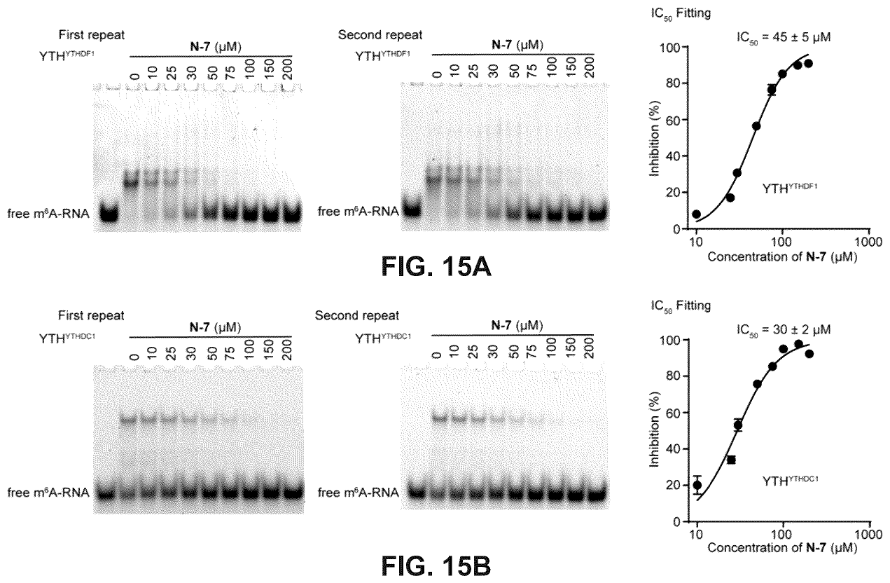

N-7 is apan-YTH-domain inhibitor: Next, we proceeded to examine the selectivity of N-7 against different YTH domains. We over-expressed and purified the YTH domains of human YTHDF2 (YTH^{YTHDF2}), YTHDF3 (YTH^{YTHDF3}), YTHDC1 (YTH^{YTHDC1}) and YTHDC2 (YTH^{YTHDC2}) proteins (FIGS. 9A-12D, ESI). First, we confirmed the purified YTH^{YTHDF2}, YTH^{YTHDF3}, YTH^{YTHDC1}, and YTH^{YTHDC2} as m⁶A readers in vitro by the FP binding assay. The measured binding affinities for all the YTH domains against the m⁶A-modified RNA agree with the reported values[11, 15, 16, 44], and were significantly higher relative to the binding with the unmodified RNA (FIG. 13A). We further benchmarked the FP competition assay for all purified YTH domain proteins with the non-fluorescent m⁶A-RNA as the positive control competitor. The results demonstrated that the non-fluorescent m⁶A-RNA competes effectively with the FAM-m⁶A-RNA in complex with each of the YTH proteins (FIG. 13B). We proceeded to test the inhibitory activity for N-7 against m⁶A-RNA bound with different YTH domains. Interestingly, N-7 exhibited comparable inhibitory activities against all purified YTH domains, with IC₅₀=39±3 μM for YTH^{YTHDF1}, 34±5 M for YTH^{YTHDF2}, 35±5 μM for YTH^{YTHDF3}, 48±11 μM for Y^{THYTHDC1}, and 30±7 μM for YTH^{YTHDC2} measured by the FP competition assay (FIG. 3B). The IC₅₀ values for N-7 against multiple YTH domains were robustly measured when altered protein concentration or the order of reagents addition was applied in the FP competition assay (FIG. 14A, FIG. 14B, FIG. 14C). In contrast, the negative control compound D-6 did not show competition against m⁶A RNA binding to all YTH-domain proteins (FIG. 3B). To further validate the measurements by the FP competition assay, we developed the RNA electrophoretic mobility shift assay (REMSA) [45] to assess inhibitory activity of small molecule inhibitors for YTH-m⁶A RNA binding. We measured the inhibitory activity of N-7 against YTH domains of YTHDF1 and YTHDC1 using REMSA, which yielded an ICsₒ=45±5 μM for YTH^{YTHDF1} and 30±2 μM for YTH^{YTHDC1} (FIG. 15A, FIG. 15B). The REMSA results agree with those measured by the FP competition assay within error (FIG. 3B). FIG. 3A shows the chemical structures of N-7 and D-6. FIG. 3B shows dose-response curves of the FP competition assay using compound N-7 or D-6 against YTHYTHDF1, YTHYTHDF2, YTHYTHDF3, YTHYTHDC1, and YTHYTHDC2 proteins. Data are presented as mean±SD (n=3). “ND” denotes “not detected by FP competition assay”.

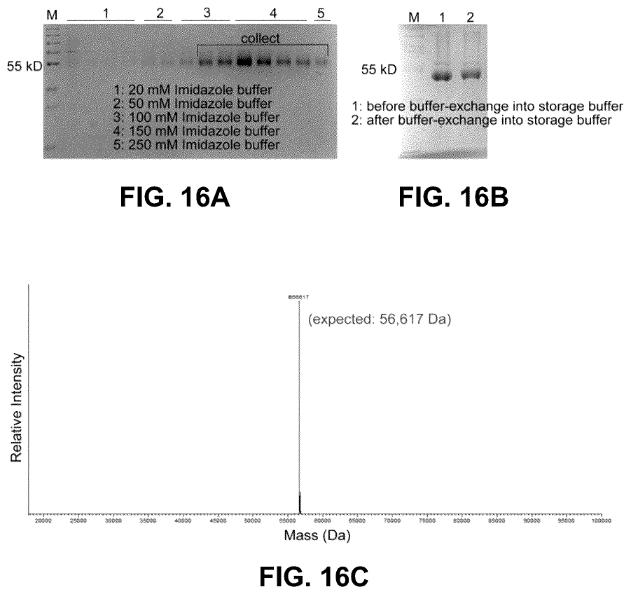

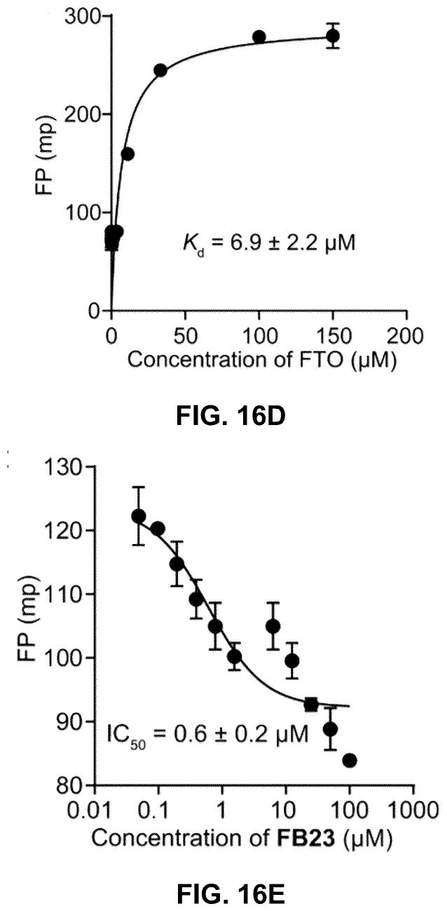

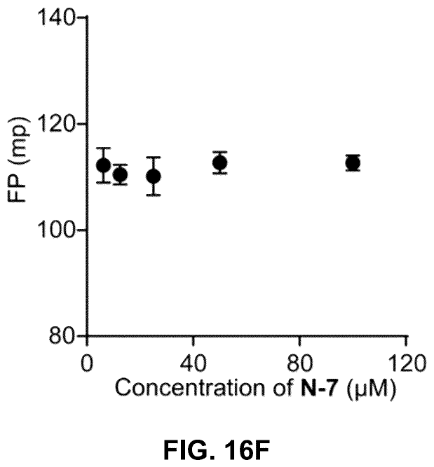

Given that N-7 is a pan-inhibitor for YTH domains, we investigate its inhibition selectivity against other m⁶A RNA recognition proteins that do not contain a YTH domain. We chose to test FTO, one of the eraser proteins that recognize m⁶A RNA and catalyze the removal of the methylation of m⁶A in the presence of co-factors Fe²⁺ and α-ketoglutarate[46]. We expressed and purified the FTO protein and confirmed binding between the purified FTO protein and FAM-m⁶A-RNA with the FP assay in the absence of catalytic co-factors (FIG. 16A, FIG. 16B, FIG. 16C, ESI, FIG. 16D). We benchmarked the FP competition assay by measuring the inhibitory activity of a reported FTO inhibitor FB23[47], which shows IC₅₀=0.6±0.2 μM (FIG. 16E). In contrast, N-7 did not show any detectable inhibition against FTO binding with m⁶A-RNA (FIG. 16F). This suggests that N-7 shows selective binding to YTH domain proteins over FTO.

Together, these findings show that N-7 is a pan-inhibitor for the recognition between YTH domains and m⁶A RNA, and presents selective inhibition against YTH domains over a non-YTH m⁶A recognition protein FTO.

N-7 Directly Interacts with YTH-Domain Proteins

To probe the inhibitory mechanism, we assessed interactions between N-7 and FAM-m⁶A-RNA or the YTH domain proteins separately. On the one hand, we observed no apparent binding between N-7 and FAM-m⁶A-RNA by directly titrating N-7 into a constant concentration of FAM-m⁶A-RNA via the FP binding assay (FIG. 4A).

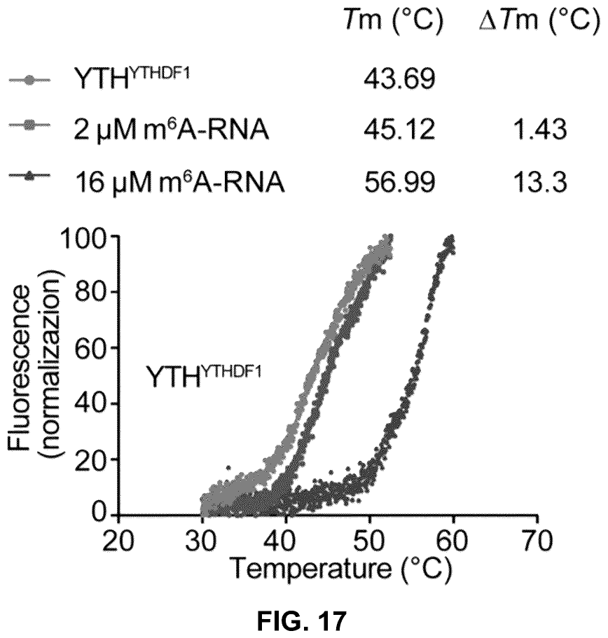

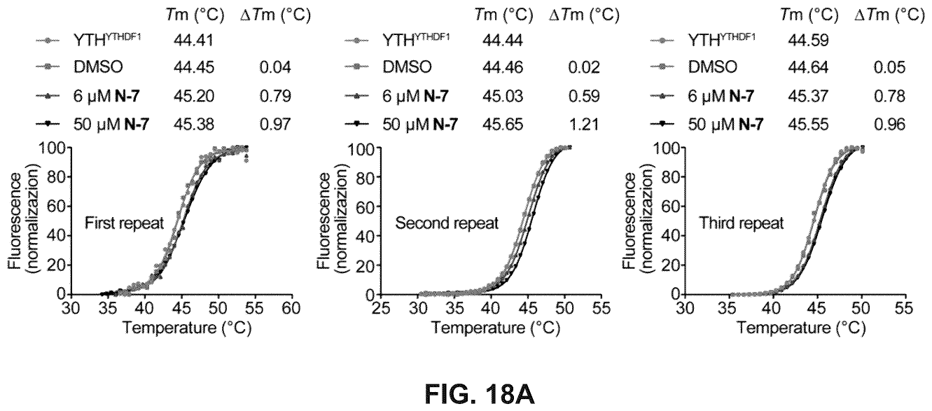

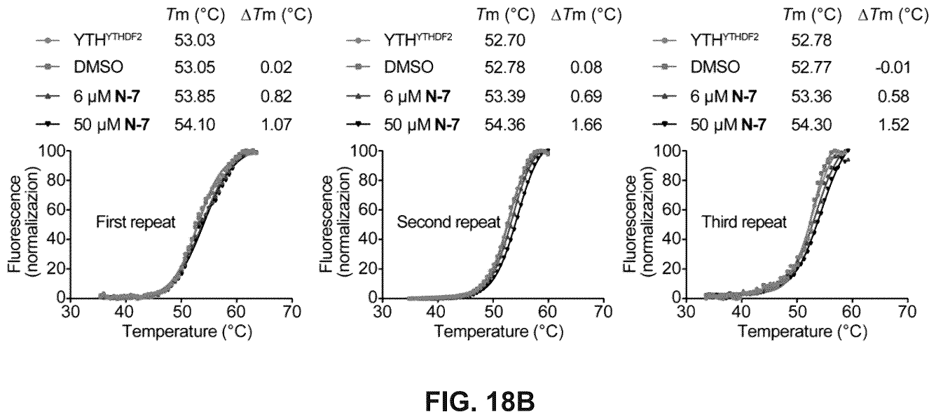

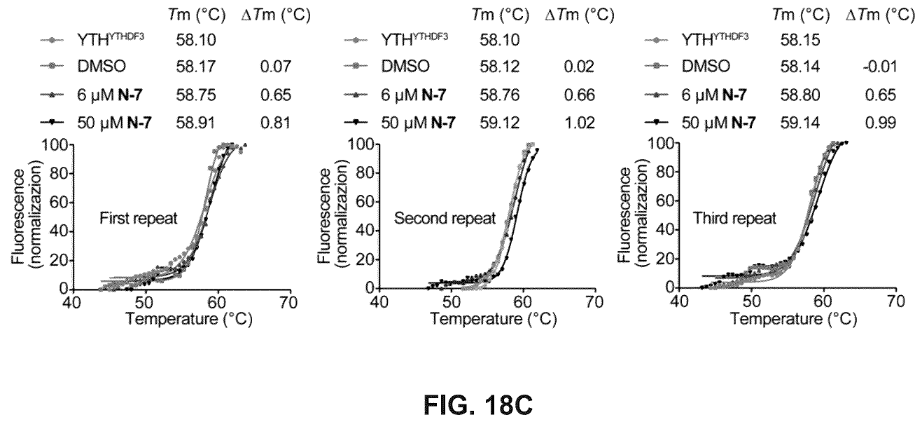

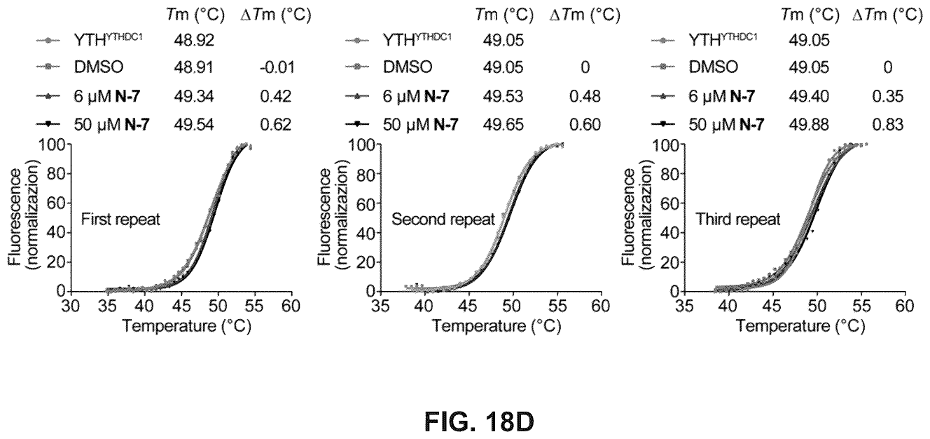

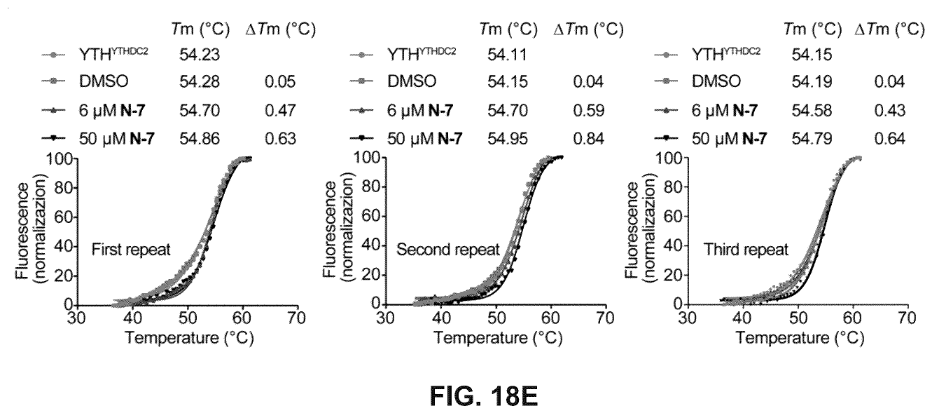

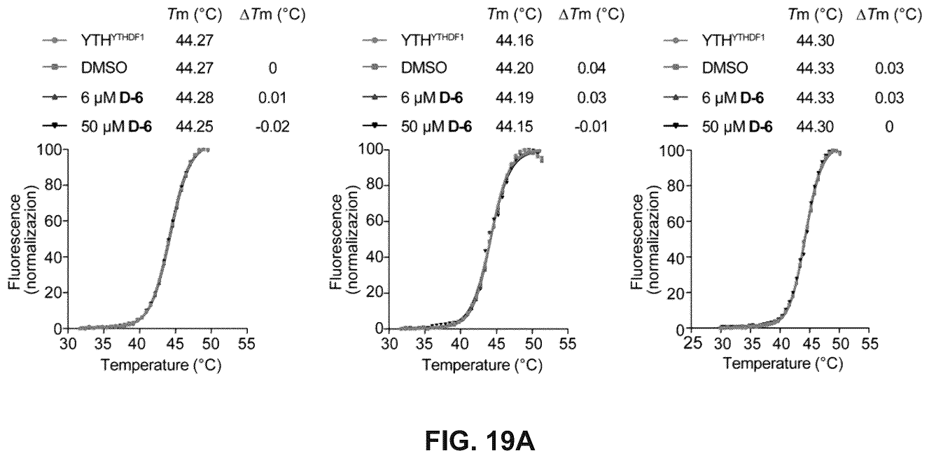

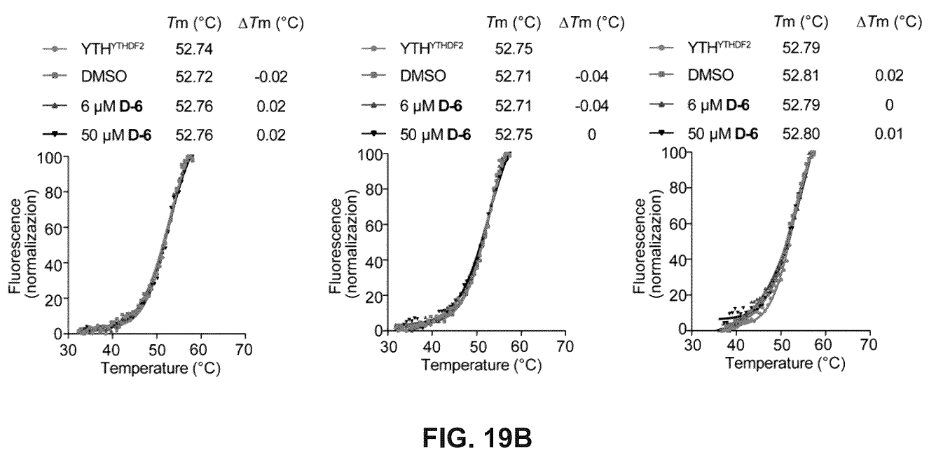

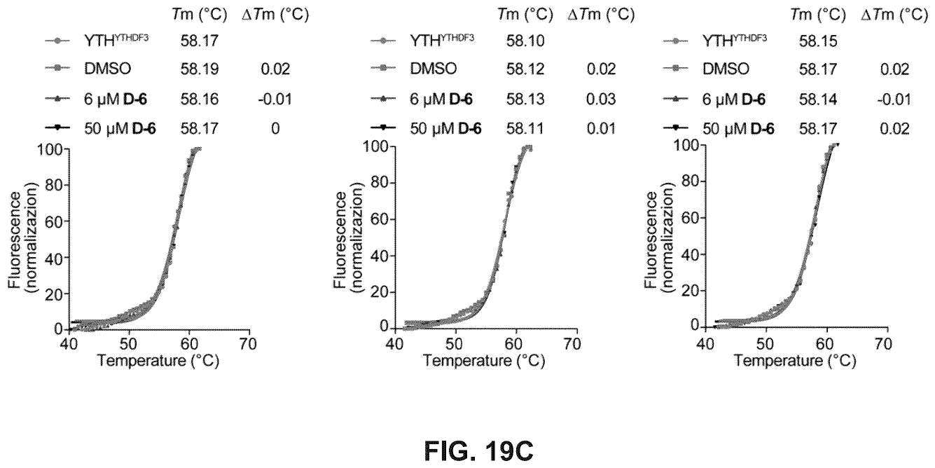

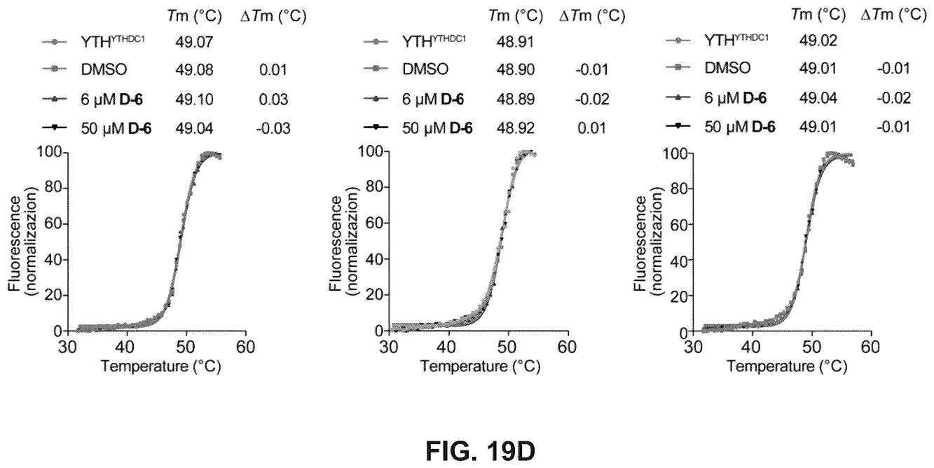

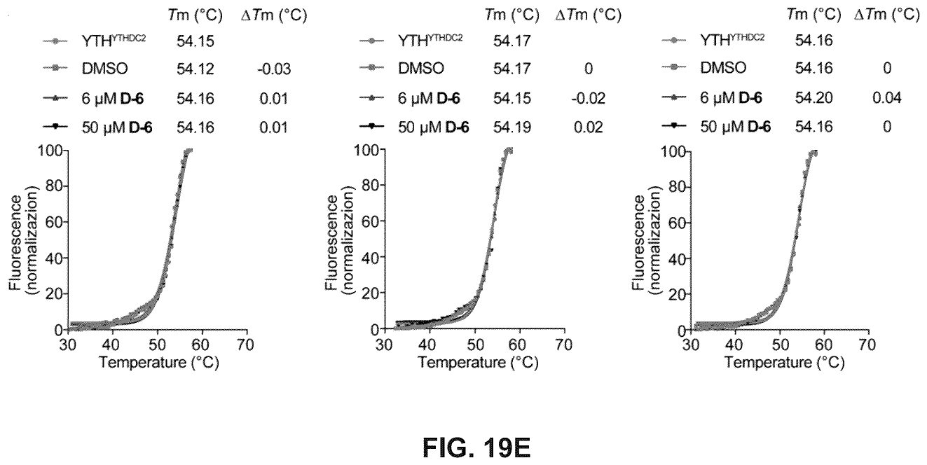

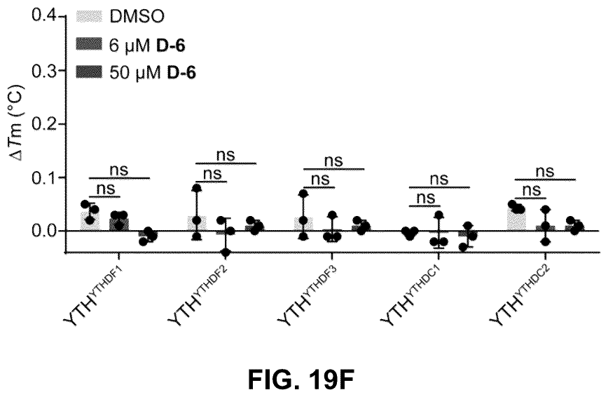

FIG. 4A shows the FP binding data between N-7 and the FAM-m6A-RNA. Data are presented as mean±SD with n=3 biological replicates and no binding was observed. FIG. 4B shows the results of the thermal shift assay performed on YTHYTHDF1, YTHYTHDF2, YTHYTHDF3, YTHYTHDC1, or YTHYTHDC2 proteins in the presence of compound N-7 with varying concentrations. Data are presented as mean±SD with n=3 biological replicates. The two-tailed t-test was used to assess the statistical significance of the difference between two samples, with the p-value indicated as “ns” for p≥0.05, “*” for p<0.05, “**” for p<0.01, or “***” for p<0.001. On the other hand, we investigated the interaction between N-7 and YTH domain proteins via the thermal shift assay[48]. As a positive control, we measured the thermal stabilization effect of the YTH^{YTHDF1} protein upon binding to m⁶A-RNA; incubation of 2 μM or 16 μM m⁶A-RNA significantly elevated the melting temperature of YTH^{YTHDF1} by 1.4° C. or 13.3° C., respectively (FIG. 17). Next, we characterized the effects of N-7 incubated with the five YTH domains. N-7 exhibited thermal stabilizations of all five YTH domain proteins; when 6 μM N-7 was incubated, the melting temperatures of the YTH domain proteins increased by 0.4-0.8° C. relative to the DMSO control (FIG. 4B and FIGS. 18A-18E). The stabilization effect became more prominent when an increasing amount of N-7 (50 μM) was added (FIG. 4B and FIGS. 18A-18E). In contrast, the negative control compound D-6 did not induce significant changes in the melting temperatures of the YTH domain proteins (FIGS. 19A-19F).

The thermal shift assay results suggested that N-7 inhibited the YTH and m⁶A RNA recognition likely through directly interacting with the YTH-domain proteins, rather than the RNA. The magnitude of melting temperature elevation caused by N-7 is significantly lower than that caused by the m⁶A-RNA binding; this is consistent with less favorable binding energetics between N-7 and the YTH domains compared to the modified RNA. We reason that this change in energetics can result from weakened hydrogen bonding, van der Waals, charge-charge interactions, and/or less favored hydrophobic effect for small molecule binding to the protein relative to the RNA[15].

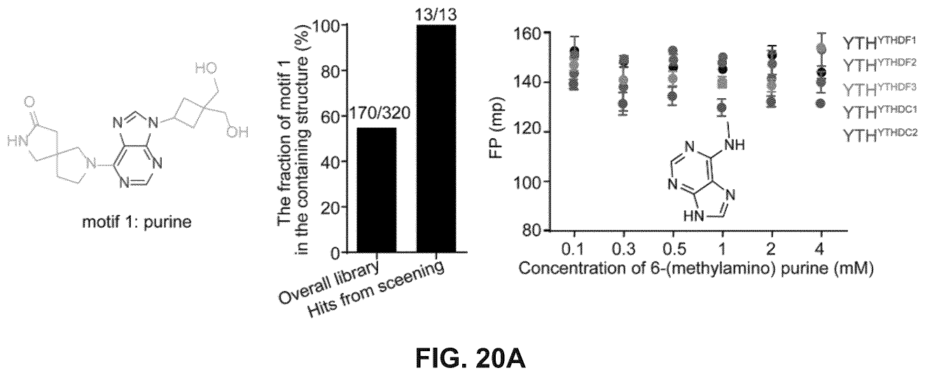

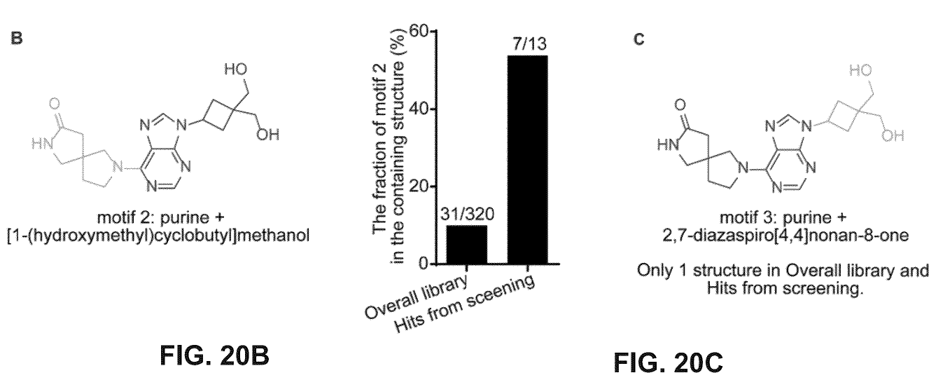

To gain further perspectives into potential structure-activity relationships (SAR), we performed an enrichment analysis of several structure motifs in N-7, regarding the occurrence frequency of each motif in the overall library (320 compounds) versus that in the identified hits (13 compounds shown in FIGS. 20A-20C, also see FIG. 7A). Motif 1 (i.e. the purine) appears in 53% of compounds in the overall library and 100% of the screening hits with ˜2-fold enrichment. This suggests that purine is an important fragment for the inhibitor structure, comparing to pyrimidine-derived structures within the library. Despite the importance of purine, we found no inhibitory activity for the 6-methylamino purine (FIG. 20A). This suggests the purine structure alone is not sufficient for presenting potency. Motif 2 (i.e. purine with the [1-(hydroxymethyl)cyclobutyl]methanol group) occurs in 10% of the overall library, of which the occurrence gets significantly enriched by >5-fold within the screening hits, where 54% (7 out of 13) of the hits compounds contain the Motif 2 structure. This suggests that Motif 2 can be critical for the binding with YTH domains. Only one compound (i.e. N-7) contains the Motif 3 (i.e. purine with 2,7-diazaspiro[4,4]nonan-8-one) in the overall library (FIG. 20C). It is difficult to assess any enrichment of this motif Among the 13 hit structures, Motif 3-equivalent positions showed greater variability than Motif 2, suggesting a certain tolerance of chemical diversity for the substitutes at the C6 of the purine (FIG. 20A).

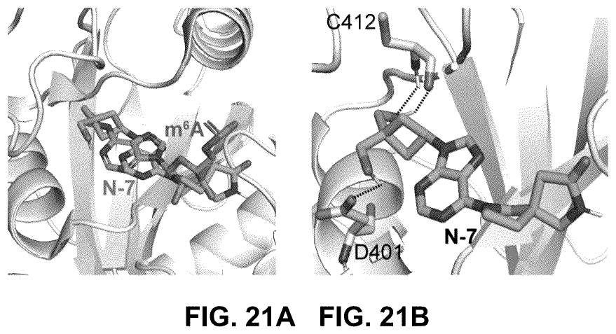

Lastly, we conducted molecular docking of N-7 onto the reported high-resolution structure of the YTH domain of YTHDF1 protein (PDBID: 4RCJ) to speculate the potential binding mode[15]. The best docking pose revealed that N-7 occupies the m⁶A binding pocket; the [1-(hydroxymethyl)cyclobutyl]methanol group of N-7 adopts the same orientation as the 6-methyl group of m⁶A (FIG. 21A, FIG. 21B). The docking result suggests potential polar contacts between two hydroxyl groups of N-7 with the backbone NH and C═O groups of C412, and the side chain of D401 (FIG. 21A, FIG. 21B). The suggested binding pose indicates the critical role of Motif 2 for the compound binding to the YTH domain. The Motif 3 orients outwards relative to the binding pocket, which is consistent with the chemical diversity we observed among the hits structures (FIG. 7A).

The motif enrichment analysis of the screening results and molecular docking suggested Motif 2 can be a critical motif for binding to the YTH domain, whereas the spiro group of N-7 can be more tolerating for diverse chemical groups. Future efforts in resolving the atomic-resolution structures of the N-7/YTH domain protein complexes and synthesizing N-7 fragments and analogue compounds are critical for providing insights into the mechanism of inhibition and elucidating the structure-activity relationship.

Comparison of N-7 with Reported YTH Domain Inhibitors

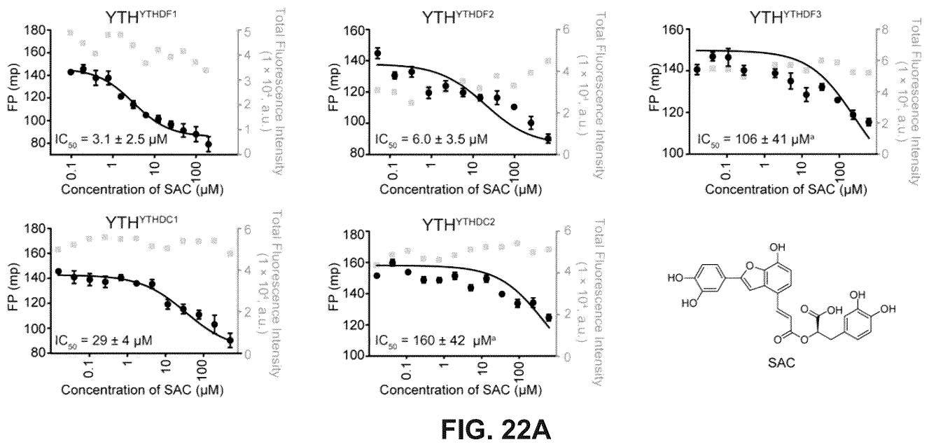

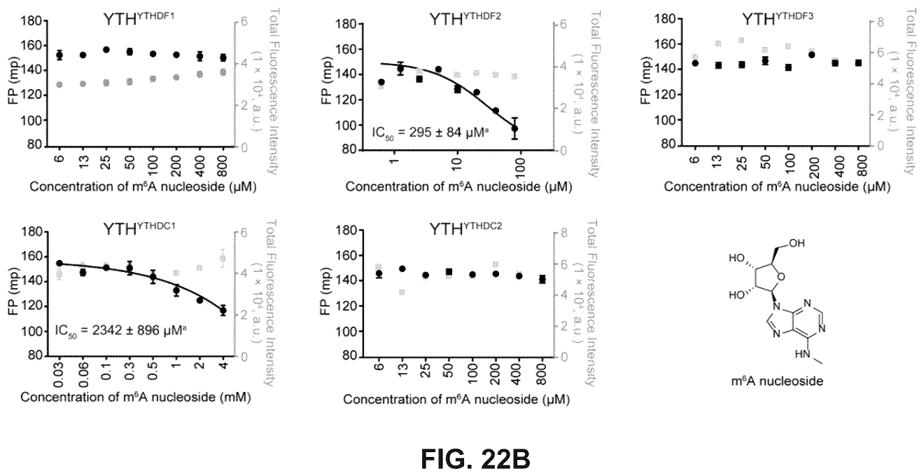

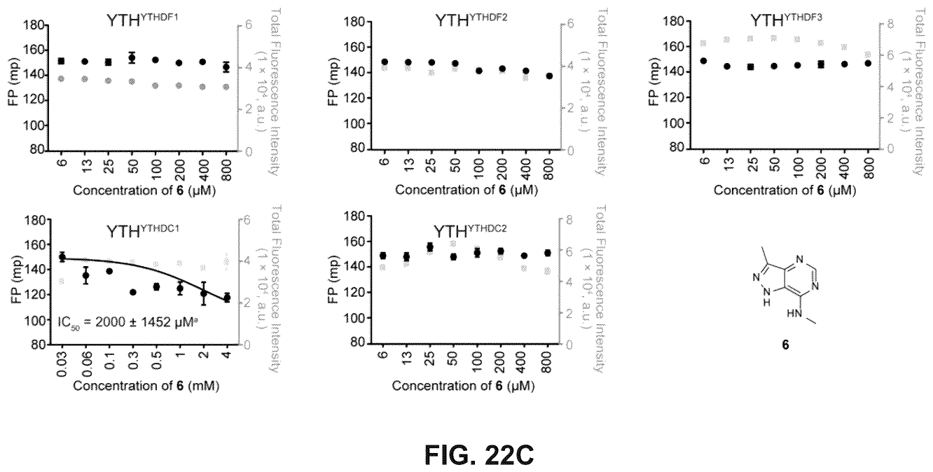

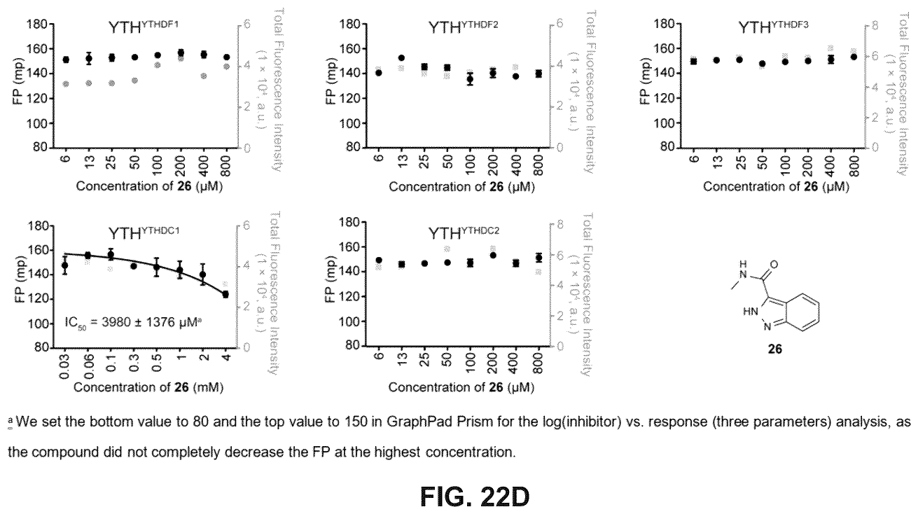

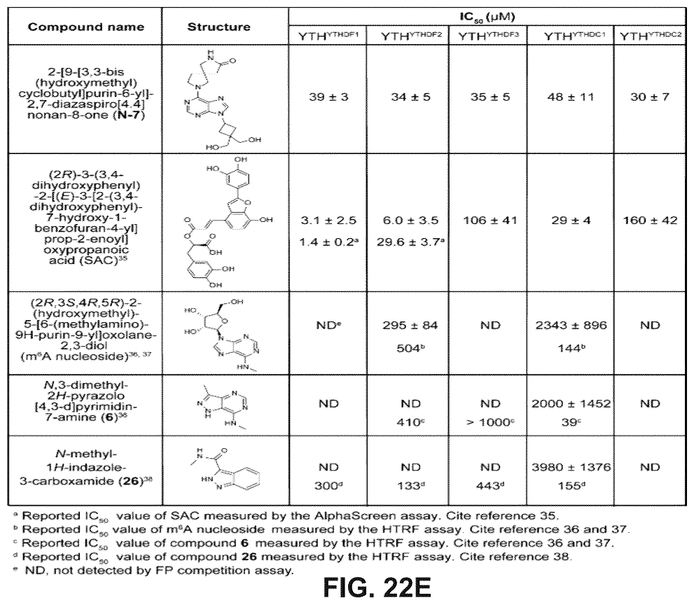

Next, we compared the inhibitory activity and selectivity of N-7 with four reported YTH domain inhibitors that are commercially available, including the natural product “SAC” [35], the “m⁶A nucleoside” [36, 37], a pyrazolopyrimidine derivative compound “6” [36], and an indazole derivative compound “26” [38]. These compounds were reported to inhibit YTH-m⁶A recognition against a single or subset of YTH domains in the literature, and inhibitory activities (i.e. IC₅₀) were measured by different assays including AlphaScreen[35] and the homogeneous time-resolved fluorescence (HTRF) assay[36-38]. As the IC₅₀ values are difficult to compare when measured by different assay conditions, we systematically measured the inhibitory activities of four reported inhibitors with the same FP competition assay against five YTH domains (see FIGS. 22A-22E). Interestingly, FIG. 22E shows a table with a Comparison of N-7 with reported YTH inhibitors based on the FP competition assay. Data are presented as mean±SD with n=2 or 3 biological replicates.

Based on our FP competition assay results, SAC exhibited inhibitory activity with an IC₅₀=3.1±2.5 μM against YTH^{YTHDF}1, consistent with the reported IC₅₀=1.4 0.2 M within error measured by the AlphaScreen Assay (FIG. 22E, FIG. 22A)[35]. Our data suggest a weak selectivity of SAC against YTH^{YTHDF2}, which showed two times higher IC₅₀ comparing to YTH^{YTHDF1}. Interestingly, we observed strong selectivity of SAC against YTH^{YTHDF3}, YTH^{YTHDC1}, and YTH^{YTHDC2} with IC₅₀ values ranging from 29 to 160 μM (FIG. 22E, FIG. 22A). Unlike N-7 being a pan-inhibitor, SAC presents selectivity among different YTH domains[35]. This immediately suggests different binding modes of SAC with YTH domains from those of N-7; high-resolution structural and biochemical characterizations would be critical to elaborate the differences in molecular recognition.

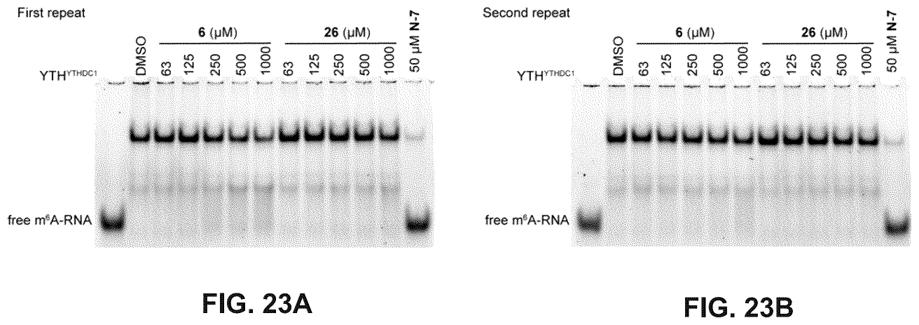

The m⁶A nucleoside, compound 6, and compound 26 were identified and validated as binding fragments to YTH^{YTHDF2} and YTH^{YTHDC1} by high-throughput X-ray crystallography to aid structure-based inhibitor design for YTH domain proteins[36-38]. These fragments show relatively weak inhibitory activities with the IC₅₀ values reported within the sub-millimolar to millimolar range measured by the HTRF assay[38](Table 1). Our measurements showed very weak inhibitory activities of the m⁶A nucleoside, 6 and 26 against all five YTH domains (IC₅₀>2 mM or not detectable), except that m⁶A nucleoside shows IC₅₀=295±84 μM against YTH^{YTHDF2} (FIG. 22B). Given the discrepancies of IC₅₀ values measured by the FP assay results and HTRF for compounds 6 and 26 against YTH^{YTHDC1}, we performed orthogonal competition REMSA to confirm the minimal inhibitory activity for compounds 6 and 26 (FIG. 23A, FIG. 23B). While these fragments (6 and 26) can fit into the binding pocket in the crystal structures[36, 38], the molecules alone do not appear to have high inhibitory activities, which suggests additional interactions are required to compete with m⁶A RNA binding.

In summary, among the compared compounds, SAC and N-7 are the most promising inhibitor structures. SAC contains more aromatic structures and presents a higher number of H-bond donors and acceptor groups than N-7, which can account for the selectivity among different YTH domains for SAC whereas N-7 shows as a pan-inhibitor. The N-7 structure may contain a robust motif that is important for binding to the highly similar pockets of all YTH domains[15]. At the current stage, both SAC and N-7 compounds showed in vitro inhibitory activity in the micromolar range and will benefit from further structure optimization efforts for improving potency.

In some embodiments, the techniques described herein relate to a method for inhibiting a pan-YTH domain, including: contacting the pan-YTH domain with a small molecule pan-YTH domain inhibitor, wherein the small molecule pan-YTH domain inhibitor is capable of inhibiting multiple YTH domains found in a YTH family of proteins that recognize m6A-modified transcripts, thereby changing a post-transcriptional modification process, wherein the small molecule pan-YTH domain inhibitor binds to a conserved region of the YTH domains and inhibits the recognition and binding of m6A-modified transcripts by the YTH family of proteins.

According to some aspects of the technology, the techniques described herein relate to a method, wherein the small molecule pan-YTH domain inhibitor is a chemical compound selected from the group consisting of small organic molecules, salts thereof, tautomers thereof, hydrates thereof, and/or solvates thereof, and wherein the chemical compound has a molecular weight of less than 1000 Daltons and is capable of crossing cell membranes.

In some embodiments, the techniques described herein relate to a method, wherein the small molecule pan-YTH domain inhibitor inhibits substantially all YTH domains in the YTH family of proteins by binding to the YTH domains with increased thermal stability of YTH domains upon inhibitor binding, and a selectivity of inhibition by at least 3-fold over non-YTH domain proteins.

According to some example aspects, the techniques described herein relate to a method, wherein the m6A-modified transcripts include N6-methyladenosine (m6A) modifications at consensus motifs.

In some embodiments, the techniques described herein relate to a method, wherein the post-transcriptional modification process is selected from the group consisting of RNA splicing, RNA stability, RNA localization, RNA translation, and RNA degradation, and wherein the alteration of the post-transcriptional modification process leads to changes in the abundance, half-life, subcellular localization, translation efficiency, or degradation rate of the m6A-modified transcripts.

According to some aspects of the technology, the techniques described herein relate to a method, wherein the YTH family of proteins includes YTHDF1, YTHDF2, YTHDF3, YTHDC1, and YTHDC2, and wherein each YTH family protein has a distinct function in the post-transcriptional regulation of m6A-modified transcripts, such as promoting translation (YTHDF1), promoting degradation (YTHDF2), promoting translation and degradation (YTHDF3), regulating splicing and nuclear export (YTHDC1), and regulating translation and stability (YTHDC2).

In some embodiments, the techniques described herein relate to a method, wherein the inhibition of the pan-YTH domain results in a decrease in the recognition and binding of m6A-modified transcripts by the YTH family of proteins, as measured by a reduction in the binding affinity of YTH family proteins to m6A-modified transcripts.

According to some aspects of the technology, the techniques described herein relate to a method, wherein the cell is a mammalian cell selected from the group consisting of human cells, mouse cells, rat cells, and non-human primate cells, and wherein the cell is a primary cell, a secondary cell, an immortalized cell line, a cancer cell, a stem cell, or an induced pluripotent stem cell.

In some embodiments, the techniques described herein relate to a method, wherein the organism is a mammal selected from the group consisting of humans, mice, rats, and non-human primates, and wherein the mammal is a model organism for studying the function of YTH family proteins or the role of m6A modification in disease.

According to some example aspects, the techniques described herein relate to a method, wherein the method is performed in vitro using cell-free assays, and wherein the assays are selected from the group consisting of binding assays, and high-throughput screening assays.

According to some aspects of the technology, the techniques described herein relate to a method, wherein the YTH family of proteins includes YTHDF1, YTHDF2, YTHDF3, YTHDC1, and YTHDC2, and wherein each YTH family protein has a distinct function in the post-transcriptional regulation of m6A-modified transcripts, such as promoting translation (YTHDF1), promoting degradation (YTHDF2), promoting translation and degradation (YTHDF3), regulating splicing and nuclear export (YTHDC1), and regulating translation and stability (YTHDC2).

In some embodiments, the techniques described herein relate to a method of treating a disease or disorder, including: contacting a pan-YTH domain in a human with a small molecule pan-YTH domain inhibitor, wherein the small molecule pan-YTH domain inhibitor is capable of inhibiting multiple YTH domains found in a YTH family of proteins that recognize m6A-modified transcripts, thereby changing a post-transcriptional modification process, wherein the small molecule pan-YTH domain inhibitor binds to a conserved region of the YTH domains and inhibits the recognition and binding of m6A-modified transcripts by the YTH family of proteins.

According to some aspects of the technology, the techniques described herein relate to a method for inhibiting a pan-YTH domain, including: contacting the pan-YTH domain with a pan-YTH domain inhibitor, wherein the pan-YTH domain inhibitor is a chemical that inhibits multiple YTH domains, thereby inhibiting the pan-YTH domain and changing a post-transcriptional modification process.

In some embodiments, the techniques described herein relate to a method, wherein the YTH domains are found in a YTH family of proteins that recognize m6A-modified transcripts, and wherein the YTH family of proteins includes YTHDF1, YTHDF2, YTHDF3, YTHDC1, and YTHDC2.

According to some example aspects, the techniques described herein relate to a method, wherein the pan-YTH domain inhibitor contacts and inhibits the multiple YTH domains by binding to a conserved pocket within the YTH domains that is involved in m6A recognition.

In some embodiments, the techniques described herein relate to a method, wherein the inhibition of the pan-YTH domain results in the changes in the post-transcriptional modification process, including alterations in mRNA stability, translation efficiency, and subcellular localization of m6A-modified transcripts.

According to some example aspects, the techniques described herein relate to a method, wherein the pan-YTH domain inhibitor is a small molecule with a molecular weight of less than 1000 Da.

In some embodiments, the techniques described herein relate to a method, wherein the YTH family of proteins contain the YTH domains that are evolutionarily conserved and structurally similar.

According to some example aspects, the techniques described herein relate to a method, wherein the YTH family of proteins identify the m6A-modified transcripts by recognizing the consensus m6A motif RRACH (R=G or A; H=A, C, or U) within the transcripts.

In some embodiments, the techniques described herein relate to a method, wherein the recognition of the m6A-modified transcripts by the YTH family of proteins results in the identification of the m6A-modified transcripts and their subsequent regulation by the YTH proteins.

According to some example aspects, the techniques described herein relate to a method, wherein the pan-YTH domain inhibitor is contacted with the pan-YTH domain at a concentration range of 1 nM to 1000 μM to inhibit the pan-YTH domain.

According to some example aspects, the techniques described herein relate to a method, wherein the pan-YTH domain inhibitor inhibits the pan-YTH domain to change the post-transcriptional modification process in a dose-dependent manner.

In some embodiments, the techniques described herein relate to a method, wherein the pan-YTH domain inhibitor is a chemical selected from the group consisting of small molecules, tautomers, hydrates, solvates, and/or salts thereof.

According to some example aspects, the techniques described herein relate to a method, wherein the chemical inhibits the multiple YTH domains with an IC50 value of 30-48 μM.

In some embodiments, the techniques described herein relate to a method, wherein the post-transcriptional modification process is changed by the inhibition of the pan-YTH domain, resulting in altered gene expression profiles and cellular phenotypes.

According to some example aspects, the techniques described herein relate to a method, wherein the pan-YTH domain is inhibited by contacting with the pan-YTH domain inhibitor in vitro using purified recombinant YTH proteins.

According to some example aspects, the techniques described herein relate to a method, wherein the pan-YTH domain inhibitor is a chemical that contacts and inhibits the multiple YTH domains by disrupting the interactions between the YTH domains and m6A-modified transcripts.

According to some example aspects, the techniques described herein relate to a method, wherein the pan-YTH domain inhibitor is a chemical that contacts the pan-YTH domain to inhibit the pan-YTH domain and change the post-transcriptional modification process, thereby providing a potential therapeutic approach for diseases associated with dysregulated m6A modification.

In some embodiments, the techniques described herein relate to a method for changing a post-transcriptional modification process, including: contacting a pan-YTH domain with a pan-YTH domain inhibitor, wherein the pan-YTH domain inhibitor is a chemical that inhibits multiple YTH domains, thereby inhibiting the pan-YTH domain and changing the post-transcriptional modification process.

According to some example aspects, the techniques described herein relate to a method for identifying pan-YTH domain inhibitors, the method including the steps of: (1) contacting an YTH domain of the YTHDF1 protein with a candidate inhibitor and/or FAM-labeled m6A RNA with a candidate inhibitor; and; and (2) executing FP HTS (fluorescence polarization, high-throughput screening) on the candidate inhibitor; whereby a potential inhibition provided by the candidate is indicated and a lead inhibitor is identified from a pool of candidate inhibitors.

In some embodiments, the techniques described herein relate to a method, wherein the YTH domain of the YTHDF1 protein is a recombinant protein expressed and purified from a bacterial cell expression system.

According to some example aspects, the techniques described herein relate to a method, wherein the candidate inhibitor is a small molecule compound selected from a library of diverse chemical structures, and wherein the library is a commercially available.

In some embodiments, the techniques described herein relate to a method, further including the step of determining the selectivity of the lead inhibitor by testing its ability to inhibit other YTH domain-containing proteins or unrelated RNA-binding proteins, and wherein the selectivity is assessed by comparing the IC50 values of the lead inhibitor against different protein targets.

In some embodiments, the techniques described herein relate to a method, wherein the FP HTS assay is performed in a high-throughput format using 384-well microplates to screen a large number of candidate inhibitors simultaneously.

In some embodiments, the techniques described herein relate to a method, wherein the lead inhibitor is used as a tool compound to study the biological functions of the YTHDF1 protein and the role of m6A modification in post-transcriptional gene regulation.

Conclusions: This current work reports the discovery of a new nucleoside analogue structure N-7 that functions as apan-inhibitor for YTH domain recognition of m⁶A-RNA and with IC₅₀=30-48 μM. N-7 exhibits a stabilizing effect of the YTH domains of human YTHDF1, YTHDF2, YTHDF3, YTHDC1, and YTHDC2 proteins by the thermal shift assay, and no direct interaction was observed between N-7 and RNA. This suggests the inhibitory mechanism primarily arose from the affinities between the compound and the YTH domain proteins. We provide that the development of new small molecule inhibitors will largely facilitate research into uncovering precise functions of m⁶A modification in physiology and diseases and potentiate future therapeutic efforts by targeting the epitranscriptome.

The technology enables a method for inhibiting a pan-YTH domain by contacting the domain with a small molecule pan-YTH domain inhibitor; wherein a pan-YTH domain inhibitor is a chemical that can inhibit multiple YTH domains, which are found in the YTH family of proteins that recognize m6A-modified transcripts; whereby the inhibition changes a post-transcriptional modification process.

The problem is, for example, that pan-YTH domains are implicated in a number of diseases including cancer and there are no small molecules that can effectively inhibit pan-YTH domains to treat diseases characterized by a post-transcriptional modification process.

The solution to help humans is a method for inhibiting a pan-YTH domain by contacting the domain with a small molecule pan-YTH domain inhibitor; wherein a pan-YTH domain inhibitor is a chemical that can inhibit multiple YTH domains, which are found in the YTH family of proteins that recognize m6A-modified transcripts; whereby the inhibition changes a post-transcriptional modification process.

In some examples, the novelty lies in the use of a small molecule inhibitor that targets multiple YTH domains, which are involved in recognizing m6A-modified transcripts, thereby altering post-transcriptional modification processes. In fact, the screening method used herein is novel and is discussed in more detail in the following Examples.

### EXAMPLES

### Example 1. Experimental Procedures

RNA oligonucleotides: RNA oligonucleotides including FAM-m⁶A-RNA, FAM-A-RNA, and m⁶A-RNA (FIG. 1C) were gifts from the He lab at the University of Chicago, which were originally in-house using an Expedite DNA synthesizer as previously reported[49]. We dissolved oligonucleotides into water (Fisher Science, BP561-1) for usage in vitro assays.

### Protein Expression and Purification

Genes of the YTH domain of YTHDF1 (amino acids 375-552), YTHDF2 (amino acids 383-553), YTHDF3 (amino acids 391-585), YTHDC1 (amino acids 345-509), and YTHDC2 (amino acids 1285-1424) were cloned into pET28a vector with an N-terminal His-tag for protein over-expressions in E. coli, respectively (FIGS. 5A-5D. and FIGS. 9A-121), ESI). We followed previously reported methods[15, 16, 44] to over-express and purify the YTH domain proteins from E. coli BL21(DE3) cells. A 5 mL overnight cell culture was used for inoculating 1 L of LB medium with 30 g/mL kanamycin. The cells were cultured at 37° C. with shaking at 250 r.p.m. until the OD₆₀₀ reached 0.6-0.8, which typically took around 3 hours. Subsequently, 0.5 mM Isopropyl β-D-1-thiogalactopyranoside (IPTG) was added into the cell culture to induce over-expression. Cells were then incubated in the shaker at 37° C. for 3 hours and then harvested by centrifugation at 2500 rpm for 20 minutes at 4° C.

We prepared the lysis buffers for different YTH domain proteins based on the published protocols: 20 mM HEPES, pH 7.5, 200 mM NaCl, and 1 mM dithiothreitol (DTT) for YTH^{YTHDF1} and YTH^{YTHDF2} proteins[50] and 100 mM Tris-HCl, pH 8.0, 500 mM NaCl and 1 mM DTT for YTH^{YTHDF3} protein[51] and 20 mM Tris-HCl, pH 7.5, 400 mM NaCl and 1 mM DTT for YTH^{YTHDC}1 and YTH^{YTHDC2} proteins[44, 52]. The cell pellets were resuspended and lysed in the corresponding lysis buffer by sonication for each protein. Clear cell lysates were obtained by taking the supernatant after centrifugation at 11,000 rpm for 15 minutes at 4° C. We then added 1% (w/v) streptomycin sulfate (Acros Organics) to the supernatant to precipitate genomic DNA, performed centrifugation at 11,000 rpm for 15 minutes at 4° C., and recovered the supernatant afterward. The collected supernatant was subsequently incubated with pre-equilibrated His60 Ni Superflow Resin (Takara Bio) at 4° C. for 1 hour and then loaded onto an empty column; resins (bound with protein) were washed extensively by >20 mL of 10 mM imidazole solutions in the lysis buffer, and proteins were eluted under a gradient of imidazole concentrations (25 mM, 50 mM, 100 mM, 150 mM, 250 mM, and 500 mM) in the lysis buffer, and fractions of eluted proteins were collected by 2 mL tubes. All collected fractions were examined through 12% SDS-PAGE and those that contained predominantly the targeted protein were pooled together (FIGS. 5A-5), and FIGS. 9A-12D, ESI), and concentrated by the concentrator (Sigma-Aldrich) down to around 3 mL. The concentrated protein was then desalted and exchanged into the storage buffer (25 mM HEPES, pH 7.5, 300 mM NaCl, 5% glycerol, 0.04% Triton X-100, and 1 mM DTT) using a PD-10 desalting column (GE Healthcare). The desalted proteins were concentrated to around 5 mg/mL, aliquoted, flash-frozen by liquid nitrogen, and stored at −80° C. Protein concentrations were measured via absorbance at 280 nm with 1 absorbance unit approximated to 1 mg/mL protein using a Nanodrop spectrophotometer (Thermo Scientific).

The FTO protein was overexpressed and purified from E. coli BL21 (DE3) cells following previously reported methods[53]. Briefly, one liter of cell culture was grown at 37° C. for 3 hours in LB medium with 30 g/mL kanamycin until the OD₆₀₀ reached 0.6-0.8. Overexpression was induced by 0.5 mM IPTG and cells were incubated at 18° C. for 14-16 hours. Cells were lysed in 40 mL lysis buffer (20 mM Tris-HCl, 300 mM NaCl) by sonication, in the presence of a protease inhibitor and further purified with His60 Ni Superflow Resin. Proteins were eluted with 20 mM to 250 mM imidazole gradient in the context of the lysis buffer followed by the desalting column and finally kept in a storage buffer composed of 50 mM Tris-HCl pH 7.5, 150 mM KCl, 5% glycerol, and 0.04% Triton-X-100 at −80° C. (FIGS. 16A-16F).

### MS Analysis of Intact Proteins

Purified proteins were analyzed using an Agilent 1260 Infinity HPLC coupled with the 6230 ESI time-of-flight (ESI-TOF) mass spectrometer (MS). The protein samples were subjected onto a C8 column (100×4.5 mm Phenomenex Aeris 3.6-μm Widepore XB-C8) and run through the column under an increasing gradient (5-95%) of non-polar buffer B in the aqueous buffer A (Buffer A: 95% water, 5% acetonitrile, 0.1% formic acid; Buffer B: 5% water, 95% acetonitrile, 0.1% formic acid). The eluted protein was injected into the MS and analyzed under the positive-ion mode. Total protein masses for the purified YTH domains and FTO were calculated through deconvolution using MagTran (Amgen).

### FP Binding Assay for Measuring Binding Affinities

Fluorescence polarization (FP) experiments were performed following the previously reported FP assay conditions for FAM-labeled RNA and YTH domains[50, 54]. Briefly, experiments were conducted in the binding buffer containing 25 mM Tris-HCl, pH 7.5, and 150 mM NaCl[50]. The same binding buffer was used in this study unless otherwise specified. RNA samples were diluted to 50 nM from the stock solutions by the binding buffer. YTH domain proteins were thawed, buffer exchanged into the binding buffer at 4° C. using the 10 kDa-cutoff concentrators (Sigma-Aldrich), and adjusted to 2× of the highest concentration used in the binding assay. 1 L RNase inhibitor (SUPERaseIn, Invitrogen) was added. 40 L protein solutions with various concentrations were prepared by serial dilution using the binding buffer. Subsequently, we added 40 L diluted RNA into each 40 L protein solution, mixed, and incubated at room temperature for 30 minutes. The final concentration of FAM-m⁶A-RNA or FAM-A-RNA was constant at 25 nM, and the YTH-domain protein concentrations varied from 10 nM to 24 μM. We performed three biological replicates for each binding measurement using the same protein purified from different batches. After incubation, samples were transferred to a black polypropylene 384-well round-bottom plate (Cellvis) and FP was measured on a Synergy™ Neo2 Multimode Microplate Reader at the wavelength of 485/20 nm for excitation and 528/20 nm for emission at 25° C.

We used the following equation to calculate the FP signal[43],

$$FP = \frac{{I}_{‖} - G \times {I}_{⊥}}{{I}_{‖} + G \times {I}_{⊥}}$$

where I_{∥} and I_{⊥} refer to the parallel or perpendicular polarized light intensity, respectively and we used 0.75 for the G-factor (G) calibrated based on the instrument guidance for the plate reader. The measured FP values at varying protein concentrations (x) and then fitted to the quadratic equation below describing single-site specific binding using the Levenberg-Marquardt non-linear curve fitting algorithm implemented in the Origin 2022 software:

$$FP = B + C \times ( D + {K}_{d} + x - \sqrt{{( D + {K}_{d} + x )}^{2} - 4 Dx} )$$

where D is the constant concentrations of FAM-m⁶A-RNA or FAM-A-RNA (D=25 nM), B and C relate with the FP for free RNA and bound RNA, and K_{d} is the dissociation constant.

Binding between FTO and FAM-m⁶A-RNA was performed as described above in the 50 mM borate binding buffer (pH 7.5).

To evaluate the binding affinity between FAM-m⁶A-RNA and N-7, we prepared eight N-7 compound solutions in the same binding buffer with 5% DMSO with 0 to 400 μM concentrations via serial dilution. 20 μL of 50 nM of FAM-m⁶A-RNA was added to L compound solution. The final concentration for the FAM-m⁶A-RNA was 25 nM and that for N-7 ranged between 0 to 200 μM. Samples were incubated and FP was measured and analyzed as described above.

### FP Competition Assays for Dose-Response Measurements and Screening

To set up the FP competition assay, we first used the non-fluorescent “m⁶A-RNA” (FIG. 1C) as a positive control inhibitor. We incubated 40 nM to 25 μM m⁶A-RNA with 2 μM YTH^{YTHDF1} and 25 nM FAM-m⁶A-RNA in the binding buffer with added RNase inhibitor at room temperature for 30 minutes. Samples were transferred onto the 384-well plate and measured on the plate reader as described above. We obtained the dose-response curves by plotting the FP values against concentrations of inhibitors. The inhibitory curves and IC₅₀ values were obtained using the “Dose-response Inhibition” model in GraphPad Prism6.0™. The same assay condition was used for benchmarking the competition assay for other YTH domains (FIG. 13A), except that 10 μM YTH^{YTHDC2} was used instead of 2 μM due to weaker binding affinity (or increased Kd) for YTH^{YTHDC2} to m⁶A-RNA compared to other YTH domains (FIG. 13A).

To assess the performance of the FP competition assay for screening, we performed the assay with positive and negative control inhibitors for 20 times each, where we used 20 μM non-fluorescent m⁶A-RNA and blank binding buffer as the positive and negative control, respectively. We calculated the Z′ factor using the following equation[43] based on the measured FP of the positive and negative controls,

$${Z}^{′} = 1 - \frac{3 \times ( SDpos + SDneg )}{\vert Mpos - Mneg \vert}$$

where SD and M represent the standard deviations and means of the 20 repeated measurements.

We purchased the Nucleoside Mimetics library from Enamine which contains 320 compounds dissolved in DMSO at 10 mM. All stock solutions of small molecules were prepared in 100% DMSO unless otherwise specified. To perform screening compounds, we first prepared RNA-protein solutions on the 384-well plate with 33.6 μL per well. We added 1.4 μL 1 mM compound into the RNA-protein solutions in each well, with the final concentrations of the YTH^{YTHDF1}, FAM-m⁶A-RNA, and each compound at 2 μM, 25 nM, and 40 μM, respectively. Each compound was screened in technical duplicates (FIG. 2). Meanwhile, DMSO and the non-fluorescent m⁶A-RNA were used as the negative and positive controls for the inhibitory activity on the same plate. The plate was incubated at room temperature for 30 minutes and subjected to the plate reader for FP measurements.

To perform dose-response measurements, we ordered 40 mg of N-7 (Z2760966931) from Enamine. We examined the quality of purchased N-7 through LC-MS and ¹H NMR spectroscopy (FIGS. 8A-8C, ESI). The dose-response FP competition assay was set up similar to that described for the non-fluorescent m⁶A-RNA. Instead of the m⁶A-RNA, 1.5-200 μM N-7 were incubated with 2 μM YTH domain proteins and 25 nM FAM-m⁶A-RNA in the binding buffer.

As IC₅₀ values may be subjected to changes under varying assay conditions, we performed several control conditions for the FP competition assay. Specifically, we adjusted the concentrations of the YTH domain proteins from 2 μM to the concentration around the K_{d} for each protein binding m⁶A-RNA (FIG. 1D and FIG. 14A). Secondly, we tested the reported FP competition protocol which first incubated the compound with YTH domains, followed by adding the FAM-m⁶A-RNA[35](FIG. 14B). Under these conditions, the measured IC₅₀ values showed no significant changes (FIG. 14C).

Reported inhibitors compound 6 and compound 26 were ordered from Chemspace (CSMB00010870727 and CSSS00000159915). The m⁶A nucleoside was ordered from AmBeed. The salvianolic acid C was ordered from MedChemExpress (HY-N0319). 6-(methylamino) purine was purchased from Fisher Scientific. The inhibitory activities of these compounds were examined using the same dose-response FP competition assay as N-7.

### RNA Electrophoretic Mobility Shift Assay (REMSA)

Competitive REMSA was performed by incubating 20 nM YTH^{YTHDF1} or YTH^{YTHDC1} with various concentrations of N-7 (1-200 μM) and 25 nM FAM-m⁶A-RNA in a buffer containing 10 mM HEPES, 50 mM KCl, 1 mM EDTA, 0.05% Triton-X-100, 5% glycerol and RNase inhibitor, in a final volume of 20 μL. The reaction mixture was incubated at room temperature for 20 minutes, and then loaded onto an 8% polyacrylamide gel containing 0.2% glycerol. Electrophoresis was performed in 0.5×TBE buffer at 90 V and 4° C. for 60 minutes. FAM-m⁶A-RNA was detected using the ChemiDoc™ MP imaging system (BIO-RAD) with the fluorescence blotting module.

### Thermal Shift Assay

Thermal shift assays were set up according to the reported assay conditions using the SYPRO orange dye, which exhibited enhanced fluorescence in a more hydrophobic environment typically during protein unfolding[48]. For the positive control, we incubated 2 M or 16 μM m⁶A-RNA with 4 μM YTH^{YTHDF1} in the binding buffer with added RNase inhibitor and 5×SYPRO orange dye (Invitrogen) at room temperature for 15 minutes. After incubation, the thermal shift assay was performed in the QuantStudio™ 3 Real-Time PCR System (Thermo Fisher). Each sample was heated from 25° C. to 75° C. ramping by 0.2° C./second; meanwhile, fluorescence intensities were recorded every 0.3° C. with the excitation and emission wavelengths at 470 nm and 570 nm, respectively. Data were first normalized by setting the highest and lowest fluorescence intensities in each melting run to 100% and 0%, respectively. Data were then fitted to obtain the melting temperatures (Tₘ) using the Boltzmann Sigmoid equation[48] within GraphPad Prism 6.0™. As the YTH^{YTHDF1} protein specifically bound with m⁶A-RNA with K_{d}=0.8±0.2 μM through the FP binding assay (FIG. 1D), we expected the presence of m⁶A-RNA would promote the thermal stability of the protein. As expected, we observed that the addition of m⁶A-RNA showed significant stabilization of the YTH^{YTHDF1} (FIG. 17).

To assess whether N-7 interacted with the YTH domain proteins, we used the thermal shift assay to examine if there were any stabilization effects of the YTH domains by adding N-7. We incubated 6 μM or 50 μM N-7 with 4 μM YTH^{YTHDF1}, YTH^{YTHDF2}, YTH^{YTHDF3}, YTH^{YTHDC1}, or YTH^{YTHDC2} in the same condition without the RNase inhibitor as above. As a negative control, we added an equal volume of DMSO rather than N-7 into each protein. The thermal shift assay measurements and analyses were performed in the same manner as described for the positive control. For each YTH domain protein, three biological replicates were performed to quantify the errors of the assay. The thermal stabilizations of the YTH domain proteins were analyzed by the change of the Tₘ (ΔTₘ) as the difference between the Tₘ measured on the protein with N-7 or DMSO, and the Tₘ of protein-only control (FIG. 4B and FIGS. 18A-18E). The interactions between D-6 and YTH domain proteins were assessed using the same method as N-7 (FIGS. 19A-19F).

Molecular Docking with AutoDock

The crystal structure of the YTHDF1 in complex with GGm⁶ACU (PDBID: 4RCJ) was chosen for molecular docking modeling[15]. The structure was prepared for docking by removing water molecules and the bound RNA oligonucleotide GGm⁶ACU. The structure of N-7 was prepared for docking by generating conformers and optimizing geometry by AutoDock4[55]. During molecular docking, the binding pocket of YTHDF1 was defined as 10 Å around the m⁶A binding site. The ligand N-7 was docked against the defined pocket of YTH^{YTHDF1} protein via AutoDock. The resulting binding pose of N-7 with the most favorable docking score was reported and analyzed.

### REFERENCES

1. E., P. R. P. K. D., Existence of methylated messenger RNA in mouse L cells. Cell, 1974. Volume 1: p. 37-42.
2. Meyer, K. D., et al., Comprehensive analysis of mRNA methylation reveals enrichment in 3′ UTRs and near stop codons. Cell, 2012. 149(7): p. 1635-46.
3. Dominissini, D., et al., Topology of the human and mouse m6A RNA methylomes revealed by m6A-seq. Nature, 2012. 485(7397): p. 201-6.
4. Desrosiers, R., K. Friderici, and F. Rottman, Identification of methylated nucleosides in messenger RNA from Novikoffhepatoma cells. Proc Natl Acad Sci USA, 1974. 71(10): p. 3971-5.
5. Liu, J., et al., N (6)-methyladenosine of chromosome-associated regulatory RNA regulates chromatin state and transcription. Science, 2020. 367(6477): p. 580-586.
6. Patil, D. P., et al., m(6)A RNA methylation promotes XIST-mediated transcriptional repression. Nature, 2016. 537(7620): p. 369-373.
7. Meyer, K. D., et al., 5′ UTR m(6)A Promotes Cap-Independent Translation. Cell, 2015. 163(4): p. 999-1010.
8. Wang, X., et al., N(6)-methyladenosine Modulates Messenger RNA Translation Efficiency. Cell, 2015. 161(6): p. 1388-99.
9. Shi, H., et al., YTHDF3facilitates translation and decay of N(6)-methyladenosine-modified RNA. Cell Res, 2017. 27(3): p. 315-328.
10. Roundtree, I. A., et al., YTHDC1 mediates nuclear export of N(6)-methyladenosine methylated mRNAs. Elife, 2017. 6.
11. Wang, X., et al., N6-methyladenosine-dependent regulation of messenger RNA stability. Nature, 2014. 505(7481): p. 117-20.
12. Liu, N., et al., N6-methyladenosine alters RNA structure to regulate binding of a low-complexity protein. Nucleic Acids Res, 2017. 45(10): p. 6051-6063.
13. Liu, N., et al., N(6)-methyladenosine-dependent RNA structural switches regulate RNA-protein interactions. Nature, 2015. 518(7540): p. 560-4.
14. Zaccara, S., R. J. Ries, and S. R. Jaffrey, Reading, writing and erasing mRNA methylation. Nat Rev Mol Cell Biol, 2019. 20(10): p. 608-624.
15. Xu, C., et al., Structural Basis for the Discriminative Recognition of N6-Methyladenosine RNA by the Human YT521-B Homology Domain Family of Proteins. J Biol Chem, 2015. 290(41): p. 24902-13.
16. Zhu, T., et al., Crystal structure of the YTH domain of YTHDF2 reveals mechanism for recognition of N6-methyladenosine. Cell Res, 2014. 24(12): p. 1493-6.
17. Flamand, M. N., M. Tegowski, and K. D. Meyer, The Proteins of mRNA Modification: Writers, Readers, and Erasers. Annu Rev Biochem, 2023. 92: p. 145-173.
18. Gao, Y., et al., Multivalent m(6)A motifs promote phase separation of YTHDF proteins. Cell Res, 2019. 29(9): p. 767-769.
19. Boo, S. H., et al., UPF1 promotes rapid degradation of m(6)A-containing RNAs. Cell Rep, 2022. 39(8): p. 110861.
20. Ries, R. J., et al., m(6)A enhances the phase separation potential of mRNA. Nature, 2019. 571(7765): p. 424-428.
21. Xiao, W., et al., Nuclear m(6)A Reader YTHDC1 Regulates mRNA Splicing. Mol Cell, 2016. 61(4): p. 507-519.
22. Park, O. H., et al., Endoribonucleolytic Cleavage of m(6)A-Containing RNAs by RNase P/MRP Complex. Mol Cell, 2019. 74(3): p. 494-507 e8.
23. Du, H., et al., YTHDF2 destabilizes m(6)A-containing RNA through direct recruitment of the CCR4-NOT deadenylase complex. Nat Commun, 2016. 7: p. 12626.
24. Wang, J., et al., Binding to m(6)A RNA promotes YTHDF2-mediated phase separation. Protein Cell, 2020. 11(4): p. 304-307.
25. Dai, X., et al., Identification of YTH Domain-Containing Proteins as the Readers for N1-Methyladenosine in RNA. Anal Chem, 2018. 90(11): p. 6380-6384.
26. Zheng, Q., et al., Cytoplasmic m(1)A reader YTHDF3 inhibits trophoblast invasion by downregulation of m(1)A-methylated IGF1R. Cell Discov, 2020. 6: p. 12.
27. Woodcock, C. B., et al., Biochemical and structural basis for YTH domain of human YTHDC1 binding to methylated adenine in DNA. Nucleic Acids Res, 2020. 48(18): p. 10329-10341.
28. Lence, T., et al., m(6)A modulates neuronal functions and sex determination in Drosophila. Nature, 2016. 540(7632): p. 242-247.
29. Shi, H., et al., m6A facilitates hippocampus-dependent learning and memory through YTHDF1. Nature, 2018. 563(7730): p. 249-253.
30. Liu, J., B. T. Harada, and C. He, Regulation of Gene Expression by N(6)-methyladenosine in Cancer. Trends Cell Biol, 2019. 29(6): p. 487-499.
31. Shi, R., et al., Linking the YTH domain to cancer: the importance of YTH family proteins in epigenetics. Cell Death & Disease, 2021. 12(4): p. 346.
32. Gokhale, N. S., et al., N6-Methyladenosine in Flaviviridae Viral RNA Genomes Regulates Infection. Cell Host Microbe, 2016. 20(5): p. 654-665.
33. Zhang, W., et al., Targeting m6A binding protein YTHDFs for cancer therapy. Bioorganic & Medicinal Chemistry, 2023. 90: p. 117373.
34. Liao, J., et al., Insight into the structure, physiological function, and role in cancer of m6A readers-YTH domain-containing proteins. Cell Death Discov, 2022. 8(1): p. 137.
35. Zou, Z., et al., FMRP phosphorylation modulates neuronal translation through YTHDF1. Molecular Cell, 2023. 83(23,7): p. 4304-4317.
36. Li, Y., et al., Structure-based design of ligands of the m6A-RNA reader YTHDC1. European Journal of Medicinal Chemistry Reports, 2022. 5: p. 100057.
37. Nai, F., et al., Fragment Ligands of the m6A-RNA Reader YTHDF2. ACS Medicinal Chemistry Letters, 2022. 13(9): p. 1500-1509.
38. Bedi, R. K., et al., Selectively Disrupting m(6)A-Dependent Protein-RNA Interactions with Fragments. ACS Chem Biol, 2020. 15(3): p. 618-625.
39. Micaelli, M., et al., Small-Molecule Ebselen Binds to YTHDF Proteins Interfering with the Recognition of N6-Methyladenosine-Modified RNAs. ACS Pharmacology & Translational Science, 2022. 5(10): p. 872-891.
40. Hong, Y.-G., et al., The RNA m6A Reader YTHDF1 Is Required for Acute Myeloid Leukemia Progression. Cancer Research, 2023. 83(6): p. 845-860.
41. Wang, L., et al., YTHDF2 inhibition potentiates radiotherapy antitumor efficacy. Cancer Cell, 2023. 41(7): p. 1294-1308.
42. Yang, S., et al., Discovery of a selective YTHDC1 inhibitor that targets acute myeloid leukemia. 2023, Research Square: Research Square
43. Moerke, N.J., Fluorescence Polarization (FP) Assays for Monitoring Peptide-Protein or Nucleic Acid-Protein Binding. Curr Protoc Chem Biol, 2009. 1(1): p. 1-15.
44. Xu, C., et al., Structural basis for selective binding of m6A RNA by the YTHDC1 YTH domain. Nat Chem Biol, 2014. 10(11): p. 927-9.
45. Dai, X., et al., Identification of YTH Domain-Containing Proteins as the Readers for N1-Methyladenosine in RNA. Analytical Chemistry, 2018. 90(11): p. 6380-6384.
46. Jia, G., et al., N6-methyladenosine in nuclear RNA is a major substrate of the obesity-associated FTO. Nat Chem Biol, 2011. 7(12): p. 885-7.
47. Huang, Y., et al., Small-Molecule Targeting of Oncogenic FTO Demethylase in Acute Myeloid Leukemia. Cancer Cell, 2019. 35(4): p. 677-691.e10.
48. Niesen, F. H., H. Berglund, and M. Vedadi, The use of differential scanning fluorimetry to detect ligand interactions that promote protein stability. Nat Protoc, 2007. 2(9): p. 2212-21.
49. Hu, L., et al., m6A RNA modifications are measured at single-base resolution across the mammalian transcriptome. Nature Biotechnology, 2022. 40(8): p. 1210-1219.
50. Liu, B., et al., A potentially abundant junctional RNA motif stabilized by m(6)A and Mg(2). Nat Commun, 2018. 9(1): p. 2761.
51. Li, Y., et al., Structural and Dynamic Insights into Redundant Function of YTHDF Proteins. Journal of Chemical Information and Modeling, 2020. 60(12): p. 5932-5935.
52. Ma, C., S. Liao, and Z. Zhu, Crystal structure of human YTHDC2 YTH domain. Biochem Biophys Res Commun, 2019. 518(4): p. 678-684.
53. Jia, G., et al., Oxidative demethylation of 3-methylthymine and 3-methyluracil in single-stranded DNA and RNA by mouse and human FTO. FEBS Lett, 2008. 582(23-24): p. 3313-9.
54. Zhou, H., et al., Characterizing Watson-Crick versus Hoogsteen Base Pairing in a DNA-Protein Complex Using Nuclear Magnetic Resonance and Site-Specifically (13)C-and (15)N-Labeled DNA. Biochemistry, 2019. 58(15): p. 1963-1974.
55. Morris, G. M., et al., AutoDock4 and AutoDockTools4: Automated docking with selective receptor flexibility. J Comput Chem, 2009. 30(16): p. 2785-91.
56. Huang, H., H. Weng, and J. Chen, m(6)A Modification in Coding and Non-coding RNAs: Roles and Therapeutic Implications in Cancer. Cancer Cell, 2020. 37(3): p. 270-288.
57. Roundtree, I. A., et al., Dynamic RNA Modifications in Gene Expression Regulation. Cell, 2017. 169(7): p. 1187-1200.
58. Fu, Y., et al., Gene expression regulation mediated through reversible m(6)A RNA methylation. Nat Rev Genet, 2014. 15(5): p. 293-306.
59. Liu, J., et al., A METTL3-METTL14 complex mediates mammalian nuclear RNA N6-adenosine methylation. Nat Chem Biol, 2014. 10(2): p. 93-5.
60. Bokar, J. A., et al., Purification and cDNA cloning of the AdoMet-binding subunit of the human mRNA (N6-adenosine)-methyltransferase. RNA, 1997. 3(11): p. 1233-47.
61. Zheng, G., et al., ALKBH5 is a mammalian RNA demethylase that impacts RNA metabolism and mouse fertility. Mol Cell, 2013. 49(1): p. 18-29.
62. Deng, X., et al., RNA N(6)-methyladenosine modification in cancers: current status and perspectives. Cell Res, 2018. 28(5): p. 507-517.
63. Zaccara, S. and S. R. Jaffrey, A Unified Model for the Function of YTHDF Proteins in Regulating m(6)A-Modified mRNA. Cell, 2020. 181(7): p. 1582-1595 e18.
64. Hsu, P. J., et al., Ythdc2 is an N(6)-methyladenosine binding protein that regulates mammalian spermatogenesis. Cell Res, 2017. 27(9): p. 1115-1127.
65. Han, D., et al., Anti-tumour immunity controlled through mRNA m(6)A methylation and YTHDF1 in dendritic cells. Nature, 2019. 566(7743): p. 270-274.
66. Einstein, J. M., et al., Inhibition of YTHDF2 triggers proteotoxic cell death in MYC-driven breast cancer. Mol Cell, 2021. 81(15): p. 3048-3064 e9.
67. Chang, G., et al., YTHDF3 Induces the Translation of m(6)A-Enriched Gene Transcripts to Promote Breast Cancer Brain Metastasis. Cancer Cell, 2020. 38(6): p. 857-871 e7.
68. Sheng, Y., et al., A critical role of nuclear m6A reader YTHDC1 in leukemogenesis by regulating MCM complex-mediated DNA replication. Blood, 2021. 138(26): p. 2838-2852.
69. Tanabe, A., et al., YTHDC2 Promotes Malignant Phenotypes of Breast Cancer Cells. J Oncol, 2022.2022:p. 9188920.
70. Li, Y., et al., Structural and Dynamic Insights into Redundant Function of YTHDF Proteins. J Chem Inf Model, 2020. 60(12): p. 5932-5935.
71. Micaelli, M., et al., Small-Molecule Ebselen Binds to YTHDF Proteins Interfering with the Recognition of N (6)-Methyladenosine-Modified RNAs. ACS Pharmacol Transl Sci, 2022. 5(10): p. 872-891.
72. Nai, F., et al., Fragment Ligands of the m(6)A-RNA Reader YTHDF2. ACS Med Chem Lett, 2022. 13(9): p. 1500-1509.
73. Hong, Y. G., et al., The RNA m6A Reader YTHDF1 Is Required for Acute Myeloid Leukemia Progression. Cancer Res, 2023. 83(6): p. 845-860.
74. Wang, L., et al., YTHDF2 inhibition potentiates radiotherapy antitumor efficacy. Cancer Cell, 2023. 41(7): p. 1294-1308 e8.
75. Yankova, E., et al., Small-molecule inhibition of METTL3 as a strategy against myeloid leukaemia. Nature, 2021. 593(7860): p. 597-601.

We claim:

1. A method for inhibiting a pan-YTH domain by contacting the domain with a small molecule inhibitor comprising Formula 1: 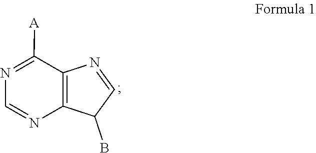
and/or a pharmaceutically acceptable salt thereof, hydrate and/or solvate thereof, tautomer thereof, and/or a prodrug thereof of Formula 1;
wherein each independent occurrence of A and/or B is independently selected from and/or comprises one or more of the following substituents and/or a salt, tautomer, hydrate and/or solvate thereof: 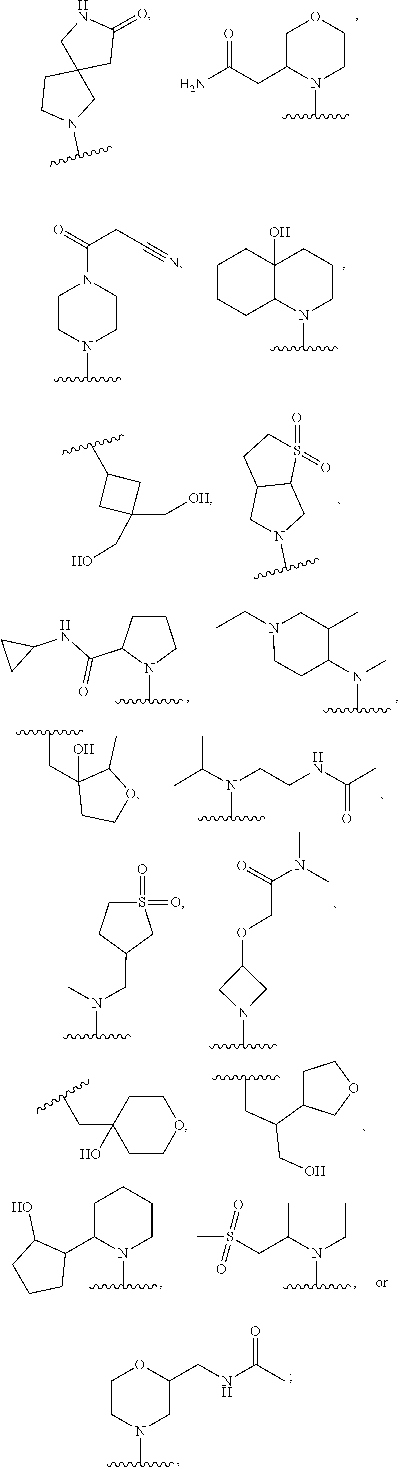
wherein each independent occurrence of [symbol] is a linker to Formula 1 and represents an attachment to an atom as indicated in Formula 1 and includes or is a combination of a single bond (−), a bond to a nitrogen, a bond to a carbon, a bond to an oxygen (—O—), a double bond (=), a triple bond (≡) or (—C₂H₂—), a bond to C—C, a bond to C—N, —(CH₂)ₙ—, wherein independently each n=0, 1, 2, 3, 4, 5, 6, 7, 8, or 9; or a combination thereof.

2. The method of claim 1, wherein the method can inhibit the interaction between YTH domains and m⁶A-modified transcripts.

3. The method of claim 1, wherein a YTH domain is part of the YTH family of proteins.

4. The method of claim 1, wherein the small molecule comprises:

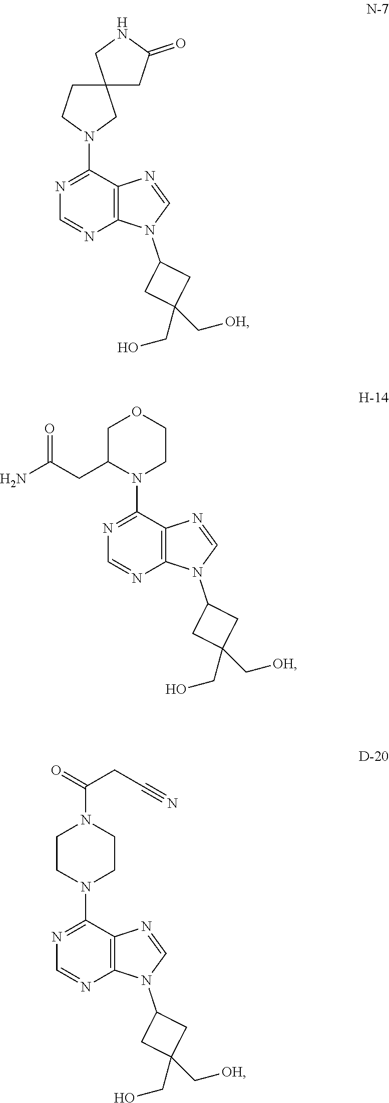 [SMILES: O=C1CC2(CCN(c3ncnc4c3ncn4C3CC(CO)(CO)C3)C2)CN1 ; NC(=O)CC1COCCN1c1ncnc2c1ncn2C1CC(CO)(CO)C1 ; N#CCC(=O)N1CCN(c2ncnc3c2ncn3C2CC(CO)(CO)C2)CC1]

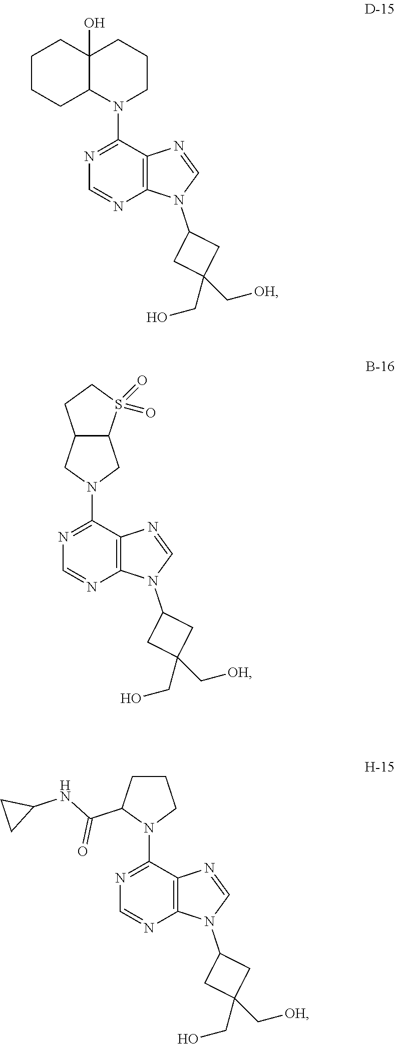 [SMILES: OCC1(CO)CC(n2cnc3c(N4CCCC5(O)CCCCC45)ncnc32)C1 ; O=S1(=O)CCC2CN(c3ncnc4c3ncn4C3CC(CO)(CO)C3)CC21 ; O=C(NC1CC1)C1CCCN1c1ncnc2c1ncn2C1CC(CO)(CO)C1]

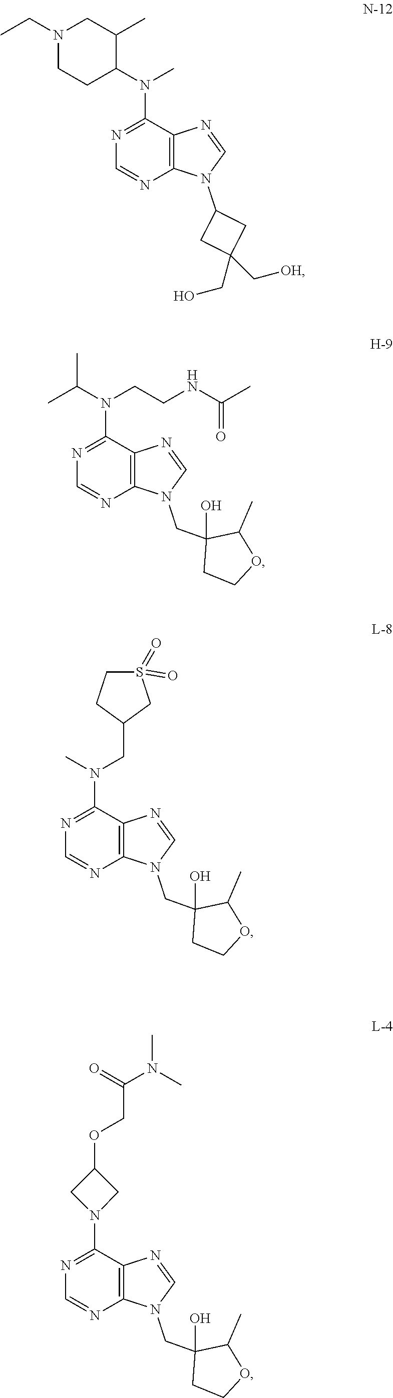 [SMILES: CCN1CCC(N(C)c2ncnc3c2ncn3C2CC(CO)(CO)C2)C(C)C1 ; CC(=O)NCCN(c1ncnc2c1ncn2CC1(O)CCOC1C)C(C)C ; CC1OCCC1(O)Cn1cnc2c(N(C)CC3CCS(=O)(=O)C3)ncnc21 ; CC1OCCC1(O)Cn1cnc2c(N3CC(OCC(=O)N(C)C)C3)ncnc21]

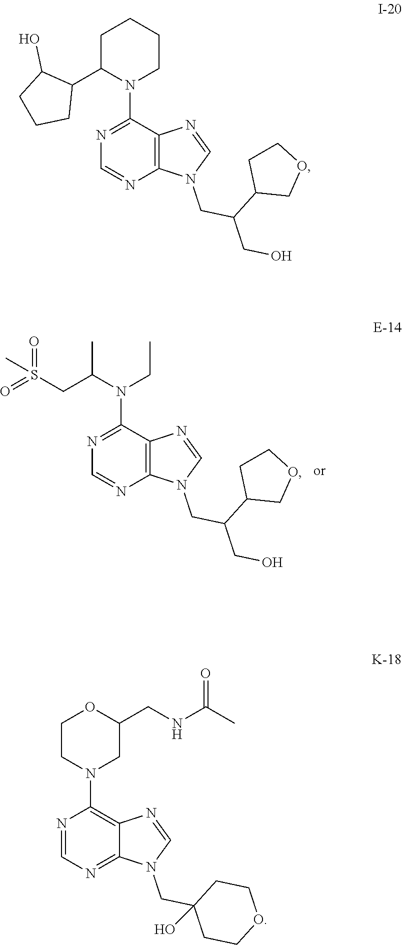 [SMILES: OCC(Cn1cnc2c(N3CCCCC3C3CCCC3O)ncnc21)C1CCOC1 ; CCN(c1ncnc2c1ncn2CC(CO)C1CCOC1)C(C)CS(C)(=O)=O ; "or" ; CC(=O)NCC1CN(c2ncnc3c2ncn3CC2(O)CCOCC2)CCO1]

5. A method for identifying pan-YTH domain inhibitors, the method comprising the steps of
(1) contacting an YTH domain of the YTHDF1 protein and FAM-labeled m6A RNA with a candidate inhibitor; and
(2) executing FP HTS (fluorescence polarization, high-throughput screening) on the candidate inhibitor; whereby a potential inhibition provided by the candidate is indicated.

6. The method of claim 5, wherein the candidate inhibitor is a small molecule compound selected from a library of diverse chemical structures.

7. The method of claim 5, wherein the FP HTS assay measures the binding affinity between the YTH domain and the candidate inhibitor by detecting changes in fluorescence polarization upon binding of a fluorescently labeled probe to the YTH domain in the presence or absence of the candidate inhibitor.

8. The method of claim 5, further comprising the step of validating the lead inhibitor in a cell-based assay to confirm inhibition of the YTH domain by measuring changes in m6A-modified mRNA levels or translation efficiency.

9. The method of claim 5, further comprising the step of determining the selectivity of the lead inhibitor by testing its ability to inhibit other YTH domain-containing proteins or unrelated RNA-binding proteins.

10. The method of claim 5, further comprising the step of optimizing the lead inhibitor by synthesizing and testing analogs with improved potency, selectivity, or pharmacokinetic properties.

11. The method of claim 5, wherein the FP HTS assay is performed in a high-throughput format using 384-well or 1536-well microplates to screen a large number of candidate inhibitors simultaneously.

12. The method of claim 5, wherein the lead inhibitor is further characterized by determining its binding mode and molecular interactions with the YTH domain using X-ray crystallography or NMR spectroscopy.

13. The method of claim 5, wherein the lead inhibitor is used as a tool compound to study the biological functions of the YTHDF1 protein and the role of m6A modification in post-transcriptional gene regulation.

14. A method of treating a disease or disorder, comprising:
contacting a pan-YTH domain in a subject with a small molecule pan-YTH domain inhibitor, wherein the small molecule pan-YTH domain inhibitor is capable of inhibiting multiple YTH domains found in a YTH family of proteins that recognize m6A-modified transcripts, thereby changing a post-transcriptional modification process.

15. The method of claim 14, wherein the disease or disorder is selected from the group consisting of cancer, viral infections, bacterial infections, fungal infections, parasitic infections, inflammatory disorders, autoimmune disorders, neurodegenerative disorders, metabolic disorders, cardiovascular disorders, respiratory disorders, and genetic disorders.

16. The method of claim 14, wherein the cancer is selected from the group consisting of leukemia, lymphoma, melanoma, lung cancer, breast cancer, colorectal cancer, prostate cancer, ovarian cancer, and brain cancer.

17. The method of claim 14, wherein the viral infection is selected from the group consisting of HIV, hepatitis B, hepatitis C, influenza, and COVID-19.

18. The method of claim 14, wherein the inflammatory disorder is selected from the group consisting of rheumatoid arthritis, inflammatory bowel disease, psoriasis, and asthma.

19. The method of claim 14, wherein the neurodegenerative disorder is selected from the group consisting of Alzheimer's disease, Parkinson's disease, Huntington's disease, and amyotrophic lateral sclerosis.

20. The method of claim 14, wherein the metabolic disorder is selected from the group consisting of obesity, diabetes, and non-alcoholic fatty liver disease.


SEQUENCE LISTING (3 sequences)

SEQ ID NO: 1 - RNA, 16 nt, synthetic construct
  source 1..16: mol_type = other RNA
  modified_base 8: mod_base = m6a
gaaccggactgtctta

SEQ ID NO: 2 - RNA, 16 nt, synthetic construct
  source 1..16: mol_type = other RNA
gaaccggactgtctta

SEQ ID NO: 3 - RNA, 16 nt, synthetic construct
  source 1..16: mol_type = other RNA
  modified_base 8: mod_base = m6a; mod_base = m6a
gaaccggactgtctta